SEQUENCE LISTING (64 sequences)

SEQ ID NO: 1 - protein, 5 aa, synthetic construct
SYYWS

SEQ ID NO: 2 - protein, 16 aa, synthetic construct
YIYYSGSTNYNPSLKS

SEQ ID NO: 3 - protein, 17 aa, synthetic construct
TTIFGVVTPNFYYGMDV

SEQ ID NO: 4 - protein, 11 aa, synthetic construct
RASQGISSYLA

SEQ ID NO: 5 - protein, 7 aa, synthetic construct
AASTLQS

SEQ ID NO: 6 - protein, 9 aa, synthetic construct
QQLNSYPLT

SEQ ID NO: 7 - protein, 125 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSS

SEQ ID NO: 8 - protein, 107 aa, synthetic construct
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIK

SEQ ID NO: 9 - protein, 455 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 10 - protein, 214 aa, synthetic construct
DIQMTQSPSFLSASVGDRVTITCRASQGISSYLAWYQQKPGKAPKFLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
RYNMN

SEQ ID NO: 12 - protein, 17 aa, synthetic construct
SISTSSNYIYYADSVKG

SEQ ID NO: 13 - protein, 7 aa, synthetic construct
GWGPFDY

SEQ ID NO: 14 - protein, 11 aa, synthetic construct
RARQSIGTAIH

SEQ ID NO: 15 - protein, 7 aa, synthetic construct
YASESIS

SEQ ID NO: 16 - protein, 9 aa, synthetic construct
QQSGSWPYT

SEQ ID NO: 17 - protein, 116 aa, synthetic construct
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 18 - protein, 107 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 19 - protein, 475 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 20 - protein, 20 aa, synthetic construct
GGSEGKSSGSGSESKSTGGS

SEQ ID NO: 21 - protein, 8 aa, synthetic construct
GGGSGGGS

SEQ ID NO: 22 - protein, 12 aa, synthetic construct
GGGSGGGSGGGS

SEQ ID NO: 23 - protein, 16 aa, synthetic construct
GGGSGGGSGGGSGGGS

SEQ ID NO: 24 - protein, 20 aa, synthetic construct
GGGSGGGSGGGSGGGSGGGS

SEQ ID NO: 25 - protein, 15 aa, synthetic construct
GGGGSGGGGSGGGGS

SEQ ID NO: 26 - protein, 20 aa, synthetic construct
GGGGSGGGGSGGGGSGGGGS

SEQ ID NO: 27 - protein, 25 aa, synthetic construct
GGGGSGGGGSGGGGSGGGGSGGGGS

SEQ ID NO: 28 - protein, 18 aa, synthetic construct
GSTSGSGKPGSGEGSTKG

SEQ ID NO: 29 - protein, 14 aa, synthetic construct
IRPRAIGGSKPRVA

SEQ ID NO: 30 - protein, 15 aa, synthetic construct
GKGGSGKGGSGKGGS

SEQ ID NO: 31 - protein, 15 aa, synthetic construct
GGKGSGGKGSGGKGS

SEQ ID NO: 32 - protein, 15 aa, synthetic construct
GGGKSGGGKSGGGKS

SEQ ID NO: 33 - protein, 15 aa, synthetic construct
GKGKSGKGKSGKGKS

SEQ ID NO: 34 - protein, 15 aa, synthetic construct
GGGKSGGKGSGKGGS

SEQ ID NO: 35 - protein, 15 aa, synthetic construct
GKPGSGKPGSGKPGS

SEQ ID NO: 36 - protein, 20 aa, synthetic construct
GKPGSGKPGSGKPGSGKPGS

SEQ ID NO: 37 - protein, 20 aa, synthetic construct
GKGKSGKGKSGKGKSGKGKS

SEQ ID NO: 38 - protein, 14 aa, synthetic construct
STAGDTHLGGEDFD

SEQ ID NO: 39 - protein, 15 aa, synthetic construct
GEGGSGEGGSGEGGS

SEQ ID NO: 40 - protein, 15 aa, synthetic construct
GGEGSGGEGSGGEGS

SEQ ID NO: 41 - protein, 15 aa, synthetic construct
GEGESGEGESGEGES

SEQ ID NO: 42 - protein, 15 aa, synthetic construct
GGGESGGEGSGEGGS

SEQ ID NO: 43 - protein, 20 aa, synthetic construct
GEGESGEGESGEGESGEGES

SEQ ID NO: 44 - protein, 18 aa, synthetic construct
GSTSGSGKPGSGEGSTKG

SEQ ID NO: 45 - protein, 19 aa, synthetic construct
PRGASKSGSASQTGSAPGS

SEQ ID NO: 46 - protein, 19 aa, synthetic construct
GTAAAGAGAAGGAAAGAAG

SEQ ID NO: 47 - protein, 19 aa, synthetic construct
GTSGSSGSGSGGSGSGGGG

SEQ ID NO: 48 - protein, 20 aa, synthetic construct
GKPGSGKPGSGKPGSGKPGS

SEQ ID NO: 49 - protein, 4 aa, synthetic construct
GSGS

SEQ ID NO: 50 - protein, 10 aa, synthetic construct
APAPAPAPAP

SEQ ID NO: 51 - protein, 20 aa, synthetic construct
APAPAPAPAPAPAPAPAPAP

SEQ ID NO: 52 - protein, 32 aa, synthetic construct
AEAAAKEAAAKEAAAAKEAAAAKEAAAAKAAA

SEQ ID NO: 53 - protein, 16 aa, synthetic construct
GTEGKSSGSGSESKST

SEQ ID NO: 54 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 55 - protein, 231 aa, synthetic construct
EPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 56 - protein, 107 aa, synthetic construct [sequence omitted]

SEQ ID NO: 57 - protein, 454 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAGTTIFGVVTPNFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 58 - protein, 474 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 59 - protein, 243 aa, synthetic construct
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 60 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 61 - protein, 104 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 62 - protein, 261 aa, synthetic construct
MWDLVLSIALSVGCTGAVPLIQSRIVGGWECEKHSQPWQVAVYSHGWAHCGGVLVHPQWVLTAAHCLKKNSQVWLGRHNLFEPEDTGQRVPVSHSFPHPLYNMSLLKHQSLRPDEDSSHDLMLLRLSEPAKITDVVKVLGLPTQEPALGTTCYASGWGSIEPEEFLRPRSLQCVSLHLLSNDMCARAYSEKVTEFMLCAGLWTGGKDTCGGDSGGPLVCNGVLQGITSWGPEPCALPEKPAVYTKVVHYRKWIKDTIAANP

SEQ ID NO: 63 - protein, 120 aa, synthetic construct
QVQLVESGGGVVQPGRSLRLSCAASGFTFSSYNMNWVRQAPGKGLEWVAIIYYDESNKYYADSVKGRFTISRDISKNTLYLQMNSLRAEDTAVYYCARERGRDYYGMDVWGQGTTVTVSS

SEQ ID NO: 64 - protein, 107 aa, synthetic construct
SYELMQPPSVSVSPGQTARITCSGDALPKQYAYWYQQKPGQAPVLVIYKDSERPSGIPVRFSGSSSGTTVTLTITGVQAEDEADYYCQSADSSGTYVFGTGTKVTVL

(19) United States
(12) Patent Application Publication
(10) Pub. No.: US 2026/0062500 A1
(43) Pub. Date: Mar. 5, 2026

(54) BISPECIFIC ANTIBODY TARGETING HK2 AND CD3 FOR THE TREATMENT OF PROSTATE CANCER

(71) Applicant: Janssen Biotech, Inc., Horsham, PA (US)

(72) Inventors: Sherry Chia-E WANG, San Francisco, CA (US); Victor Manuel VILLALOBOS, New Hope, PA (US); Pharavee JAIPRASART, North Wales, PA (US)

(21) Appl. No.: 19/297,431

(22) Filed: Aug. 12, 2025

Related U.S. Application Data
(60) Provisional application No. 63/682,768, filed on Aug. 13, 2024.
(60) Provisional application No. 63/720,852, filed on Nov. 15, 2024.
(60) Provisional application No. 63/735,486, filed on Dec. 18, 2024.
(60) Provisional application No. 63/746,592, filed on Jan. 17, 2025.
(60) Provisional application No. 63/790,940, filed on Apr. 18, 2025.
(60) Provisional application No. 63/805,015, filed on May 13, 2025.

(51) Int. Cl. C07K 16/40; A61K 39/00; A61K 45/06; A61P 35/04; C07K 16/28

(52) U.S. Cl. CPC C07K 16/40; A61K 45/06; A61P 35/04; C07K 16/2809; A61K 2039/505; A61K 2039/54; A61K 2039/545

(57) ABSTRACT

The present invention relates to methods of treating prostate cancer in a subject in need thereof, comprising administering a therapeutically effective amount of a KLK2xCD3 bispecific antibody or bispecific binding fragment thereof to the subject to treat the prostate cancer.

### CROSS-REFERENCE TO RELATED APPLICATIONS

This application claims priority to U.S. Provisional Application No. 63/682,768 filed on Aug. 13, 2024, U.S. Provisional Application No. 63/720,852 filed on Nov. 15, 2024, U.S. Provisional Application No. 63/735,486 filed on Dec. 18, 2024, U.S. Provisional Application No. 63/746,592 filed on Jan. 17, 2025, U.S. Provisional Application No. 63/790,940 filed on Apr. 18, 2025, and U.S. Provisional Application No. 63/805,015 filed on May 13, 2025, the disclosures of each of which are hereby incorporated by reference in their entireties.

### SEQUENCE LISTING

The instant application contains a Sequence Listing which has been submitted electronically in XML format and is hereby incorporated by reference in its entirety. The XML copy, created on Aug. 4, 2025, is named JBI6921USNP1-SL.xml and is 60,783 bytes in size.

### TECHNICAL FIELD

The disclosure provided herein relates to methods of treating prostate cancer using bispecific antibodies that bind to kallikrein related peptidase 2 (hK2) and cluster determinant 3 (CD3).

### BACKGROUND

Prostate cancer (PC) is the second most frequently diagnosed cancer and the sixth leading cause of cancer death in males, accounting for 14% (903,500) of the total new cancer cases and 6% (258,400) of the total cancer deaths in males worldwide. Historically, androgen depletion therapy (ADT) has been the standard of care for patients with metastatic prostate cancer. However, in the advanced form of prostate cancer, it becomes metastatic and spreads beyond the prostate to other parts of the body. With metastatic Castration-Resistant Prostate Cancer (mCRPC), hormone therapy no longer stops cancer growth. End stage PC (castration-resistant prostate cancer) currently has no curative treatment option.

Antibody-based therapeutics have been used successfully to treat a variety of diseases, including cancer. One particular approach is to engineer a bispecific antibody with a first antigen binding domain that engages CD3 and a second antigen binding domain that engages an antigen associated with or upregulated on cancer cells so that the bispecific antibody redirects CD3⁺ T cells to destroy the cancer cells. Bispecific antibodies that bind specifically to kallikrein related peptidase 2 (KLK2, hK2) and CD3 (KLK2xCD3 bispecific antibodies) and uses thereof for treating cancer have been described. See, e.g., WO2021019389.

It is important for cancer therapy to enhance anti-tumor activity by specifically destroying tumor cells while minimizing peripheral toxicity. In this context, it is crucial that only T cells in the presence of the target tumor cells are stimulated to mediate killing of the target tumor cells. Nondiscriminatory stimulation of T cells may lead to off-tumor, on-target toxicity. For instance, inappropriate activation (e.g. overactivation) of T cells by bispecific antibodies that engage the T cell (such as bispecific CD3 antibodies) can lead to unwanted adverse events. Cytokine Release Syndrome (CRS) is an adverse event that arises from overactivation of T cells, which in turn leads to excessive cytokine release that can be particularly serious and lead to multi-organ failure and death if it is managed properly (Shah et al. Front. Immunol. (2023) 14: 1-23). This phenomenon of adverse events, and in particular CRS, being associated with bispecific CD3 antibodies has been observed in several different contexts For example, a recent report of a clinical trial in which a STEAP1 x CD3 antibody was administered to prostate cancer patients by intravenous infusion found that CRS was the most common adverse event in these patients (Kelly et al. Cancer Discov (2024) 14 (1): 76-89).

There remains a need, therefore, for novel therapeutic options, such as new dosage and treatment regimens that have an optimal benefit-risk profile in the treatment of prostate cancer.

### SUMMARY OF INVENTION

The invention provides a method of treating prostate cancer in a human subject in need thereof, wherein the method comprises administering a therapeutically effective amount of an isolated anti-hK2/anti-CD3 bispecific antibody or a bispecific fragment thereof to the subject at a treatment dose by intravenous (IV) infusion, wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

The invention also provides an isolated anti-hK2/anti-CD3 bispecific antibody or a bispecific fragment thereof wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18,
   for use in a method of treating prostate cancer in a human subject in need thereof, wherein the method comprises administering a therapeutically effective amount of an isolated anti-hK2/anti-CD3 bispecific antibody or a bispecific fragment thereof to the subject at a treatment dose by intravenous (IV) infusion.

Unlike previous T-cell engagers for PC, the anti-hK2/anti-CD3 bispecific antibody of the application administered to prostate cancer patients humans by intravenous (IV) infusion (e.g., pasritamig administered by IV infusion to prostate cancer patients in a first-in-human trial) demonstrated a favorable safety profile with low rates of treatment-related adverse events and very low rates of [low-grade]cytokine release syndrome (CRS), enabling outpatient dosing on a convenient schedule. The anti-hK2/anti-CD3 bispecific antibody of the application (e.g., pasritamig in a first-in-human trial) achieved durable responses and promising radiographic progression-free survival in heavily pretreated participants, warranting further development in later stage studies. In addition to overall low CRS rates, the administration by IV infusion of the anti-hK2/anti-CD3 bispecific antibody of the application (e.g., of pasritamig) resulted in lower rates of CRS and fatigue than subcutaneous (SC) dosing and eliminated injection-site reactions.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a simulated mean PK profile for KLK2xCD3 bispecific antibody serum concentrations in humans following a single SC Dose of 6.5 μg/kg. Abbreviations: MABEL=minimum anticipated biologic effect level; PK=pharmacokinetic; SC=subcutaneous.

FIG. 2 illustrates the mean (SD) serum concentrations of the KLK2xCD3 bispecific antibody after Administration of the First Treatment Dose of KLK2xCD3 bispecific antibody. Key: IV=intravenous; SC=subcutaneous; SD=standard deviation; Q3W=once every 3 weeks; Q6W=once every 6 weeks. Mean±SD are presented if evaluable participants ≥3.

FIG. 3 is a graphical representation of the number of patients displaying treatment emergent adverse events (TEAEs). A comparison between the cohorts that were administered the KLK2xCD3 bispecific antibody intravenously compared to those administered the KLK2xCD3 bispecific antibody subcutaneously. AETOXGR=Adverse Event Toxicity Grade according to National Cancer Institute Common Terminology Criteria for Adverse Events version 5.0.

FIG. 4 is a graphical comparison of the patient incidence rate for TEAEs and TRAEs in cohorts which were administered the KLK2xCD3 bispecific antibody intravenously. AETOXGR=Adverse Event Toxicity Grade according to National Cancer Institute Common Terminology Criteria for Adverse Events version 5.0.

### DETAILED DESCRIPTION

The disclosed methods can be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to +10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

The transitional terms “comprising.” “consisting essentially of,” and “consisting of” are intended to connote their generally accepted meanings in the patent vemacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

It should also be understood that the terms “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the term “adverse event” (AE) refers to any untoward medical occurrence in a patient administered a pharmaceutical product and which does not necessarily have a causal relationship with the treatment. According to some embodiments of the invention, AEs are rated on a 5-point scale of increasing severity using the following definitions according to the National Cancer Institute Common Terminology Criteria for Adverse Events (NCI-CTCAE) version 5.0:
Grade 1 (mild): referring to an AE that is asymptomatic or has mild symptoms which are easily tolerated by the subject, which cause minimal discomfort and does not interfere with everyday activities.
Grade 2 (moderate): referring to an AE that is sufficiently discomforting to interfere with instrumental activities of daily living (such as preparing meals, shopping for groceries or clothes, using the telephone, managing money etc.) and intervention may be needed.
Grade 3 (severe): referring to an AE that is severe but not immediately life-threatening but is limiting to self-care activities of daily living (such as bathing, dressing, feeding self, using the toilet, taking medications etc.). Hospitalization of prolongation of hospitalization can occur.
Grade 4 (life threatening) referring to an AE that has life-threatening consequences and which requires urgent intervention
Grade 5: referring to death related to an AE.

Examples of AEs include Injection Site Reaction (ISR), fatigue, nausea, anemia, constipation, back pain, arthralgia and pyrexia (or fever). These AEs may be graded according to the National Cancer Institute Common Terminology Criteria for Adverse Events (NCI-CTCAE) version 5.0, published 27 Nov. 2017 (which is available, for example at https://ctep.cancer.gov/protocoldevelopment/electronic_applications/docs/ctcae_v5_quick_reference_5x7.pdf) and incorporated by reference herein. NCI-CTCAE is a standard classification and severity grading scale for adverse events in clinical trials and oncology settings.

Other examples of AE are Cytokine Release Syndrome (CRS) and immune effector cell-associated neurotoxicity syndrome (ICANS). These AEs may be graded according to the American Society for Transplantation and Cellular Therapy (ASTCT) guidelines, which is incorporated by reference (Lee et al. ASTCT Consensus Grading for Cytokine Release Syndrome and Neurologic Toxicity Associated with Immune Effector Cells. Biol Blood Marrow Transplant, 2019 April; 25(4):625-638).

“Antigen binding fragment” or “antigen binding domain” refers to a portion of the protein that binds an antigen. Antigen binding fragments may be synthetic, enzymatically obtainable or genetically engineered polypeptides and include portions of an immunoglobulin that bind an antigen, such as the VH, the VL, the VH and the VL, Fab, Fab′, F(ab′)2, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) may be linked together via a synthetic linker to form various types of single antibody designs where the VH/VL domains may pair intramolecularly, or intermolecularly in those cases when the VH and VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgAQ1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains. In certain embodiments, the antibodies of the disclosure are isolated from at least one component of a sample (e.g., a cell culture) from which the antibody is obtained.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Bispecific anti-hK2/anti-CD3 antibody”, “hk2/CD3 antibody”, “hk2xCD3 antibody,” “anti-hK2/anti-CD3 protein,” “KLK2xCD3 bispecific antibody” and the like refer to an antibody that binds hk2 and CD3 and that comprises at least one binding domain specifically binding hK2 and at least one binding domain specifically binding CD3. The domains specifically binding hK2 and CD3 are typically VH/VL pairs. The bispecific anti-hk2xCD3 antibody may be monovalent in terms of its binding to either hk2 or CD3.

As used herein, an “isolated anti-hK2/anti-CD3 bispecific antibody or a bispecific fragment thereof” refers to an anti-hK2/anti-CD3 bispecific antibody or a bispecific fragment thereof that is isolated from at least one component of a sample (e.g., a cell culture) from which the bispecific antibody or a bispecific fragment thereof is obtained.

“Cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al. (1970) J Exp Med 132: 211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton J Bmol Biol 263: 800-15, 1996). The correspondence between the various delineations and variable region numbering is described (see e.g., Lefranc et al. (2003) Dev Comp Immunol 27: 55-77; Honegger and Pluckthun, J Mol Biol (2001) 309:657-70; International ImMunoGeneTics (IMGT) database). Available programs such as abYsis by UCL Business PLC may be used to delineate CDRs. The term “CDR”, “HCDR1”, “HCDR2”, “HCDR3”, “LCDR”, “LCDR2” and “LCDR3” as used herein includes CDRs defined by any of the methods described supra, Kabat, Chothia, IMGT or AbM, unless otherwise explicitly stated in the specification.

“CD3” refers to an antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. All references to proteins, polypeptides and protein fragments herein are intended to refer to the human version of the respective protein, polypeptide or protein fragment unless explicitly specified as being from a non-human species. Thus, “CD3” means human CD3 unless specified as being from a non-human species, e.g., “mouse CD3” “monkey CD3,” etc.

Throughout the specification, “CD3-specific” or “specifically binds CD3” or “anti-CD3 antibody” refers to antibodies that bind specifically to a CD3-epsilon polypeptide, including antibodies that bind specifically to the CD3-epsilon extracellular domain (ECD). CD3-epsilon, together with CD3-gamma, -delta and -zeta, and the T-cell receptor alpha/beta and gamma/delta heterodimers, forms the T-cell receptor-CD3 complex. This complex plays an important role in coupling antigen recognition to several intracellular signal-transduction pathways. The CD3 complex mediates signal transduction, resulting in T cell activation and proliferation. CD3 is required for the immune response. The amino acid sequence of a human CD3 epsilon is shown in SEQ ID NO: 60. The amino acid sequence of a human CD3 epsilon extracellular domain is shown in SEQ ID NO: 61.

“Decrease,” “lower,” “lessen,” “reduce,” or “abate” refers generally to the ability of a test molecule to mediate a reduced response (i.e., downstream effect) when compared to the response mediated by a control or a vehicle. Exemplary responses are T cell expansion. T cell activation or T-cell mediated tumor cell killing or binding of a protein to its antigen or receptor, enhanced binding to a Fcγ or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7, 1.8, etc.).

“Dose limiting toxicity” or “DLT” refers to side effects of a treatment that are serious enough to prevent an increase in dose or level of that treatment. The DLT evaluation period is typically defined as the first 21 days of receiving a treatment dose. Examples of DLTs are as follows:
Neutrophil count decreased (this includes either febrile neutropenia or Neutropenia of Grade 4 for 7 days or more);
Platelet count decreases leading to Grade 3 or higher thrombocytopenia with bleeding or any Grade 4 thrombocytopenia with duration 7 days or more;
Any non-hematological toxicity of Grade ≥3, with the following exceptions:
Grade 3 fatigue, asthenia, fever, or constipation, lasting less than 7 days with best supportive care;
Grade 3 nausea/vomiting or diarrhea lasting less than 72 hours with adequate antiemetic and other supportive care;
Grade 3 or higher hypertension that can be controlled by medical management in 7 days or less;
Grade 3 or higher alanine aminotransferase (ALT) or aspartate aminotransferase (AST) that resolves to Grade 1 or baseline within 7 days, unless criteria for Hy's law are met b, d;
Isolated Grade 3 or higher alkaline phosphatase or gamma-glutamyl transferase (GGT) increase that returns to Grade 1 or baseline within 7 days;
Grade 3 or higher lipase or amylase increase not associated with clinical or radiological evidence of pancreatitis;
Grade 3 or higher electrolyte abnormalities that last up to 72 hours, are not clinically complicated, and either resolve spontaneously or respond to conventional medical interventions; and/or
First occurrence of Grade 3 systemic administration-related reactions (sARR) or cytokine release syndrome (CRS).

“Enhance,” “promote,” “increase,” “expand” or “improve” refers generally to the ability of a test molecule to mediate a greater response (i.e., downstream effect) when compared to the response mediated by a control or a vehicle. Exemplary responses are T cell expansion, T cell activation or T-cell mediated tumor cell killing or binding of a protein to its antigen or receptor, enhanced binding to a Fcγ or enhanced Fc effector functions such as enhanced ADCC, CDC and/or ADCP. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more (including all integers and decimal points in between and above 1, e.g., 1.5, 1.6, 1.7, 1.8, etc.). [0039]“dAb” or “dAb fragment” refers to an antibody fragment composed of a VH domain (Ward et al., Nature 341:544 546 (1989)).

“Fab” or “Fab fragment” refers to an antibody fragment composed of VH, CH1, VL and CL domains.

“F(ab′)2” or “F(ab′)2 fragment” refers to an antibody fragment containing two Fab fragments connected by a disulfide bridge in the hinge region.

“Fd” or “Fd fragment” refers to an antibody fragment composed of VH and CH1 domains.

“Fv” or “Fv fragment” refers to an antibody fragment composed of the VH and the VL domains from a single arm of the antibody. Fv fragments lack the constant regions of Fab (CH1 and CL) regions. The VH and VL in Fv fragments are held together by non-covalent interactions.

“Fc” polypeptide” of a dimeric Fc refers to one of the two polypeptide forming the dimeric Fc domain. For example, an Fc polypeptide of a dimeric IgG FC comprises an IgG CH2 and an IgG CH3 constant domain sequence).

“Flat dose” refers to a dose that is administered to a subject without correction for the subject's specific body weight or body surface area. A flat dose, sometimes referred to as a fixed dose, is therefore provided as an absolute amount of the agent (e.g., mg drug), and not as a weight-based amount that accounts for the subject's specific weight (e.g. μg/kg or μg drug per kg body weight). For example, a subject weighing 65 kg may be administered the same flat dose in milligrams as a subject weighing 85 kg. A flat dose may be administered according to a pre-defined class or category of body weight, but is not modified according to the subject's specific weight. For example, a “Flat Dose A” may be administered if a patient is greater than a pre-defined threshold weight, whereas a different “Flat Dose B” may be administered if the patent is less than the pre-defined threshold weight.

“Full length antibody” is comprised of two heavy chains (HC) and two light chains (LC) inter-connected by disulfide bonds as well as multimers thereof (e.g. IgM). Each heavy chain is comprised of a heavy chain variable domain (VH) and a heavy chain constant domain, the heavy chain constant domain comprised of subdomains CH1, hinge, CH2 and CH3. Each light chain is comprised of a light chain variable domain (VL) and a light chain constant domain (CL). The VH and the VL may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Homodimerization” as used herein in the context of antibodies refers to an interaction of two heavy chains having identical CH3 amino acid sequences. “Homodimer” as used herein refers to an antibody having two heavy chains with identical CH3 amino acid sequences.

“Heterodimerization” as used herein in the context of antibodies refers to an interaction of two heavy chains having non-identical CH3 amino acid sequences. “Heterodimer” as used herein refers to an antibody having two heavy chains with non-identical CH3 amino acid sequences.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“In combination with” means that two or more therapeutic agents are be administered to a subject together in a mixture, concurrently as single agents or sequentially as single agents in any order.

“Intravenous infusion” refers to the administration of a treatment directly into a subject's bloodstream through a vein, for example using a needle or catheter. An intravenous infusion can be delivered over a set period of time, ranging from minutes to several hours.

“Isolated” refers to a homogenous population of molecules (such as synthetic polynucleotides or polypeptides) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Kallikrein related peptidase 2” or “KLK2” or “hK2” refers to a human protein which is also called kallikrein-2, grandular kallikrein 2, or HK2. hK2 is produced as a preproprotein and cleaved during proteolysis to generate active protease. All hK2 isoforms and variants are encompassed in “hK2”. The amino acid sequences of various isoforms are retrievable from public sources, such as GenBank accession numbers NP_005542.1, NP_001002231.1 and NP_001243009. The amino acid sequence of a full length hK2 is shown in SEQ ID NO: 62 The sequence includes the signal peptide (residues 1-18) and the pro-peptide region (residues 19-24).

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Multispecific” refers to a molecule, such as an antibody that specifically binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

“Pharmaceutical combination” refers to a combination of two or more active ingredients administered either together or separately.

“Pharmaceutical composition” refers to a composition that results from combining an active ingredient and a pharmaceutically acceptable carrier.

“Pharmaceutically acceptable carrier” or “excipient” refers to an ingredient in a pharmaceutical composition, other than the active ingredient, which is nontoxic to a subject. Exemplary pharmaceutically acceptable carriers are a buffer, stabilizer or preservative.

“Prevent,” “preventing,” “prevention,” or “prophylaxis” of a disease or disorder means preventing that a disorder occurs in a subject.

“Protein” or “polypeptide” are used interchangeably herein are refers to a molecule that comprises one or more polypeptides each comprised of at least two amino acid residues linked by a peptide bond. Protein may be a monomer, or may be protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation. Protein may be recombinantly expressed.

“Prostate specific antigen” or “PSA” refers to a glycoprotein found in the epithelial cells of the prostate gland. It is usually found in small quantities in the blood of healthy patients. Elevated levels of PSA in the blood can indicate that a patient may have a prostate disorder, such as prostate cancer.

“Recombinant” refers to polynucleotides, polypeptides, vectors, viruses and other macromolecules that are prepared, expressed, created or isolated by recombinant means.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

“Single chain Fv” or “scFv” refers to a fusion protein comprising at least one antibody fragment comprising a light chain variable region (VL) and at least one antibody fragment comprising a heavy chain variable region (VH), wherein the VL and the VH are contiguously linked via a polypeptide linker, and capable of being expressed as a single chain polypeptide. Unless specified, as used herein, a scFv may have the VL and VH variable regions in either order, e.g., with respect to the N-terminal and C-terminal ends of the polypeptide, the scFv may comprise VL-linker-VH or may comprise VH-linker-VL. [0067]“(scFv)2” or “tandem scFv” or “bis-scFv” fragments refers to a fusion protein comprising two light chain variable region (VL) and two heavy chain variable region (VH), wherein the two VL and the two VH regions are contiguously linked via polypeptide linkers, and capable of being expressed as a single chain polypeptide. The two VL and two VH regions fused by peptide linkers form a bivalent molecule VLA-linker-VHA-linker-VLB-linker-VHB to form two binding sites, capable of binding two different antigens or epitopes concurrently.

“Specifically binds,” “specific binding,” “specifically binding” or “binds” refer to a proteinaceous molecule binding to an antigen or an epitope within the antigen with greater affinity than for other antigens. Typically, the proteinaceous molecule binds to the antigen or the epitope within the antigen with an equilibrium dissociation constant (KD) of about 1×10⁻⁷ M or less, for example about 5×10⁻⁸M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the KD that is at least one hundred fold less than its KD for binding to a non-specific antigen (e.g., BSA, casein). In the context of the prostate neoantigens described here, “specific binding” refers to binding of the proteinacous molecule to the prostate neoantigen without detectable binding to a wild-type protein the neoantigen is a variant of.

“Step-up dose” refers to a dose of an active agent that is administered to a subject prior to a treatment dose. A step-up dose is lower than the treatment dose. A dosing regimen comprising one or more step-up doses followed by higher treatment doses may be considered a “priming” dose strategy.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“Therapeutically effective amount” or “effective amount” used interchangeably herein, refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual. Example indicators of an effective therapeutic or combination of therapeutics that include, for example, improved wellbeing of the patient, reduction of a tumor burden, arrested or slowed growth of a tumor, reduction in PSA levels, and/or absence of metastasis of cancer cells to other locations in the body.

“Treat,” “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Treatment dose” refers to a dose of the active agent that is administered to a subject to treat a disease. A treatment dose may be administered at a regular dosing interval on a repetitive basis (e.g. weekly (i.e. once a week), once every 2 weeks, once every 3 weeks, once every 4 weeks, once every 5 weeks, once every 6 weeks etc.). A treatment dose may be preceded by one or more step-up doses.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation may arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

“Variant,” “mutant” or “altered” refers to a polypeptide or a polynucleotide that differs from a reference polypeptide or a reference polynucleotide by one or more modifications, for example one or more substitutions, insertions or deletions.

“Weight-based dose” refers to when a dose, such as a treatment dose or a step-up dose, is determined using the body weight of the subject. Such dosages can, for example, be based on the mg/kg dosages provided above according to the following: dose (mg/kg)×body weight (e.g., 50-100 kg).

“Q6W” is understood in accordance with its ordinary meaning in the field and refers to a dose frequency of once every 6 weeks, or of once every 42 days+/−3 days, more specifically once every 42 days.

Unless indicated otherwise, the numbering of amino acid residues in the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated.

Administration of hK2xCD3 Bispecific Antibody

The inventors have developed new regimens for administering an isolated anti-hK2/anti-CD3 bispecific antibody or a bispecific fragment thereof to treat prostate cancer in a subject.

In particular, the inventors have found that administration of an anti-hK2/anti-CD3 bispecific antibody by intravenous (IV) infusion may be associated with fewer side-effects, such as adverse events that are described in the examples when compared to patients receiving the same anti-hk2/anti-CD3 bispecific antibody by subcutaneous (SC) administration. For instance, side effects such as cytokine release syndrome (CRS) that can be associated with T-cell redirecting therapies were observed far less frequently in patients receiving intravenous administration of the antibody. Reductions in other adverse events such as injection site reaction (ISR), nausea, anemia and fatigue were also observed in patients receiving IV infusion. A reduction in adverse events is advantageous for several reasons. The patient is less likely to experience complications that, if severe, can lead to treatment being stopped. A reduced likelihood of adverse events can also mean that patients may be treated as outpatients, thereby reducing the pressure on inpatient services in healthcare settings.

Accordingly, the invention provides a method of treating prostate cancer in a human subject in need thereof, wherein the method comprises administering a therapeutically effective amount of an isolated anti-hK2/anti-CD3 bispecific antibody or a bispecific fragment thereof to the subject at a treatment dose by intravenous (IV) infusion, wherein the isolated anti-hK2/anti-CD3 bispecific antibody or bispecific fragment thereof comprises a first antigen binding domain that binds specifically to hK2, and a second antigen binding domain that binds specifically to CD3, as described elsewhere herein.

The invention also provides an isolated anti-hK2/anti-CD3 bispecific antibody or a bispecific fragment thereof comprising a first antigen binding domain that binds specifically to hK2, and a second antigen binding domain that binds specifically to CD3, as disclosed elsewhere herein, for use in any of the methods disclosed herein. For instance, the invention provides an isolated anti-hK2/anti-CD3 bispecific antibody or a bispecific fragment thereof comprising a first antigen binding domain that binds specifically to hK2, and a second antigen binding domain that binds specifically to CD3, for use in a method treating prostate cancer in a human subject in need thereof, wherein the method comprises administering a therapeutically effective amount of the isolated anti-hK2/anti-CD3 bispecific antibody or a bispecific fragment thereof to the subject at a treatment dose by intravenous (IV) infusion.

An intravenous infusion of the isolated anti-hK2/anti-CD3 bispecific antibody or a bispecific fragment thereof may be administered over several minutes to several hours. The inventors have found that administration of an initial dose of an isolated anti-hK2/anti-CD3 bispecific antibody over a period of one hour followed by administration of subsequent infusions over a period of 30 minutes are suitable timings for administration. Accordingly, in some embodiments, the treatment dose is administered by intravenous (IV) infusion over a period of at least one hour. In certain such embodiments, the treatment dose is administered by intravenous (IV) infusion over a period of about one hour.

In some embodiments, the treatment dose is administered by intravenous (IV) infusion over a period of at least 30 minutes. In certain such embodiments, the treatment dose is administered by intravenous (IV) infusion over a period of about 30 minutes.

In some embodiments, the first treatment dose is administered by intravenous infusion over a period of at least one hour (for example over a period of about one hour) and any subsequent treatment dose is administered by intravenous infusion over a period of at least 30 minutes (for example over a period of about 30 minutes).

### Dosing

The isolated anti-hK2/anti-CD3 bispecific antibody or a bispecific fragment thereof is administered at a treatment dose to treat prostate cancer in a subject. The inventors have found that certain treatment doses may be suitable for treatment of prostate cancer.

Accordingly, in some embodiments, the treatment dose is at least about 75 mg per administrations.

In some embodiments, the treatment dose is between about 75 mg and about 900 mg per administration, for example about 75 mg, about 100 mg, about 150 mg, about 200 mg, about 250 mg, about 300 mg, about 400 mg, about 500 mg, about 600 mg, about 700 mg, about 800 mg, or about 900 mg per administration.

In some embodiments, the treatment dose is between about 75 mg and about 600 mg per administration, for example about 75 mg, about 100 mg, about 150 mg, about 200 mg, about 250 mg, about 300 mg, about 400 mg, about 500 mg, or about 600 mg per administration.

In some embodiments, the treatment dose is between about 75 mg and about 300 mg per administration, for example about 75 mg, about 100 mg, about 150 mg, about 200 mg, about 250 mg, or about 300 mg per administration.

In some embodiments, the treatment dose is between about 75 mg and about 250 mg per administration, for example about 75 mg, about 100 mg, about 150 mg, about 200 mg, or about 250 mg per administration.

In some embodiments, the treatment dose is between about 75 mg and about 200 mg per administration, for example about 75 mg, about 100 mg, about 150 mg, or about 200 mg, per administration.

In some embodiments, the treatment dose is between about 75 mg and about 150 mg per administration, for example about 75 mg, about 100 mg, or about 150 mg per administration.

In a particular embodiment, the treatment dose is about 75 mg per administration.

In some embodiments, the treatment dose is at least about 150 mg per administration.

In some embodiments, the treatment dose is between about 150 mg to about 900 mg, for example about 150 mg, about 200 mg, about 250 mg, about 300 mg, about 400 mg, about 500 mg, about 600 mg, about 700 mg, about 800 mg or about 900 mg per administration.

In some embodiments, the treatment dose is between about 150 mg to about 600 mg, for example about 150 mg, about 200 mg, about 250 mg, about 300 mg, about 400 mg, about 500 mg, or about 600 mg per administration.

In some embodiments, the treatment dose is between about 150 mg to about 300 mg, for example about 150 mg, about 200 mg, about 250 mg, or about 300 mg per administration.

In some embodiments, the treatment dose is between about 150 mg to about 250 mg, for example about 150 mg, about 200 mg, or about 250 mg per administration.

In some embodiments, the treatment dose is about 150 mg to about 200 mg, for example about 150 mg, or about 200 mg per administration.

In a particular embodiment, the treatment dose is about 150 mg per administration.

In some embodiments, the treatment dose is at least about 200 mg per administration.

In some embodiments, the treatment dose is between about 200 mg to about 900 mg, for example about 200 mg, about 250 mg, about 300 mg, about 400 mg, about 500 mg, about 600 mg, about 700 mg, about 800 mg or about 900 mg per administration.

In some embodiments, the treatment dose is between about 200 mg to about 600 mg, for example about 200 mg, about 250 mg, about 300 mg, about 400 mg, about 500 mg, or about 600 mg per administration.

In some embodiments, the treatment dose is between about 200 mg to about 300 mg, for example about 200 mg, about 250 mg, or about 300 mg per administration.

In some embodiments, the treatment dose is between about 200 mg to about 250 mg, for example about 200 mg, or about 250 mg per administration.

In a particular embodiment, the treatment dose is about 200 mg per administration.

In some embodiments, the treatment dose is at least about 250 mg per administration.

In some embodiments, the treatment dose is between about 250 mg to about 900 mg, for example about 250 mg, about 300 mg, about 400 mg, about 500 mg, about 600 mg, about 700 mg, about 800 mg or about 900 mg per administration.

In some embodiments, the treatment dose is between about 250 mg to about 600 mg, for example about 250 mg, about 300 mg, about 400 mg, about 500 mg, or about 600 mg per administration.

In some embodiments, the treatment dose is between about 250 mg to about 300 mg, for example about 250 mg, or about 300 mg per administration.

In a particular embodiment, the treatment dose is about 250 mg per administration.

In some embodiments, the treatment dose is at least about 300 mg per administration.

In some embodiments, the treatment dose is between about 300 to about 900 mg per administration, for example about 300 mg, about 400 mg, about 500 mg, about 600 mg, about 700 mg, about 800 mg or about 900 mg per administration.

In some embodiments, the treatment dose is between about 300 mg to about 600 mg, for example about 300 mg, about 400 mg, about 500 mg, or about 600 mg per administration.

In a particular embodiment, the treatment dose is about 300 mg per administration.

In some embodiments, the treatment dose is at least about 600 mg per administration.

In some embodiments, the treatment dose is between about 600 to about 900 mg per administration, for example about 600 mg, about 700 mg, about 800 mg or about 900 mg per administration.

In a particular embodiment, the treatment dose is about 600 mg per administration.

In a particular embodiment, the treatment dose is about 900 mg per administration.

The treatment dose of the isolated anti-hK2/anti-CD3 bispecific antibody or bispecific fragment thereof may be a flat dose, as defined herein. Flat dosing may provide advantages compared to dosing according to body weight, such as reduced preparation time, and simpler administration and manufacturing.

In some embodiments, the treatment dose is a flat dose of at least about 75 mg per administrations.

In some embodiments, the treatment dose is a flat dose of between about 75 mg and about 900 mg per administration, for example a flat dose of about 75 mg, a flat dose of about 100 mg, a flat dose of about 150 mg, a flat dose of about 200 mg, a flat dose of about 250 mg, a flat dose of about 300 mg, a flat dose of about 400 mg, a flat dose of about 500 mg, a flat dose of about 600 mg, a flat dose of about 700 mg, a flat dose of about 800 mg, or a flat dose of about 900 mg per administration.

In some embodiments, the treatment dose is a flat dose of between about 75 mg and about 600 mg per administration, for example a flat dose of about 75 mg, a flat dose of about 100 mg, a flat dose of about 150 mg, a flat dose of about 200 mg, a flat dose of about 250 mg, a flat dose of about 300 mg, a flat dose of about 400 mg, a flat dose of about 500 mg, or a flat dose of about 600 mg per administration.

In some embodiments, the treatment dose is a flat dose of between about 75 mg and about 300 mg per administration, for example a flat dose of about 75 mg, a flat dose of about 100 mg, a flat dose of about 150 mg, a flat dose of about 200 mg, a flat dose of about 250 mg, or a flat dose of about 300 mg per administration.

In some embodiments, the treatment dose is a flat dose of between about 75 mg and about 250 mg per administration, for example a flat dose of about 75 mg, a flat dose of about 100 mg, a flat dose of about 150 mg, a flat dose of about 200 mg, or a flat dose of about 250 mg per administration.

In some embodiments, the treatment dose is a flat dose of between about 75 mg and about 200 mg per administration, for example a flat dose of about 75 mg, a flat dose of about 100 mg, a flat dose of about 150 mg, or a flat dose of about 200 mg, per administration.

In some embodiments, the treatment dose is a flat dose of between about 75 mg and about 150 mg per administration, for example a flat dose of about 75 mg, a flat dose of about 100 mg, or a flat dose of about 150 mg per administration.

In a particular embodiment, the treatment dose is a flat dose of about 75 mg per administration.

In some embodiments, the treatment dose is a flat dose of at least about 150 mg per administration.

In some embodiments, the treatment dose is a flat dose of between about 150 mg to about 900 mg, for example a flat dose of about 150 mg, a flat dose of about 200 mg, a flat dose of about 250 mg, a flat dose of about 300 mg, a flat dose of about 400 mg, a flat dose of about 500 mg, a flat dose of about 600 mg, a flat dose of about 700 mg, a flat dose of about 800 mg or a flat dose of about 900 mg per administration.

In some embodiments, the treatment dose is a flat dose of between about 150 mg to about 600 mg, for example a flat dose of about 150 mg, a flat dose of about 200 mg, a flat dose of about 250 mg, a flat dose of about 300 mg, a flat dose of about 400 mg, a flat dose of about 500 mg, or a flat dose of about 600 mg per administration.

In some embodiments, the treatment dose is a flat dose of between about 150 mg to about 300 mg, for example a flat dose of about 150 mg, a flat dose of about 200 mg, a flat dose of about 250 mg, or a flat dose of about 300 mg per administration.

In some embodiments, the treatment dose is a flat dose of between about 150 mg to about 250 mg, for example a flat dose of about 150 mg, a flat dose of about 200 mg, or a flat dose of about 250 mg per administration.

In some embodiments, the treatment dose is a flat dose of about 150 mg to about 200 mg, for example a flat dose of about 150 mg, or a flat dose of about 200 mg per administration.

In a particular embodiment, the treatment dose is a flat dose of about 150 mg per administration.

In some embodiments, the treatment dose is a flat dose of at least about 200 mg per administration.

In some embodiments, the treatment dose is a flat dose of between about 200 mg to about 900 mg, for example a flat dose of about 200 mg, a flat dose of about 250 mg, a flat dose of about 300 mg, a flat dose of about 400 mg, a flat dose of about 500 mg, a flat dose of about 600 mg, a flat dose of about 700 mg, a flat dose of about 800 mg or a flat dose of about 900 mg per administration.

In some embodiments, the treatment dose is a flat dose of between about 200 mg to about 600 mg, for example a flat dose of about 200 mg, a flat dose of about 250 mg, a flat dose of about 300 mg, a flat dose of about 400 mg, a flat dose of about 500 mg, or a flat dose of about 600 mg per administration.

In some embodiments, the treatment dose is a flat dose of between about 200 mg to about 300 mg, for example a flat dose of about 200 mg, a flat dose of about 250 mg, or a flat dose of about 300 mg per administration.

In some embodiments, the treatment dose is a flat dose of between about 200 mg to about 250 mg, for example a flat dose of about 200 mg, or a flat dose of about 250 mg per administration.

In a particular embodiment, the treatment dose is a flat dose of about 200 mg per administration.

In some embodiments, the treatment dose is a flat dose of at least about 250 mg per administration.

In some embodiments, the treatment dose is a flat dose of between about 250 mg to about 900 mg, for example a flat dose of about 250 mg, a flat dose of about 300 mg, a flat dose of about 400 mg, a flat dose of about 500 mg, a flat dose of about 600 mg, a flat dose of about 700 mg, a flat dose of about 800 mg or a flat dose of about 900 mg per administration.

In some embodiments, the treatment dose is a flat dose of between about 250 mg to about 600 mg, for example a flat dose of about 250 mg, a flat dose of about 300 mg, a flat dose of about 400 mg, a flat dose of about 500 mg, or a flat dose of about 600 mg per administration.

In some embodiments, the treatment dose is a flat dose of between about 250 mg to about 300 mg, for example a flat dose of about 250 mg, or a flat dose of about 300 mg per administration.

In a particular embodiment, the treatment dose is a flat dose of about 250 mg per administration.

In some embodiments, the treatment dose is a flat dose of at least about 300 mg per administration.

In some embodiments, the treatment dose is a flat dose of between about 300 to about 900 mg per administration, for example a flat dose of about 300 mg, a flat dose of about 400 mg, a flat dose of about 500 mg, a flat dose of about 600 mg, a flat dose of about 700 mg, a flat dose of about 800 mg or a flat dose of about 900 mg per administration.

In some embodiments, the treatment dose is a flat dose of between about 300 mg to about 600 mg, for example a flat dose of about 300 mg, a flat dose of about 400 mg, a flat dose of about 500 mg, or a flat dose of about 600 mg per administration.

In a particular embodiment, the treatment dose is a flat dose of about 300 mg per administration.

In some embodiments, the treatment dose is a flat dose of at least about 600 mg per administration.

In some embodiments, the treatment dose is a flat dose of between about 600 to about 900 mg per administration, for example a flat dose of about 600 mg, a flat dose of about 700 mg, a flat dose of about 800 mg or a flat dose of about 900 mg per administration.

In a particular embodiment, the treatment dose is a flat dose of about 600 mg per administration.

In a particular embodiment, the treatment dose is a flat dose of about 900 mg per administration.

The treatment dose of the isolated anti-hK2/anti-CD3 bispecific antibody or bispecific fragment thereof may be a weight-based dose. Therefore, in some embodiments of the invention, the treatment dose is a weight-based dose. Such dosages can, for example, be based on the mg/kg dosages provided above according to the following: dose (mg/kg)×body weight (e.g., 50-100 kg).

### Administration Frequency

In some embodiments, the treatment dose of the isolated anti-hK2/anti-CD3 bispecific antibody or bispecific fragment thereof is administered every one to six weeks, for example, once a week, once every two weeks, once every three weeks, once every four weeks, once every five week or once every six weeks.

In a particular embodiment, the treatment dose is administered once every three weeks.

In a particular embodiment, the treatment dose is administered once every six weeks.

In a particular embodiment, the treatment dose is administered once every 42 days+/−3 days, preferably once every 42 days.

In some embodiments, the treatment dose is at least about 75 mg per administration (for example a flat dose of at least about 75 mg per administration) and is administered once every three weeks.

In some embodiments, the treatment dose is between about 75 mg and about 900 mg per administration (for example a flat dose of between about 75 mg and about 900 mg per administration) and is administered once every three weeks. In some embodiments, the treatment dose is between about 75 mg and about 600 mg per administration (for example a flat dose of between about 75 mg and about 600 mg per administration) and is administered once every three weeks. In some embodiments, the treatment dose is between about 75 mg and about 300 mg per administration (for example a flat dose of between about 75 mg and 300 mg per administration) and is administered once every three weeks. In some embodiments, the treatment dose is between about 75 mg and about 250 mg per administration (for example a flat dose of between about 75 mg and about 250 mg per administration) and is administered once every three weeks. In some embodiments, the treatment dose is between about 75 mg and about 200 mg per administration (for example a flat dose of between about 75 mg and about 200 mg per administration) and is administered once every three weeks. In some embodiments, the treatment dose is between about 75 mg and about 150 mg per administration (for example a flat dose of between about 75 mg and about 150 mg per administration) and is administered once every three weeks.

In a particular embodiment, the treatment dose is about 75 mg per administration (for example a flat dose of about 75 mg per administration) administered once every three weeks.

In some embodiments, the treatment dose is at least about 150 mg per administration (for example a flat dose of about 150 mg per administration) administered once every three weeks.

In some embodiments, the treatment dose is between about 150 mg to about 900 mg per administration (for example a flat dose of between about 150 mg and about 900 mg per administration) and is administered once every three weeks. In some embodiments, the treatment dose is between about 150 mg to about 600 mg per administration (for example a flat dose of between about 150 mg and about 600 mg per administration) and is administered once every three weeks. In some embodiments, the treatment dose is between about 150 mg to about 300 mg per administration (for example a flat dose of between about 150 mg and about 300 mg per administration) and is administered once every three weeks. In some embodiments, the treatment dose is between about 150 mg to about 250 mg per administration (for example a flat dose of between about 150 mg and about 250 mg per administration) and is administered once every three weeks. In some embodiments, the treatment dose is about 150 mg to about 200 mg per administration (for example a flat dose of between about 150 mg and about 200 mg per administration) and is administered once every three weeks.

In a particular embodiment, the treatment dose is about 150 mg per administration (for example a flat dose of about 150 mg per administration) administered once every three weeks.

In some embodiments, the treatment dose is at least about 200 mg per administration (for example a flat dose of at least about 200 mg per administration) administered once every three weeks.

In some embodiments, the treatment dose is between about 200 mg to about 900 mg per administration (for example a flat dose of between about 200 mg and about 900 mg per administration) and is administered once every three weeks. In some embodiments, the treatment dose is between about 200 mg to about 600 mg per administration (for example a flat dose of between about 200 mg and about 600 mg per administration) and is administered once every three weeks. In some embodiments, the treatment dose is between about 200 mg to about 300 mg per administration (for example a flat dose of between about 200 mg and about 300 mg per administration) and is administered once every three weeks.

In some embodiments, the treatment dose is between about 200 mg to about 250 mg per administration (for example a flat dose of between about 200 mg and about 250 mg per administration) and is administered once every three weeks.

In a particular embodiment, the treatment dose is about 200 mg per administration (for example a flat dose of about 200 mg per administration) administered once every three weeks.

In some embodiments, the treatment dose is at least about 250 mg per administration (for example a flat dose of at least about 250 mg per administration) administered once every three weeks.

In some embodiments, the treatment dose is between about 250 mg to about 900 mg per administration (for example a flat dose of between about 250 mg and about 900 mg per administration) and is administered once every three weeks. In some embodiments, the treatment dose is between about 250 mg to about 600 mg (for example a flat dose of between about 250 mg and about 600 mg per administration) and is administered once every three weeks. In some embodiments, the treatment dose is between about 250 mg to about 300 mg (for example a flat dose of between about 250 mg and about 300 mg per administration) and is administered once every three weeks.

In a particular embodiment, the treatment dose is about 250 mg per administration (for example a flat dose of about 250 mg per administration) administered once every three weeks

In some embodiments, the treatment dose is at least about 300 mg per administration (for example a flat dose of about 300 mg per administration) administered once every three weeks.

In some embodiments, the treatment dose is between about 300 mg to about 900 mg per administration (for example between about 300 mg to about 900 mg per administration) administered once every three weeks. In some embodiments, the treatment dose is between about 300 mg to about 600 mg per administration (for example a flat dose of between about 300 mg to about 600 mg per administration) administered once every three weeks.

In a particular embodiment, the treatment dose is about 300 mg per administration (for example a flat dose of about 300 mg per administration) administered once every three weeks.

In some embodiments, the treatment dose is at least about 300 mg per administration (for example a flat dose of about 300 mg per administration) administered once every six weeks.

In some embodiments, the treatment dose is between about 300 mg to about 900 mg per administration (for example between about 300 mg to about 900 mg per administration) administered once every six weeks. In some embodiments, the treatment dose is between about 300 mg to about 600 mg per administration (for example a flat dose of between about 300 mg to about 600 mg per administration) administered once every six weeks.

In a particular embodiment, the treatment dose is about 300 mg per administration (for example a flat dose of about 300 mg per administration) administered once every six weeks.

In some embodiments, the treatment dose is at about 300 mg per administration (for example a flat dose of about 300 mg per administration) administered once every 42 days+/−3 days, preferably once every 42 days.

In some embodiments, the treatment dose is at least about 600 mg per administration (for example a flat dose of at least about 600 mg per administration) and is administered once every six weeks.

In some embodiments, the treatment dose is between about 600 mg to about 900 mg per administration (for example a flat dose between about 600 mg to about 900 mg per administration) and is administered once every six weeks.

In a particular embodiment, the treatment dose is about 600 mg per administration (for example a flat dose of about 600 mg per administration) and is administered once every six weeks.

In a particular embodiment, the treatment dose is about 900 mg per administration (for example a flat dose of about 900 mg per administration) and is administered once every six weeks.

The methods of the invention comprise administering a treatment dose of an isolated anti-hK2/anti-CD3 bispecific antibody or a bispecific fragment thereof, as defined herein, to the subject to treat prostate cancer. One or more treatment doses may be administered to the subject and typically several treatment doses are administered to the subject in the course of the treatment. The administration of several treatment doses of the isolated anti-hK2/anti-CD3 bispecific antibody or bispecific fragment thereof may be defined according to a number of cycles of administration. For instance, a treatment dose may be administered once every three weeks over a twelve-week period. In this case, the treatment dose is administered four times in a twelve-week period and the treatment dose is therefore administered for four cycles (i.e. there are four three week cycles). As another example, a treatment dose may be administered one every six weeks over a twelve week period. In this case, the treatment dose is administered twice in a twelve week period and the treatment dose is therefore administered for two cycles (i.e. there are two six week cycles).

Accordingly, the treatment dose may be administered in one or more cycles. In some embodiments, the treatment dose is administered for at least two cycles, for example, two, three, four, five, ten, fifteen, twenty or more cycles. Any of the treatment doses described elsewhere herein may be administered in the one or more cycles (for example, two, three, four, five, ten, fifteen, twenty or more cycles). The treatment dose that is administered in each cycle may be administered at the same dose in each cycle.

In some embodiments, the treatment dose of the isolated anti-hK2/anti-CD3 bispecific antibody or bispecific fragment thereof is administered every one to six weeks, for example, once a week, once every two weeks, once every three weeks, once every four weeks, once every five weeks or once every six weeks for at least two cycles (for example, two, three, four, five, ten, fifteen, twenty or more cycles).

In a particular embodiment, the treatment dose is administered once every three weeks for at least two cycles (for example, two, three, four, five, ten, fifteen, twenty or more cycles).

In some embodiments, the treatment dose is administered once every three weeks for at least two cycles. In some embodiments, the treatment dose is administered once every three weeks for at least three cycles. In some embodiments, the treatment dose is administered once every three weeks for at least four cycles. In some embodiments, the treatment dose is administered once every three weeks for at least five cycles. In some embodiments, the treatment dose is administered once every three weeks for at least ten cycles. In some embodiments, the treatment dose is administered once every three weeks for at least fifteen cycles. In some embodiments, the treatment dose is administered once every three weeks for at least twenty cycles.

In some embodiments, the treatment dose is administered once every six weeks for at least two cycles. In some embodiments, the treatment dose is administered once every six weeks for at least three cycles. In some embodiments, the treatment dose is administered once every six weeks for at least four cycles. In some embodiments, the treatment dose is administered once every six weeks for at least five cycles. In some embodiments, the treatment dose is administered once every six weeks for at least ten cycles. In some embodiments, the treatment dose is administered once every six weeks for at least fifteen cycles. In some embodiments, the treatment dose is administered once every six weeks for at least twenty or more cycles.

In some embodiments, the treatment dose is administered for at least a year.

In some embodiments, the treatment dose is administered throughout the lifespan of the patient.

### Step-Up Dosing

The isolated anti-hK2/anti-CD3 bispecific antibody or bispecific fragment thereof may be administered with or without step-up dosing. Administration of step-up doses prior to treatment doses may prevent or lessen certain adverse events or side-effects that can be associated with administration of bispecific CD3 antibodies, such as cytokine release syndrome (CRS). The step-up doses are not higher than the treatment dose and thus may function as priming doses that prevent or lessen adverse events.

Accordingly, in some embodiments, the method comprises administering to the human subject one or more one step-up doses of the isolated anti-hK2/anti-CD3 bispecific antibody or bispecific fragment thereof, wherein the one or more step-up doses are administered prior to the treatment dose of the isolated anti-hK2/anti-CD3 bispecific antibody or bispecific fragment thereof and wherein the one or more step-up doses are not higher than the treatment dose.

In some embodiments, one step-up dose of the isolated anti-hK2/anti-CD3 bispecific antibody or bispecific fragment thereof is administered.

In some embodiments, two step-up doses of the isolated anti-hK2/anti-CD3 bispecific antibody or bispecific fragment thereof are administered.

In some embodiments, the method comprises administering to the subject a step-up dose of between about 2 mg to about 5 mg per administration. In certain embodiments, the step-up dose is about 2 mg per administration. In other embodiments, the step-up dose is about 2.5 mg per administration. In other embodiments, the step-up dose is about 3 mg per administration. In other embodiments, the step-up dose is about 3.5 mg per administration. In other embodiments, the step-up dose is about 4 mg per administration. In other embodiments, the step-up dose is about 4.5 mg per administration. In other embodiments, the step-up dose is about 5 mg per administration.

In a particular embodiment, the step-up dose is about 3.5 mg per administration.

In some embodiments, the method comprises administering to the subject a step-up dose of between about 5 mg to about 15 mg per administration. In certain embodiments, the step-up dose is about 5 mg per administration. In other embodiments, the step-up dose is about 10 mg per administration. In other embodiments, the step-up dose is about 15 mg per administration.

In a particular embodiment, the step-up dose is about 10 mg per administration.

In some embodiments, the method comprises administering to the subject a step-up dose of between about 15 mg to about 25 mg per administration. In certain embodiments, the step-up dose is about 15 mg per administration. In other embodiments, the step-up dose is about 16 mg per administration. In other embodiments, the step-up dose is about 17 mg per administration. In other embodiments, the step-up dose is about 18 mg per administration. In other embodiments, the step-up dose is about 19 mg per administration. In other embodiments, the step-up dose is about 20 mg per administration. In other embodiments, the step-up dose is about 21 mg per administration. In other embodiments, the step-up dose is about 22 mg per administration. In other embodiments, the step-up dose is about 23 mg per administration. In other embodiments, the step-up dose is about 24 mg per administration. In other embodiments, the step-up dose is about 25 mg per administration.

In a particular embodiment, the step-up dose is about 18 mg per administration.

In some embodiments, the method comprises administering to the subject a step-up dose of between about 200 mg to about 400 mg per administration. In certain embodiments, the step-up dose is about 200 mg per administration. In other embodiments, the step-up dose is about 300 mg per administration. In other embodiments, the step-up dose is about 400 mg per administration.

In a particular embodiment, the step-up dose is about 300 mg per administration.

The step-up dose of the isolated anti-hK2/anti-CD3 bispecific antibody or bispecific fragment thereof may be administered as a flat dose.

In some embodiments, the method comprises administering to the subject a step-up dose at a flat dose of between about 2 mg to about 5 mg per administration. In certain embodiments, the step-up dose is a flat dose of about 2 mg per administration. In other embodiments, the step-up dose is a flat dose of about 2.5 mg per administration. In other embodiments, the step-up dose is a flat dose of about 3 mg per administration. In other embodiments, the step-up dose is a flat dose of about 3.5 mg per administration. In other embodiments, the step-up dose is a flat dose of about 4 mg per administration. In other embodiments, the step-up dose is a flat dose of about 4.5 mg per administration. In other embodiments, the step-up dose is a flat dose of about 5 mg per administration.

In a particular embodiment, the step-up dose is a flat dose of about 3.5 mg per administration.

In some embodiments, the method comprises administering to the subject a step-up dose at a flat dose of between about 5 mg to about 15 mg per administration. In certain embodiments, the step-up dose is a flat dose of about 5 mg per administration. In other embodiments, the step-up dose is a flat dose of about 10 mg per administration. In other embodiments, the step-up dose is about a flat dose of 15 mg per administration.

In a particular embodiment, the step-up dose is a flat dose of about 10 mg per administration.

In some embodiments, the method comprises administering to the subject a step-up dose at a flat dose of between about 15 mg to about 25 mg per administration. In certain embodiments, the step-up dose is a flat dose of about 15 mg per administration. In other embodiments, the step-up dose is a flat dose of about 16 mg per administration. In other embodiments, the step-up dose is a flat dose of about 17 mg per administration. In other embodiments, the step-up dose is a flat dose of about 18 mg per administration. In other embodiments, the step-up dose is a flat dose of about 19 mg per administration. In other embodiments, the step-up dose is a flat dose of about 20 mg per administration. In other embodiments, the step-up dose is a flat dose of about 21 mg per administration. In other embodiments, the step-up dose is a flat dose of about 22 mg per administration. In other embodiments, the step-up dose is a flat dose of about 23 mg per administration. In other embodiments, the step-up dose is a flat dose of about 24 mg per administration. In other embodiments, the step-up dose is a flat dose of about 25 mg per administration.

In a particular embodiment, the step-up dose is a flat dose of about 18 mg per administration.

In some embodiments, the method comprises administering to the subject a step-up dose at a flat dose of between about 200 mg to about 400 mg per administration. In certain embodiments, the step-up dose is a flat dose of about 200 mg per administration. In other embodiments, the step-up dose is a flat dose of about 300 mg per administration. In other embodiments, the step-up dose is a flat dose of about 400 mg per administration.

In a particular embodiment, the step-up dose is a flat dose of about 300 mg per administration.

The step-up dose of the isolated anti-hK2/anti-CD3 bispecific antibody or bispecific fragment thereof may be a weight-based dose. Therefore, in some embodiments of the invention, the step-up dose is a weight-based dose. Such dosages can, for example, be based on the mg/kg dosages provided above according to the following: dose (mg/kg)×body weight (e.g., 50-100 kg).

In some embodiments, the step-up dose of the isolated anti-hK2/anti-CD3 bispecific antibody or bispecific fragment thereof is administered 3 days to 8 days prior to the administration of the treatment dose. Throughout the application, in the context of the timing for an administration, the expressions “X days to Y days” and “a day within X days to Y days” prior to or after a stated event shall have the same meaning, which encompasses the administration on a day of X days, Y days, or any days between X days and Y days, prior to or after the stated event. For example, if a stated event (e.g., administration of the treatment dose) occurs on Day 1, an administration 3 days to 8 days before the stated event can be an administration on a day that is 3 days, 4 days, 5 days, 6 days, 7 days or 8 days before Day 1.

In certain embodiments, the step-up dose is administered 3 days prior to the administration of the treatment dose. In other embodiments, the step-up dose is administered 4 days prior to the administration of the treatment dose. In other embodiments, the step-up dose is administered 5 days prior to the administration of the treatment dose. In other embodiments, the step-up dose is administered 6 days prior to the administration of the treatment dose. In other embodiments, the step-up dose is administered 7 days prior to the administration of the treatment dose. In other embodiments, the step-up dose is administered 8 days prior to the administration of the treatment dose.

In a particular embodiment, the step-up dose of the isolated anti-hK2/anti-CD3 bispecific antibody or bispecific fragment thereof is administered 3 days prior to the administration of the treatment dose.

In some embodiments, the method comprises administering to the subject a step-up dose of between about 2 mg to about 5 mg per administration (for example a flat dose of between about 2 mg to about 5 mg per administration) and the administration of the step-up dose is 3 days prior to the administration of the treatment dose.

In a particular embodiment, the step-up dose is about 3.5 mg per administration (for example a flat dose of about 3.5 mg per administration) and the administration of the step-up dose is 3 days prior to the administration of the treatment dose.

In some embodiments, the method comprises administering to the subject a step-up dose of between about 5 mg to about 15 mg per administration (for example a flat dose of between about 5 mg to about 15 mg per administration) and the administration of the step-up dose is 3 days prior to the administration of the treatment dose.

In a particular embodiment, the step-up dose is about 10 mg per administration (for example a flat dose of about 10 mg per administration) and the administration of the step-up dose is 3 days prior to the administration of the treatment dose.

In some embodiments, the method comprises administering to the subject a step-up dose of between about 15 mg to about 25 mg per administration (for example a flat dose of between about 15 mg to about 25 mg per administration) and the administration of the step-up dose is 3 days prior to the administration of the treatment dose.

In a particular embodiment, the step-up dose is about 18 mg per administration (for example a flat dose of about 18 mg per administration) and the administration of the step-up dose is 3 days prior to the administration of the treatment dose.

In some embodiments, the method comprises administering to the subject a step-up dose of between about 200 mg to about 400 mg per administration (for example a flat dose of between about 200 mg to about 400 mg per administration) and the administration of the step-up dose is 3 days prior to the administration of the treatment dose.

In a particular embodiment, the step-up dose is about 300 mg per administration (for example a flat dose of about 300 mg per administration) and the administration of the step-up dose is 3 days prior to the administration of the treatment dose.

In a particular embodiment, the step-up dose of the isolated anti-hK2/anti-CD3 bispecific antibody or bispecific fragment thereof is administered 7 days prior to the administration of the treatment dose.

In some embodiments, the method comprises administering to the subject a step-up dose of between about 2 mg to about 5 mg per administration (for example a flat dose of between about 2 mg to about 5 mg per administration) and the administration of the step-up dose is 7 days prior to the administration of the treatment dose. In a particular embodiment, the step-up dose is about 3.5 mg per administration (for example a flat dose of about 3.5 mg per administration) and the administration of the step-up dose is 7 days prior to the administration of the treatment dose.

In some embodiments, the method comprises administering to the subject a step-up dose of between about 5 mg to about 15 mg per administration (for example a flat dose of between about 5 mg to about 15 mg per administration) and the administration of the step-up dose is 7 days prior to the administration of the treatment dose. In a particular embodiment, the step-up dose is about 10 mg per administration (for example a flat dose of about 10 mg per administration) and the administration of the step-up dose is 7 days prior to the administration of the treatment dose.

In some embodiments, the method comprises administering to the subject a step-up dose of between about 15 mg to about 25 mg per administration (for example a flat dose of between about 15 mg to about 25 mg per administration) and the administration of the step-up dose is 7 days prior to the administration of the treatment dose. In a particular embodiment, the step-up dose is about 18 mg per administration (for example a flat dose of 18 mg per administration) and the administration of the step-up dose is 7 days prior to the administration of the treatment dose.

In some embodiments, the method comprises administering to the subject a step-up dose of between about 200 mg to about 400 mg per administration (for example a flat dose of between about 200 mg to about 400 mg per administration) and the administration of the step-up dose is 7 days prior to the administration of the treatment dose. In a particular embodiment, the step-up dose is about 300 mg per administration (for example a flat dose of 300 mg per administration) and the administration of the step-up dose is 7 days prior to the administration of the treatment dose.

In any of the above embodiments the method may comprise administering to the subject a step-up dose of the isolated anti-hK2/anti-CD3 bispecific antibody or bispecific fragment thereof; and a treatment dose of the isolated anti-hK2/anti-CD3 bispecific antibody or bispecific fragment thereof, wherein the step-up dose is administered once.

The inventors have found that administration by intravenous infusion may be advantageous in reducing the likelihood of adverse events following administration of bispecific antibodies. The step-up doses may therefore be administered by intravenous infusion. Accordingly, in some embodiments the step-up dose of the isolated anti-hK2/anti-CD3 bispecific antibody or bispecific fragment thereof is administered by intravenous (IV) infusion.

An intravenous infusion of the isolated anti-hK2/anti-CD3 bispecific antibody or bispecific fragment thereof may be administered over several minutes to several hours. The inventors have found that administration of the step-up dose of an isolated anti-hK2/anti-CD3 bispecific antibody or bispecific fragment thereof over a period of one hour followed by administration of subsequent infusions of the treatment dose over a period of 30 minutes are suitable timings for administration. Accordingly, in some embodiments, the step-up dose is administered by intravenous (IV) infusion over a period of at least one hour. In certain such embodiments, the step-up dose is administered by intravenous (IV) infusion over a period of about one hour.

In some embodiments, the step-up dose is administered by intravenous infusion over a period of at least one hour (for example over a period of about one hour) and any subsequent treatment dose is administered by intravenous infusion over a period of at least 30 minutes (for example over a period of about 30 minutes).

The methods of the invention may also involve administering two step-up doses prior to the treatment dose. Typically, the second step-up dose is greater than the first step-up dose and the treatment dose is greater than the second step-up dose.

Accordingly, in some embodiments the method comprises administering to the subject a first step-up dose and a second step-up dose of the isolated anti-hK2/anti-CD3 bispecific antibody or bispecific fragment thereof, wherein the first step-up dose is administered prior to the second step-up dose, and the second step-up dose is administered prior to the treatment dose.

In some embodiments, the second step-up dose is greater than the first step-up dose.

In some embodiments, the method comprises administering to the subject a first step-up dose of between about 2 mg to about 5 mg per administration. In certain embodiments, the first step-up dose is about 2 mg per administration. In other embodiments, the first step-up dose is about 2.5 mg per administration. In other embodiments, the first step-up dose is about 3 mg per administration. In other embodiments, the first step-up dose is about 3.5 mg per administration. In other embodiments, the first step-up dose is about 4 mg per administration. In other embodiments, the first step-up dose is about 4.5 mg per administration. In other embodiments, the first step-up dose is about 5 mg per administration.

In a particular embodiment, the first step-up dose is about 3.5 mg per administration.

In some embodiments, the method comprises administering to the subject a first step-up dose of between about 5 mg to about 15 mg per administration. In certain embodiments, the first step-up dose is about 5 mg per administration. In other embodiments, the first step-up dose is about 10 mg per administration. In other embodiments, the first step-up dose is about 15 mg per administration.

In a particular embodiment, the first step-up dose is about 10 mg per administration.

In some embodiments, the method comprises administering to the subject a second step-up dose of between about 5 mg to about 15 mg per administration. In certain embodiments, the second step-up dose is about 5 mg per administration. In other embodiments, the second step-up dose is about 10 mg per administration. In other embodiments, the second step-up dose is about 15 mg per administration.

In a particular embodiment, the second step-up dose is about 10 mg per administration.

In some embodiments, the method comprises administering to the subject a second step-up dose of between about 15 mg to about 25 mg per administration. In certain embodiments, the second step-up dose is about 15 mg per administration. In other embodiments, the second step-up dose is about 16 mg per administration. In other embodiments, the second step-up dose is about 17 mg per administration. In other embodiments, the second step-up dose is about 18 mg per administration. In other embodiments, the second step-up dose is about 19 mg per administration. In other embodiments, the second step-up dose is about 20 mg per administration. In other embodiments, the second step-up dose is about 21 mg per administration. In other embodiments, the second step-up dose is about 22 mg per administration. In other embodiments, the second step-up dose is about 23 mg per administration. In other embodiments, the second step-up dose is about 24 mg per administration. In other embodiments, the second step-up dose is about 25 mg per administration.

In a particular embodiment, the second step-up dose is about 18 mg per administration.

In some embodiments, the method comprises administering to the subject a second step-up dose of between about 200 mg to about 400 mg per administration. In certain embodiments, the second step-up dose is about 200 mg per administration. In other embodiments the second step-up dose is about 300 mg per administration. In other embodiments the second step-up dose is about 400 mg per administration.

In a particular embodiment, the second step-up dose is about 300 mg per administration.

The first step-up dose and the second step-up dose of the isolated anti-hK2/anti-CD3 bispecific antibody or bispecific fragment thereof may be a flat dose.

Accordingly, in some embodiments, the method comprises administering to the subject a first step-up dose at a flat dose of between about 2 mg to about 5 mg per administration. In certain embodiments, the first step-up dose is a flat dose of about 2 mg per administration. In other embodiments, the first step-up dose is a flat dose of about 2.5 mg per administration. In other embodiments, the first step-up dose is a flat dose of about 3 mg per administration. In other embodiments, the first step-up dose is a flat dose of about 3.5 mg per administration. In other embodiments, the first step-up dose is a flat dose of about 4 mg per administration. In other embodiments, the first step-up dose is a flat dose of about 4.5 mg per administration. In other embodiments, the first step-up dose is a flat dose of about 5 mg per administration.

In a particular embodiment, the first step-up dose is a flat dose of about 3.5 mg per administration.

In some embodiments, the method comprises administering to the subject a first step-up dose at a flat dose of between about 5 mg to about 15 mg per administration. In certain embodiments, the first step-up dose is a flat dose of about 5 mg per administration. In other embodiments, the first step-up dose is a flat dose of about 10 mg per administration. In other embodiments, the first step-up dose is a flat dose of about 15 mg per administration.

In a particular embodiment, the first step-up dose is a flat dose of about 10 mg per administration.

In some embodiments, the method comprises administering to the subject a second step-up dose at a flat dose of between about 5 mg to about 15 mg per administration. In certain embodiments, the second step-up dose is a flat dose of about 5 mg per administration. In other embodiments, the second step-up dose is a flat dose of about 10 mg per administration. In other embodiments, the second step-up dose is a flat dose of about 15 mg per administration.

In a particular embodiment, the second step-up dose is a flat dose of about 10 mg per administration.

In some embodiments, the method comprises administering to the subject a second step-up dose at a flat dose of between about 15 mg to about 25 mg per administration. In certain embodiments, the second step-up dose is a flat dose of about 15 mg per administration. In other embodiments, the second step-up dose is a flat dose of about 16 mg per administration. In other embodiments, the second step-up dose is a flat dose of about 17 mg per administration. In other embodiments, the second step-up dose is a flat dose of about 18 mg per administration. In other embodiments, the second step-up dose is a flat dose of about 19 mg per administration. In other embodiments, the second step-up dose is a flat dose of about 20 mg per administration. In other embodiments, the second step-up dose is a flat dose of about 21 mg per administration. In other embodiments, the second step-up dose is a flat dose of about 22 mg per administration. In other embodiments, the second step-up dose is a flat dose of about 23 mg per administration. In other embodiments, the second step-up dose is a flat dose of about 24 mg per administration. In other embodiments, the second step-up dose is a flat dose of about 25 mg per administration.

In a particular embodiment, the second step-up dose is a flat dose of about 18 mg per administration.

In some embodiments, the method comprises administering to the subject a second step-up dose at a flat dose of between about 200 mg to about 400 mg per administration. In certain embodiments, the second step-up dose is a flat dose of about 200 mg per administration. In other embodiments the second step-up dose is a flat dose of about 300 mg per administration. In other embodiments the second step-up dose is a flat dose of about 400 mg per administration.

In a particular embodiment, the second step-up dose is a flat dose of about 300 mg per administration.

The first step-up dose and the second step-up dose of the isolated anti-hK2/anti-CD3 bispecific antibody or bispecific fragment thereof may be a weight-based dose. Therefore, in some embodiments of the invention, the first step-up dose and the second step-up dose are weight-based doses. Such dosages can, for example, be based on the mg/kg dosages provided above according to the following: dose (mg/kg)×body weight (e.g., 50-100 kg).

In some embodiments, the first step-up dose of the isolated anti-hK2/anti-CD3 bispecific antibody or bispecific fragment thereof is administered 3 days to 8 days prior to the administration of the second step-up dose. In certain embodiments, the first step-up dose is administered 3 days prior to the administration of the second step-up dose. In other embodiments, the first step-up dose is administered 4 days prior to the administration of the second step-up dose. In other embodiments, the first step-up dose is administered 5 days prior to the administration of the second step-up dose. In other embodiments, the first step-up dose is administered 6 days prior to the administration of the second step-up dose. In other embodiments, the first step-up dose is administered 7 days prior to the administration of the second step-up dose. In other embodiments, the first step-up dose is administered 8 days prior to the administration of the second step-up dose.

In a particular embodiment, the first step-up dose of the isolated anti-hK2/anti-CD3 bispecific antibody or bispecific fragment thereof is administered 3 days prior to the administration of the second step-up dose.

In some embodiments, the method comprises administering to the subject a first step-up dose of between about 2 mg to about 5 mg per administration (for example a flat dose of about 2 mg to about 5 mg per administration) and the first step-up dose is administered 3 days prior to the administration of the second step-up dose. In a particular embodiment, the first step-up dose is about 3.5 mg per administration (for example a flat dose of about 3.5 mg per administration) and the first step-up dose is administered 3 days prior to the administration of the second step-up dose.

In some embodiments, the method comprises administering to the subject a first step-up dose of between about 5 mg to about 15 mg per administration (for example a flat dose of between about 5 mg to about 15 mg per administration) and the first step-up dose is administered 3 days prior to the administration of the second step-up dose. In a particular embodiment, the first step-up dose is about 10 mg per administration (for example a flat dose of about 10 mg per administration) and the first step-up dose is administered 3 days prior to the administration of the second step-up dose.

In a particular embodiment, the first step-up dose of the isolated anti-hK2/anti-CD3 bispecific antibody or bispecific fragment thereof is administered 7 days prior to the administration of the second step-up dose.

In some embodiments, the method comprises administering to the subject a first step-up dose of between about 2 mg to about 5 mg per administration (for example a flat dose of between about 2 mg to about 5 mg per administration) and the first step-up dose is administered 7 days prior to the administration of the second step-up dose. In a particular embodiment, the first step-up dose is about 3.5 mg per administration (for example a flat dose of about 3.5 mg per administration) and the first step-up dose is administered 7 days prior to the administration of the second step-up dose.

In some embodiments, the method comprises administering to the subject a first step-up dose of between about 5 mg to about 15 mg per administration (for example a flat dose of between about 5 mg to about 15 mg per administration) and the first step-up dose is administered 7 days prior to the administration of the second step-up dose. In a particular embodiment, the first step-up dose is about 10 mg per administration (for example a flat dose of about 10 mg per administration) and the first step-up dose is administered 7 days prior to the administration of the second step-up dose.

In some embodiments, the second step-up dose of the isolated anti-hK2/anti-CD3 bispecific antibody or bispecific fragment thereof is administered 3 days to 8 days prior to the administration of the treatment dose. In certain embodiments, the second step-up dose is administered 3 days prior to the administration of the treatment dose. In other embodiments, the second step-up dose is administered 4 days prior to the administration of the treatment dose. In other embodiments, the second step-up dose is administered 5 days prior to the administration of the treatment dose. In other embodiments, the second step-up dose is administered 6 days prior to the administration of the treatment dose. In other embodiments, the second step-up dose is administered 7 days prior to the administration of the treatment dose. In other embodiments, the second step-up dose is administered 8 days prior to the administration of the treatment dose.

In a particular embodiment, the second step-up dose of the isolated anti-hK2/anti-CD3 bispecific antibody or bispecific fragment thereof is administered 3 days prior to the administration of the treatment dose.

In some embodiments, the method comprises administering to the subject a second step-up dose of between about 5 mg to about 15 mg per administration (for example a flat dose of between about 5 mg to about 15 mg per administration) and the second step-up dose is administered 3 days prior to the administration of the treatment dose. In a particular embodiment, the second step-up dose is about 10 mg per administration (for example a flat dose of about 10 mg per administration) and the second step-up dose is administered 3 days prior to the administration of the treatment dose.

In some embodiments, the method comprises administering to the subject a second step-up dose of between about 15 mg to about 25 mg per administration (for example a flat dose of between about 15 mg to about 25 mg per administration) and the second step-up dose is administered 3 days prior to the administration of the treatment dose. In a particular embodiment, the second step-up dose is about 18 mg per administration (for example a flat dose of about 18 mg per administration) and the second step-up dose is administered 3 days prior to the administration of the treatment dose.

In some embodiments, the method comprises administering to the subject a second step-up dose of between about 200 mg to about 400 mg per administration (for example a flat dose of between about 200 mg to about 400 mg per administration) and the second step-up dose is administered 3 days prior to the administration of the treatment dose. In a particular embodiment, the second step-up dose is about 300 mg per administration (for example a flat dose of about 300 mg per administration) and the second step-up dose is administered 3 days prior to the administration of the treatment dose.

In a particular embodiment, the second step-up dose of the isolated anti-hK2/anti-CD3 bispecific antibody or bispecific fragment thereof is administered 7 days prior to the administration of the treatment dose.

In some embodiments, the method comprises administering to the subject a second step-up dose of between about 5 mg to about 15 mg per administration (for example a flat dose of between about 5 mg to about 15 mg per administration) and the second step-up dose is administered 7 days prior to the administration of the treatment dose. In a particular embodiment, the second step-up dose is about 10 mg per administration (for example a flat dose of about 10 mg per administration) and the second step-up dose is administered 7 days prior to the administration of the treatment dose.

In some embodiments, the method comprises administering to the subject a second step-up dose of between about 15 mg to about 25 mg per administration (for example a flat dose of between about 15 mg to about 25 mg per administration) and the second step-up dose is administered 7 days prior to the administration of the treatment dose. In a particular embodiment, the second step-up dose is about 18 mg per administration (for example a flat dose of about 18 mg per administration) and the second step-up dose is administered 7 days prior to the administration of the treatment dose.

In some embodiments, the method comprises administering to the subject a second step-up dose of between about 200 mg to about 400 mg per administration (for example a flat dose of between about 200 mg to about 400 mg per administration) and the second step-up dose is administered 7 days prior to the administration of the treatment dose. In a particular embodiment, the second step-up dose is about 300 mg per administration (for example a flat dose of about 300 mg per administration) and the second step-up dose is administered 7 days prior to the administration of the treatment dose.

In embodiments that involve the administration of two step-up doses, the method may comprise administering to the subject: a first step-up dose of the isolated anti-hK2/anti-CD3 bispecific antibody or bispecific fragment thereof; a second step-up dose of the isolated anti-hK2/anti-CD3 bispecific antibody or bispecific fragment thereof; and a treatment dose of the isolated anti-hK2/anti-CD3 bispecific antibody or bispecific fragment thereof, wherein the first step-up dose is administered once, and the second step-up dose is administered once.

In some embodiments, the first step-up dose of the isolated anti-hK2/anti-CD3 bispecific antibody or bispecific fragment thereof is administered, via IV infusion, 3 days to 8 days prior to the administration of the second step-up dose.

In a particular embodiment, the first step-up dose of the isolated anti-hK2/anti-CD3 bispecific antibody or bispecific fragment thereof is administered, via IV infusion, 3 days prior to the administration of the second step-up dose.

In some embodiments, the method comprises administering to the subject a first step-up dose of between about 2 mg to about 5 mg per administration (for example a flat dose of between about 2 mg to about 5 mg per administration) and the first step-up dose is administered, via IV infusion, 3 days prior to the administration of the second step-up dose. In a particular embodiment, the first step-up dose is about 3.5 mg per administration (for example a flat dose of about 3.5 mg per administration) and the first step-up dose is administered, via IV infusion, 3 days prior to the administration of the second step-up dose.

In some embodiments, the method comprises administering to the subject a first step-up dose of between about 5 mg to about 15 mg per administration (for example a flat dose of between about 5 mg to about 15 mg per administration) and the first step-up dose is administered, via IV infusion, 3 days prior to the administration of the second step-up dose. In a particular embodiment, the first step-up dose is about 10 mg per administration (for example a flat dose of about 10 mg per administration) and the first step-up dose is administered, via IV infusion, 3 days prior to the administration of the second step-up dose.

In a particular embodiment, the first step-up dose of the isolated anti-hK2/anti-CD3 bispecific antibody or bispecific fragment thereof is administered, via IV infusion, 7 days prior to the administration of the second step-up dose.

In some embodiments, the method comprises administering to the subject a first step-up dose of between about 2 mg to about 5 mg per administration (for example a flat dose of between about 2 mg to about 5 mg per administration) and the first step-up dose is administered, via IV infusion, 7 days prior to the administration of the second step-up dose. In a particular embodiment, the first step-up dose is about 3.5 mg per administration (for example a flat dose of about 3.5 mg per administration) and the first step-up dose is administered, via IV infusion, 7 days prior to the administration of the second step-up dose.

In some embodiments, the method comprises administering to the subject a first step-up dose of between about 5 mg to about 15 mg per administration (for example a flat dose of between about 5 mg to about 15 mg per administration) and the first step-up dose is administered, via IV infusion, 7 days prior to the administration of the second step-up dose. In a particular embodiment, the first step-up dose is about 10 mg per administration (for example a flat dose of about 10 mg per administration) and the first step-up dose is administered, via IV infusion, 7 days prior to the administration of the second step-up dose.

In some embodiments, the second step-up dose of the isolated anti-hK2/anti-CD3 bispecific antibody or bispecific fragment thereof is administered, via IV infusion, 3 days to 8 days prior to the administration of the treatment dose. In certain embodiments, the second step-up dose is administered, via IV infusion, 3 days prior to the administration of the treatment dose.

In a particular embodiment, the second step-up dose of the isolated anti-hK2/anti-CD3 bispecific antibody or bispecific fragment thereof is administered, via IV infusion, 3 days prior to the administration of the treatment dose.

In some embodiments, the method comprises administering to the subject a second step-up dose of between about 5 mg to about 15 mg per administration (for example a flat dose of between about 5 mg to about 15 mg per administration) and the second step-up dose is administered, via IV infusion, 3 days prior to the administration of the treatment dose. In a particular embodiment, the second step-up dose is about 10 mg per administration (for example a flat dose of about 10 mg per administration) and the second step-up dose is administered, via IV infusion, 3 days prior to the administration of the treatment dose.

In some embodiments, the method comprises administering to the subject a second step-up dose of between about 15 mg to about 25 mg per administration (for example a flat dose of between about 15 mg to about 25 mg per administration) and the second step-up dose is administered, via IV infusion, 3 days prior to the administration of the treatment dose. In a particular embodiment, the second step-up dose is about 18 mg per administration (for example a flat dose of about 18 mg per administration) and the second step-up dose is administered, via IV infusion, 3 days prior to the administration of the treatment dose.

In some embodiments, the method comprises administering to the subject a second step-up dose of between about 200 mg to about 400 mg per administration (for example a flat dose of between about 200 mg to about 400 mg per administration) and the second step-up dose is administered, via IV infusion, 3 days prior to the administration of the treatment dose. In a particular embodiment, the second step-up dose is about 300 mg per administration (for example a flat dose of about 300 mg per administration) and the second step-up dose is administered, via IV infusion, 3 days prior to the administration of the treatment dose.

In a particular embodiment, the second step-up dose of the isolated anti-hK2/anti-CD3 bispecific antibody or bispecific fragment thereof is administered, via IV infusion, 7 days prior to the administration of the treatment dose.

In some embodiments, the method comprises administering to the subject a second step-up dose of between about 5 mg to about 15 mg per administration (for example a flat dose of between about 5 mg to about 15 mg per administration) and the second step-up dose is administered, via IV infusion, 7 days prior to the administration of the treatment dose. In a particular embodiment, the second step-up dose is about 10 mg per administration (for example a flat dose of about 10 mg per administration) and the second step-up dose is administered, via IV infusion, 7 days prior to the administration of the treatment dose.

In some embodiments, the method comprises administering to the subject a second step-up dose of between about 15 mg to about 25 mg per administration (for example a flat dose of between about 15 mg to about 25 mg per administration) and the second step-up dose is administered, via IV infusion, 7 days prior to the administration of the treatment dose. In a particular embodiment, the second step-up dose is about 18 mg per administration (for example a flat dose of about 18 mg per administration) and the second step-up dose is administered, via IV infusion, 7 days prior to the administration of the treatment dose.

In some embodiments, the method comprises administering to the subject a second step-up dose of between about 200 mg to about 400 mg per administration (for example a flat dose of between about 200 mg to about 400 mg per administration) and the second step-up dose is administered, via IV infusion, 7 days prior to the administration of the treatment dose. In a particular embodiment, the second step-up dose is about 300 mg per administration (for example a flat dose of about 300 mg per administration) and the second step-up dose is administered, via IV infusion, 7 days prior to the administration of the treatment dose.

The inventors have found that administration by intravenous infusion may be advantageous in reducing the likelihood of adverse events following administration of bispecific antibodies. The step-up doses may therefore also be administered by intravenous infusion. Accordingly, in some embodiments, the first step-up dose of the isolated anti-hK2/anti-CD3 bispecific antibody or bispecific fragment thereof is administered via intravenous (IV) infusion. In some embodiments, the second step-up dose of the isolated anti-hK2/anti-CD3 bispecific antibody or bispecific fragment thereof is administered via IV infusion. In some embodiments, the first step-up dose and the second step-up dose of the isolated anti-hK2/anti-CD3 bispecific antibody or bispecific fragment thereof is administered via intravenous (IV) infusion.

In some embodiments the method comprises administering to the subject a first step-up dose of the isolated anti-hK2/anti-CD3 bispecific antibody or bispecific fragment thereof administered by intravenous (IV) infusion. In some embodiments, the method comprises administering to the subject a second step-up dose of the isolated anti-hK2/anti-CD3 bispecific antibody or bispecific fragment thereof administered by intravenous (IV) infusion. In some embodiments, the method comprises administering to the subject a first step-up dose of the isolated anti-hK2/anti-CD3 bispecific antibody or bispecific fragment thereof administered by intravenous (IV) infusion and a second step-up dose of the isolated anti-hK2/anti-CD3 bispecific antibody or bispecific fragment thereof administered by intravenous (IV) infusion

An intravenous infusion of the isolated anti-hK2/anti-CD3 bispecific antibody or bispecific fragment thereof may be administered over several minutes to several hours. The inventors have found that administration of the first step-up dose of an isolated anti-hK2/anti-CD3 bispecific antibody or bispecific fragment thereof over a period of one hour followed by administration of subsequent infusions of the treatment dose over a period of 30 minutes are suitable timings for administration. Accordingly, in some embodiments, the first step-up dose is administered by intravenous (IV) infusion over a period of at least about one hour. In certain such embodiments, the first step-up dose is administered by intravenous (IV) infusion over a period of about one hour.

In some embodiments, the second step-up dose is administered by intravenous (IV) infusion over a period of at least about 30 minutes. In certain such embodiments, the second step-up dose is administered by intravenous (IV) infusion over a period of about 30 minutes.

In some embodiments, the treatment dose is administered by intravenous (IV) infusion over a period of at least about 30 minutes. In certain such embodiments, the treatment dose is administered by intravenous (IV) infusion over a period of about 30 minutes.

In some embodiments, the first step-up dose is administered by intravenous infusion over a period of at least one hour (for example over a period of about one hour); the second step-up dose is administered by intravenous infusion over a period of at least 30 minutes (for example over a period of about 30 minutes); and any subsequent treatment dose is administered by intravenous infusion over a period of at least 30 minutes (for example over a period of about 30 minutes).

### Dosing Regimens

The inventors have found that certain dosing regimens may be particularly suitable for the treatment of prostate cancer, as described elsewhere herein.

Accordingly, in some embodiments, the method of treating prostate cancer in a human subject in need thereof comprises administering a therapeutically effective amount of an of an isolated anti-hK2/anti-CD3 bispecific antibody or a bispecific fragment thereof to the subject at a treatment dose by intravenous (IV) infusion, wherein the method comprises administering to the subject: i) a step-up dose of the isolated anti-hK2/anti-CD3 bispecific antibody or bispecific fragment thereof; and ii) the treatment dose of the isolated anti-hK2/anti-CD3 bispecific antibody or bispecific fragment thereof, wherein the step-up dose is administered once, and wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

In certain embodiments, the step-up dose is administered prior to the treatment dose, the step-up dose is about 10 mg (for example a flat dose of about 10 mg) and the treatment dose is about 300 mg (for example a flat dose of about 300 mg).

In certain embodiments, the step-up dose is administered 7 days prior to the treatment dose.

In a particular embodiment, the method comprises administering to the subject a step-up dose of the isolated anti-hK2/anti-CD3 bispecific antibody or bispecific fragment thereof, wherein the step-up dose is administered 7 days prior to the treatment dose, wherein the step-up dose is about 10 mg (for example a flat dose of about 10 mg) and the treatment dose is about 300 mg per administration (for example a flat dose of about 300 mg).

In certain embodiments, the treatment dose is administered once every six weeks.

In a particular embodiment, the method comprises administering to the subject a step-up dose of the isolated anti-hK2/anti-CD3 bispecific antibody or bispecific fragment thereof, wherein the step-up dose is administered prior to the treatment dose, wherein the step-up dose is about 10 mg (for example a flat dose of about 10 mg) and the treatment dose is about 300 mg (for example a flat dose of about 300 mg) and the treatment dose is administered once every six weeks.

In a particular embodiment, the method comprises administering to the subject a step-up dose of the isolated anti-hK2/anti-CD3 bispecific antibody or bispecific fragment thereof, wherein the step-up dose is administered 7 days prior to the treatment dose, wherein the step-up dose is 10 mg (for example a flat dose of about 10 mg) and the treatment dose is 300 mg (for example a flat dose of about 300 mg) and the treatment dose is administered once every six weeks.

In certain embodiments, the treatment dose is administered once every three weeks.

In a particular embodiment, the method comprises administering to the subject a step-up dose of the isolated anti-hK2/anti-CD3 bispecific antibody or bispecific fragment thereof, wherein the step-up dose is administered prior to the treatment dose, wherein the step-up dose is about 10 mg (for example a flat dose of about 10 mg) and the treatment dose is about 300 mg (for example a flat dose of about 300 mg) and the treatment dose is administered once every three weeks.

In a particular embodiment, the method comprises administering to the subject a step-up dose of the isolated anti-hK2/anti-CD3 bispecific antibody or bispecific fragment thereof, wherein the step-up dose is administered 7 days prior to the treatment dose, wherein the step-up dose is 10 mg (for example a flat dose of about 10 mg) and the treatment dose is 300 mg (for example a flat dose of about 300 mg) and the treatment dose is administered once every three weeks.

In some embodiments, the method of treating prostate cancer in a human subject in need thereof comprises administering a therapeutically effective amount of an of an isolated anti-hK2/anti-CD3 bispecific antibody or a bispecific fragment thereof to the subject at a treatment dose by intravenous (IV) infusion, wherein the method comprises administering to the subject: i) a step-up dose of the isolated anti-hK2/anti-CD3 bispecific antibody or bispecific fragment thereof; and ii) the treatment dose of the isolated anti-hK2/anti-CD3 bispecific antibody or bispecific fragment thereof, wherein the step-up dose is administered once, and wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

In certain embodiments, the step-up dose is administered prior to the treatment dose, the step-up dose is about 10 mg (for example a flat dose of about 10 mg) and the treatment dose is about 600 mg (for example a flat dose of about 600 mg).

In certain embodiments, the step-up dose is administered 7 days prior to the treatment dose.

In a particular embodiment, the method comprises administering to the subject a step-up dose of the isolated anti-hK2/anti-CD3 bispecific antibody or bispecific fragment thereof, wherein the step-up dose is administered 7 days prior to the treatment dose, wherein the step-up dose is about 10 mg (for example a flat dose of about 10 mg) and the treatment dose is about 600 mg (for example a flat dose of about 600 mg).

In certain embodiments, the treatment dose is administered once every six weeks.

In a particular embodiment, the method comprises administering to the subject a step-up dose of the isolated anti-hK2/anti-CD3 bispecific antibody or bispecific fragment thereof, wherein the step-up dose is administered prior to the treatment dose, wherein the step-up dose is about 10 mg (for example a flat dose of about 10 mg) and the treatment dose is about 600 mg (for example a flat dose of about 600 mg) and the treatment dose is administered once every six weeks.

In a particular embodiment, the method comprises administering to the subject a step-up dose of the isolated anti-hK2/anti-CD3 bispecific antibody or bispecific fragment thereof, wherein the step-up dose is administered 7 days prior to the treatment dose, wherein the step-up dose is 10 mg (for example a flat dose of about 10 mg) and the treatment dose is 600 mg (for example a flat dose of about 600 mg) and the treatment dose is administered once every six weeks.

In certain embodiments, the treatment dose is administered once every three weeks.

In a particular embodiment, the method comprises administering to the subject a step-up dose of the isolated anti-hK2/anti-CD3 bispecific antibody or bispecific fragment thereof, wherein the step-up dose is administered prior to the treatment dose, wherein the step-up dose is about 10 mg (for example a flat dose of about 10 mg) and the treatment dose is about 600 mg (for example a flat dose of about 600 mg) and the treatment dose is administered once every three weeks.

In a particular embodiment, the method comprises administering to the subject a step-up dose of the isolated anti-hK2/anti-CD3 bispecific antibody or bispecific fragment thereof, wherein the step-up dose is administered 7 days prior to the treatment dose, wherein the step-up dose is 10 mg (for example a flat dose of about 10 mg) and the treatment dose is 600 mg (for example a flat dose of about 600 mg) and the treatment dose is administered once every three weeks.

In some embodiments, the method of treating prostate cancer in a human subject in need thereof comprises administering a therapeutically effective amount of an of an isolated anti-hK2/anti-CD3 bispecific antibody or a bispecific fragment thereof to the subject at a treatment dose by intravenous (IV) infusion, wherein the method comprises administering to the subject: i) a first step-up dose of the isolated anti-hK2/anti-CD3 bispecific antibody or bispecific fragment thereof; ii) a second step-up dose of the isolated anti-hK2/anti-CD3 bispecific antibody or bispecific fragment thereof; and iii) the treatment dose of the isolated anti-hK2/anti-CD3 bispecific antibody or bispecific fragment thereof, wherein the first step-up dose and the second step-up doses are administered once, and wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

In certain embodiments, the first step-up dose is administered prior to the second step-up dose, and the second step-up dose is administered prior to the treatment dose, wherein the first step-up dose is about 3.5 mg (for example a flat dose of about 3.5 mg), the second step-up dose is 18 mg (for example a flat dose of about 18 mg), and the treatment dose is 300 mg (for example a flat dose of about 300 mg).

In certain embodiments, the first step-up dose is administered 7 days prior to the second step-up dose.

In certain embodiments, the second step-up dose is administered 7 days prior to the treatment dose.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), and the treatment dose is about 300 mg per administration (for example a flat dose of about 300 mg).

In certain embodiments, the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered prior to the second step-up dose, the second step-up dose is administered prior to the treatment dose, the first step-up dose is about 3.5 mg (for example a flat dose of about 3.5 mg), the second step-up dose is about 18 mg (for example a flat dose of about 18 mg), the treatment dose is 300 mg per administration (for example a flat dose of about 300 mg per administration) and the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), the treatment dose is about 300 mg per administration (for example a flat dose of about 300 mg per administration) and the treatment dose is administered once every six weeks. In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), the treatment dose is about 300 mg per administration (for example a flat dose of about 300 mg per administration) and the treatment dose is administered once every 42 days+/−3 days.

In certain embodiments, the treatment dose is administered once every three weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered prior to the second step-up dose, the second step-up dose is administered prior to the treatment dose, the first step-up dose is about 3.5 mg (for example a flat dose of about 3.5 mg), the second step-up dose is about 18 mg (for example a flat dose of about 18 mg), the treatment dose is 300 mg per administration (for example a flat dose of about 300 mg per administration) and the treatment dose is administered once every three weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), the treatment dose is about 300 mg per administration (for example a flat dose of about 300 mg per administration) and the treatment dose is administered once every three weeks.

In some embodiments, the method of treating prostate cancer in a human subject in need thereof comprises administering a therapeutically effective amount of an of an isolated anti-hK2/anti-CD3 bispecific antibody or a bispecific fragment thereof to the subject at a treatment dose by intravenous (IV) infusion, wherein the method comprises administering to the subject: i) a first step-up dose of the isolated anti-hK2/anti-CD3 bispecific antibody or bispecific fragment thereof; ii) a second step-up dose of the isolated anti-hK2/anti-CD3 bispecific antibody or bispecific fragment thereof; and iii) the treatment dose of the isolated anti-hK2/anti-CD3 bispecific antibody or bispecific fragment thereof, wherein the first step-up dose and the second step-up dose are administered once, and wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

In certain embodiments, the first step-up dose is administered prior to the second step-up dose, the second step-up dose is administered prior to the treatment dose, the first step-up dose is about 3.5 mg (for example a flat dose of about 3.5 mg), the second step-up dose is 18 mg (for example a flat dose of about 18 mg), and the treatment dose is 600 mg (for example a flat dose of about 600 mg).

In certain embodiments, the first step-up dose is administered 7 days prior to the second step-up dose.

In certain embodiments, the second step-up dose is administered 7 days prior to the treatment dose.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), and the treatment dose is about 600 mg per administration (for example a flat dose of about 600 mg).

In certain embodiments, the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered prior to the second step-up dose, the second step-up dose is administered prior to the treatment dose, the first step-up dose is about 3.5 mg (for example a flat dose of about 3.5 mg), the second step-up dose is about 18 mg (for example a flat dose of about 18 mg), the treatment dose is 600 mg per administration (for example a flat dose of about 600 mg per administration) and the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), the treatment dose is about 600 mg per administration (for example a flat dose of about 600 mg per administration) and the treatment dose is administered once every six weeks.

In certain embodiments, the treatment dose is administered once every three weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered prior to the second step-up dose, the second step-up dose is administered prior to the treatment dose, the first step-up dose is about 3.5 mg (for example a flat dose of about 3.5 mg), the second step-up dose is about 18 mg (for example a flat dose of about 18 mg), the treatment dose is 600 mg per administration (for example a flat dose of about 600 mg per administration) and the treatment dose is administered once every three weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 3.5 mg (for example a flat dose of about 3.5 mg), the second step-up dose is about 18 mg (for example a flat dose of about 18 mg), the treatment dose is about 600 mg per administration (for example a flat dose of about 600 mg per administration) and the treatment dose is administered once every three weeks.

In some embodiments, the method of treating prostate cancer in a human subject in need thereof comprises administering a therapeutically effective amount of an of an isolated anti-hK2/anti-CD3 bispecific antibody or a bispecific fragment thereof to the subject at a treatment dose by intravenous (IV) infusion, wherein the method comprises administering to the subject: i) a first step-up dose of the isolated anti-hK2/anti-CD3 bispecific antibody or bispecific fragment thereof; ii) a second step-up dose of the isolated anti-hK2/anti-CD3 bispecific antibody or bispecific fragment thereof; and iii) the treatment dose of the isolated anti-hK2/anti-CD3 bispecific antibody or bispecific fragment thereof, wherein the first step-up dose and the second step-up dose are administered once, and wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

In certain embodiments, the first step-up dose is administered prior to the second step-up dose, the second step-up dose is administered prior to the treatment dose, the first step-up dose is about 10 mg (for example a flat dose of about 10 mg), the second step-up dose is 300 mg (for example a flat dose of about 300 mg) and the treatment dose is 900 mg (for example a flat dose of about 900 mg).

In certain embodiments, the first step-up dose is administered 7 days prior to the second step-up dose.

In certain embodiments, the second step-up dose is administered 7 days prior to the treatment dose.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 10 mg (for example a flat dose of about 10 mg), the second step-up dose is about 300 mg (for example a flat dose of about 300 mg) and the treatment dose is about 900 mg per administration (for example a flat dose of about 900 mg).

In certain embodiments, the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered prior to the second step-up dose, the second step-up dose is administered prior to the treatment dose, the first step-up dose is about 10 mg (for example a flat dose of about 10 mg), the second step-up dose is about 300 mg (for example a flat dose of about 300 mg), the treatment dose is 900 mg per administration (for example a flat dose of about 900 mg per administration) and the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 10 mg (for example a flat dose of about 10 mg), the second step-up dose is about 300 mg (for example a flat dose of about 300 mg), the treatment dose is about 900 mg per administration (for example a flat dose of about 900 mg per administration) and the treatment dose is administered once every six weeks.

In some embodiments, the method of treating prostate cancer in a human subject in need thereof comprises administering a therapeutically effective amount of an of an isolated anti-hK2/anti-CD3 bispecific antibody or a bispecific fragment thereof to the subject at a treatment dose by intravenous (IV) infusion, wherein the method comprises administering to the subject: i) a first step-up dose of the isolated anti-hK2/anti-CD3 bispecific antibody or bispecific fragment thereof; ii) a second step-up dose of the isolated anti-hK2/anti-CD3 bispecific antibody or bispecific fragment thereof; and iii) the treatment dose of the isolated anti-hK2/anti-CD3 bispecific antibody or bispecific fragment thereof, wherein the first step-up dose and the second step-up dose are administered once, and wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

In certain embodiments, the first step-up dose is administered prior to the second step-up dose and the second step-up dose is administered prior to the treatment dose, wherein the first step-up dose is about 10 mg (for example a flat dose of about 10 mg), the second step-up dose is 300 mg (for example a flat dose of about 300 mg), and the treatment dose is 600 mg (for example a flat dose of about 600 mg).

In certain embodiments, the first step-up dose is administered 7 days prior to the second step-up dose.

In certain embodiments, the second step-up dose is administered 7 days prior to the treatment dose.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, wherein the first step-up dose is about 10 mg (for example a flat dose of about 10 mg), the second step-up dose is about 300 mg (for example a flat dose of about 300 mg), the treatment dose is about 600 mg per administration (for example a flat dose of about 600 mg).

In certain embodiments, the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered prior to the second step-up dose, the second step-up dose is administered prior to the treatment dose, the first step-up dose is about 10 mg (for example a flat dose of about 10 mg), the second step-up dose is about 300 mg (for example a flat dose of about 300 mg), the treatment dose is 600 mg per administration (for example a flat dose of about 600 mg per administration) and the treatment dose is administered once every six weeks.

In a particular embodiment, the method comprises administering to the subject a first step-up dose and a second step-up dose of the isolated anti-hK2/anti-CD3 bispecific antibody or bispecific fragment thereof, wherein the first step-up dose is administered 7 days prior to the second step-up dose, the second step-up dose is administered 7 days prior to the treatment dose, the first step-up dose is about 10 mg (for example a flat dose of about 10 mg), the second step-up dose is about 300 mg (for example a flat dose of about 300 mg), the treatment dose is about 600 mg per administration (for example a flat dose of about 600 mg per administration) and the treatment dose is administered once every six weeks.

In any of the dosing regimen embodiments described in the preceding paragraphs, the isolated anti-hK2/anti-CD3 antibody or bispecific fragment thereof may comprise a HC1 of SEQ ID NO: 9, a LC1 of SEQ ID NO: 10 and a HC2 of SEQ ID NO: 19.

### hk2 x CD3 Bispecific Antibodies

The methods provided herein comprise administering bispecific antibodies that bind to hk2 and CD3 (anti-hk2 x anti-CD3 bispecific antibodies) or bispecific binding fragment thereof.

The hk2 x CD3-bispecific antibody or bispecific binding fragment thereof comprises a first antigen binding domain that binds specifically to hK2, and a second antigen binding domain that binds specifically to CD3, wherein (a) the first domain that binds hK2 comprises a first heavy chain variable region (VH1) of SEQ ID NO: 7 and a first light chain variable region (VL1) of SEQ ID NO: 8; and (b) the second domain that binds CD3 comprises a second heavy chain variable domain (VH2) of SEQ ID NO: 17 and a second light chain variable domain (VL2) of SEQ ID NO: 18.

In some embodiments, the first domain that binds hK2 comprises a scFv, a (scFv)2, a Fv, a Fab, or a F(ab′)2. In a particular embodiment, the first domain that binds hK2 comprises a Fab.

In some embodiments, the second domain that binds CD36 comprises a scFv, a (scFv)2, a Fv, a Fab, or a F(ab′)2. In a particular embodiment, the second domain that binds CD36 comprises a scFv, for example an scFv comprising the amino acid sequence of SEQ ID NO: 59.

In some embodiments, the first domain that binds hK2 comprises a scFv, a (scFv)2, a Fv, a Fab, or a F(ab′)2 and the second domain that binds CD3ε comprises a scFv, a (scFv)2, a Fv, a Fab, or a F(ab′)2.

In a particular embodiment, the first domain that binds hK2 comprises an antigen-binding fragment (Fab), and the second domain that binds CD36 comprises a single-chain variable fragment (scFv), for example an scFv comprising the amino acid sequence of SEQ ID NO: 59.

In various embodiments, the scFvs used in bispecific antibodies or bispecific binding fragments described herein comprise, from the N- to C-terminus, a VH, a linker and a VL (VH-L-VL) or the VL, the linker and the VH (VL-L-VH). In some embodiments, the scFv comprises, from the N- to C-terminus, the VL, the linker and the VH (VL-L-VH).

In some embodiments, the linker L comprises about 5-50 amino acids, about 5-40 amino acids, about 10-30 amino acids, or about 10-20 amino acids.

In some embodiments, the linker comprises the amino acid sequence of SEQ ID NO: 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53.

In a particular embodiment, the linker comprises the amino acid sequence of SEQ ID NO: 20.

In various embodiments, the first domain that binds hK2 is conjugated to an immunoglobulin (Ig) heavy chain constant region, or a fragment of the first Ig heavy chain constant region. In various embodiments, the second domain that binds CD3ε is conjugated to an immunoglobulin (Ig) heavy chain constant region, or a fragment of the second Ig heavy chain constant region. In some embodiments, the first domain that binds hK2 is conjugated to a first immunoglobulin (Ig) heavy chain constant region, or a fragment of the first Ig heavy chain constant region, and the second domain that binds CD3 is conjugated to a second immunoglobulin (Ig) heavy chain constant region, or a fragment of the second Ig heavy chain constant region.

In some embodiments, the Ig heavy chain constant regions is an IgG1 isotype. In some embodiments, the first Ig heavy chain constant region, or the fragment of the first Ig heavy chain constant region, is an IgG1 isotype. In some embodiments, the second Ig heavy chain constant region, or the fragment of the second Ig heavy chain constant region, is an IgG1 isotype. In some embodiments, the first Ig heavy chain constant region, or the fragment of the first Ig heavy chain constant region, is an IgG1 isotype and the second Ig heavy chain constant region, or the fragment of the second Ig heavy chain constant region, is an IgG1 isotype.

In some embodiments, the bispecific antibodies of the invention include antibodies having a full length antibody structure. The full length antibody may be lacking the C-terminal lysine (K) in either one or both heavy chains.

The “knob-in-hole” strategy (see, e.g., PCT Inti. Publ. No. WO 2006/028936) may be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody binding a first antigen and an amino acid with a large side chain (knob) is introduced into a heavy chain of an antibody binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S/L368A/Y407V, wherein numbering of the amino acid mutations is according to the EU index.

In some embodiments of the bispecific antibody or bispecific binding fragment described herein, the first Ig constant region or the fragment of the first Ig constant region comprises the following mutations T350V/T366L/K392L/T394W. In some embodiments of the bispecific antibody or bispecific binding fragment described herein, the second Ig constant region or the fragment of the second Ig constant region comprises the following mutations T350V/L351Y/F405A/Y407V. In a particular embodiment of the bispecific antibody or bispecific binding fragment described herein, the first Ig constant region or the fragment of the first Ig constant region comprises the following mutations T350V/T366L/K392L/T394W and/or the second Ig constant region or the fragment of the second Ig constant region comprises the following mutations T350V/L351Y/F405A/Y407V.

In some embodiments wherein the antibody has an IgG1 isotype, the antibody contains L234A, L235A, D265S and/or K409R substitution(s) in its Fc region, wherein numbering of the amino acid mutations is according to the EU index. The antibodies described herein may include these modifications.

In some embodiments, the Fc domains of a bispecific antibody described herein each comprise one or more mutations selected from L234A, L235A, and D265S. In some embodiments, the Fc domains of HC1 and HC2 each comprise mutations L234A, L235A, and D265S.

In some embodiments, the bispecific antibodies or antigen-binding fragments are IgG, or derivatives thereof.

In some embodiments, the first Ig heavy chain constant region comprises the Fc silencing mutation (L234A/L235A/D265S) and the T350V/T366L/K392L/T394W mutations designed to promote selective heterodimerization, wherein numbering of the amino acid mutations is according to the EU index. In some embodiments, the antigen binding domain that binds hk2 is conjugated to an Ig heavy chain constant region (e.g. CH1-hinge-CH2-CH3) comprising an amino acid sequence which is at least 80% (e.g. at least 85%, at least 90%, at least 95% or at least 99%) identical to the amino acid sequence of SEQ ID NO: 54. In a particular embodiment, the antigen binding domain that binds hk2 is conjugated to an Ig heavy chain constant region or a fragment of the Ig heavy chain constant region comprising the amino acid sequence of SEQ ID NO: 54.

In some embodiments, the second Ig heavy chain constant region comprises the Fc silencing mutation (L234A/L235A/D265S) and the T350V/L351Y/F405A/Y407V mutations designed to promote selective heterodimerization, wherein numbering of the amino acid mutations is according to the EU index. In some embodiments, the antigen binding domain that binds CD3 is conjugated to an Ig heavy chain constant region (e.g. hinge-CH2-CH3) comprising an amino acid sequence which is at least 80% (e.g. at least 85%, at least 90%, at least 95% or at least 99%) identical to the amino acid sequence of SEQ ID NO: 55. In a particular embodiment, the antigen binding domain that binds CD3 is conjugated to an Ig heavy chain constant region or a fragment of the Ig heavy chain constant region comprising the amino acid sequence of SEQ ID NO: 55.

In some embodiments, the antigen binding domain that binds hk2 is conjugated to an Ig light chain constant region, or the fragment of the first Ig light chain constant region. In some embodiments, the antigen binding domain that binds hk2 is conjugated to an Ig light chain constant region, or the fragment of the first Ig light chain constant region comprising an amino acid sequence which is at least 80% (e.g. at least 85%, at least 90%, at least 95% or at least 99%) identical to SEQ ID NO: 56. In a particular embodiment, the antigen binding domain that binds hk2 is conjugated to an Ig light chain constant region, or the fragment of the first Ig light chain constant region comprising the amino acid sequence of SEQ ID NO: 56.

In a particular embodiment, the isolated bispecific antibody, or a bispecific binding fragment thereof disclosed herein comprises an antigen binding domain that binds hk2 which is conjugated to an IgG1 heavy chain constant region comprising the amino acid sequence of SEQ ID NO: 54 and an antigen binding domain that binds hk2 which is conjugated to an IgG1 light chain constant region comprising an amino acid sequence which is identical to SEQ ID NO: 56.

In some embodiments, the first Ig heavy chain constant region or a fragment of the Ig heavy chain constant region is conjugated to a hk2 binding domain that comprises a Fab. In some embodiments, the antigen binding domain that binds hk2 is a Fab comprising a VH with an amino acid sequence comprising SEQ ID NO: 7 and a VL with an amino acid sequence comprising SEQ ID NO: 8 that is conjugated to an Ig heavy chain constant region or a fragment of the Ig heavy chain constant region comprising the amino acid sequence of SEQ ID NO: 54.

In some embodiments, the second Ig heavy chain constant region or a fragment of the Ig heavy chain constant region is conjugated to a CD3 binding domain that comprises a scFv, for example an scFv comprising the amino acid sequence of SEQ ID NO: 59, that is conjugated to an Ig heavy chain constant region or a fragment of the Ig heavy chain constant region comprising the amino acid sequence of SEQ ID NO: 55.

In a particular embodiment, the isolated bispecific antibody, or a bispecific binding fragment thereof disclosed herein comprises (i) a first domain that binds hk2 comprising a Fab comprising a VH with an amino acid sequence comprising SEQ ID NO: 7 and a VL with an amino acid sequence comprising SEQ ID NO: 8 that is conjugated to an Ig heavy chain constant region or a fragment of the Ig heavy chain constant region comprising the amino acid sequence of SEQ ID NO: 54 and (ii) a second domain that binds CD3 comprising an scFv comprising the amino acid sequence of SEQ ID NO: 59 that is conjugated to an Ig heavy chain constant region or a fragment of the Ig heavy chain constant region comprising the amino acid sequence of SEQ ID NO: 55.

In a particular embodiment, the isolated bispecific antibody, or a bispecific binding fragment thereof disclosed herein comprises a first antigen binding domain that binds hk2 and (i) a first Ig heavy chain constant region or fragment of a first Ig heavy chain constant region comprising an amino acid sequence which is identical to SEQ ID NO: 54; and (ii) a first Ig light chain constant region or fragment of a first Ig light chain constant region comprising an amino acid sequence which is identical to SEQ ID NO: 56, and wherein the isolated bispecific antibody, or a bispecific binding fragment thereof further comprises a second antigen binding domain that binds CD3 and a second Ig constant region or a fragment of a second Ig constant region comprising an amino acid sequence which is identical to SEQ ID NO: 55.

In a particular embodiment, the isolated bispecific antibody, or a bispecific binding fragment thereof disclosed herein comprises a first antigen binding domain that binds hk2 comprising a Fab comprising a VH with an amino acid sequence comprising SEQ ID NO: 7 and a VL with an amino acid sequence comprising SEQ ID NO: 8 that is conjugated to (i) a first Ig heavy chain constant region or fragment of a first Ig heavy chain constant region comprising an amino acid sequence which is identical to SEQ ID NO: 54; and (ii) a first Ig light chain constant region or fragment of a first Ig light chain constant region comprising an amino acid sequence which is identical to SEQ ID NO: 56, and wherein the isolated bispecific antibody, or a bispecific binding fragment thereof further comprises a second antigen binding domain that binds CD3 comprising an scFv comprising the amino acid sequence of SEQ ID NO: 59 that is conjugated to a second Ig constant region or a fragment of a second Ig constant region comprising an amino acid sequence which is identical to SEQ ID NO: 55.

In some embodiments, the hk2-binding arm of the bispecific antibody comprises a HC1 and a LC. The HC1 comprises constant heavy chain regions (CH1, CH2, and CH3) and the VH1. The LC comprises the VL1. The VH1 and VL1 combine to form the hk2 antigen binding domain.

In some embodiments, the CD3-binding arm of the bispecific antibody comprises a HC2. The HC2 comprises constant heavy chain regions (CH2 and CH3), and a single-chain variable fragment (scFv) attached at the N-terminus of the CH2 region, wherein the scFv comprises the CD3 antigen binding domain.

In one embodiment, the VH and VL of the Fab comprising the antigen binding domain that binds hk2 may be engineered into Fab-Fc HC1 (VH-CH1-hinge-CH2-CH3) and Fab-Fc LC (VL-CL) formats respectively. In certain such embodiments, the Fab-Fc HC1 comprises an amino acid sequence which is at least 80% (e.g. at least 85%, at least 90%, at least 95% or at least 99%) identical to SEQ ID NO: 57. In a particular embodiment, the Fab-Fc HC1 comprises an amino acid sequence which is identical to SEQ ID NO: 57.

In some embodiments, the Fab-Fc LC comprises an amino acid sequence which is at least 80% (e.g. at least 85%, at least 90%, at least 95% or at least 99%) identical to SEQ ID NO: 10. In a particular embodiment, the Fab-Fc LC comprises an amino acid sequence which is identical to SEQ ID NO: 10.

In one embodiment, the VH and VL of the scFv comprising the antigen binding domain that binds CD3 may be engineered into scFv-Fc HC2 (scFv-hinge-CH2-CH3). In certain such embodiments, the scFv-Fc HC2 comprises an amino acid sequence which is at least 80% (e.g. at least 85%, at least 90%, at least 95% or at least 99%) identical to SEQ ID NO: 58. In a particular embodiment, the Fab-Fc HC2 comprises an amino acid sequence which is identical to SEQ ID NO: 58.

In some embodiments, the Fab-Fc HC1 comprises a C-terminal lysine residue (e.g. K477). In some embodiments, the Fab-Fc HC1 comprises an amino acid sequence having a C-terminal lysine residue and a sequence which is at least 80% (e.g. at least 85%, at least 90%, at least 95% or at least 99%) identical to SEQ ID NO: 9. In a particular embodiment, the Fab-Fc HC1 comprises an amino acid sequence of SEQ ID NO: 9.

In some embodiments, the scFv-Fc HC2 comprises a C-terminal lysine residue (e.g. K477). In some embodiments, the scFv-Fc HC2 comprises an amino acid sequence having a C-terminal lysine residue and a sequence which is at least 80% (e.g. at least 85%, at least 90%, at least 95% or at least 99%) identical to SEQ ID NO: 19. In a particular embodiment, the scFv-Fc HC2 comprises an amino acid sequence of SEQ ID NO: 19.

In one embodiment, the bispecific antibody or bispecific fragment thereof comprises (i) a first antigen binding domain that binds hk2, wherein the first antigen binding domain comprises an HC1 which is at least 80% (for example at least 95%) identical to the HC1 of SEQ ID NO: 9, and LC1 that is at least 80% (for example at least 95%) identical to the LC1 of SEQ ID NO: 10; and (ii) a second antigen binding domain that binds CD3, wherein the second antigen binding domain comprises an HC2 which is at least 80% (for example at least 95%) identical to the HC2 of SEQ ID NO: 19.

In a particular embodiment, the isolated bispecific antibody, or a bispecific binding fragment thereof, comprises a HC1 of SEQ ID NO: 9, a LC1 of SEQ ID NO: 10 and a HC2 of SEQ ID NO: 19.

In one embodiment, the bispecific antibody or bispecific fragment thereof comprises (i) a first antigen binding domain that binds hk2, wherein the first antigen binding domain comprises an HC1 which is at least 80% (for example at least 95%) identical to the HC1 of SEQ ID NO: 57, and LC1 that is at least 80% (for example at least 95%) identical to the LC1 of SEQ ID NO: 10; and (ii) a second antigen binding domain that binds CD3, wherein the second antigen binding domain comprises an HC2 which is at least 80% (for example at least 95%) identical to the HC2 of SEQ ID NO: 58.

In a particular embodiment, the isolated bispecific antibody, or a bispecific binding fragment thereof, comprises a HC1 of SEQ ID NO: 57, a LC1 of SEQ ID NO: 10 and a HC2 of SEQ ID NO: 58.

In some embodiments, the hk2 x CD3 bispecific antibody or bispecific binding fragment thereof comprises any of the hk2 x CD3 bispecific antibodies described in WO2021/019389, the entire content of which is incorporated herein by reference.

The hk2 x CD3 bispecific antibodies or bispecific binding fragment thereof can be provided by making a molecule which comprises a first antigen-binding arm binding to CD3, and a second antigen-binding arm binding to hk2. Accordingly, the methods comprise administering bispecific molecules comprising two different antigen-binding arms which bind hk2 and CD3, respectively.

### Treatment of Prostate Cancer

The inventors have found that treatment with an isolated anti-hK2/anti-CD3 bispecific antibody or bispecific fragment thereof according to the methods disclosed herein may lead to safe and effective outcomes for subjects in need of treatment for prostate cancer. Certain subjects (e.g. subjects having certain types of prostate cancer, or certain prior therapies) may benefit from the methods disclosed herein.

In some embodiments, the prostate cancer is advanced prostate cancer.

In certain embodiments, the prostate cancer is advanced prostate cancer with a prostate adenocarcinoma that has small cell or neuroendocrine (NE) features. In certain embodiments, the prostate cancer is advanced prostate cancer with a prostate adenocarcinoma that does not comprise small cell carcinoma, carcinoid tumor, mixed NE carcinoma, or large cell NE carcinoma. In certain embodiments, the prostate cancer is advanced prostate cancer with a prostate adenocarcinoma prostate adenocarcinoma that has small cell or neuroendocrine (NE) features and that does not comprise small cell carcinoma, carcinoid tumor, mixed NE carcinoma, or large cell NE carcinoma. In some embodiments, the prostate cancer is metastatic prostate cancer. In a particular embodiment, the prostate cancer is metastatic castration-resistant prostate cancer (mCRPC).

In certain embodiments, the prostate cancer is mCRPC with a prostate adenocarcinoma that has small cell or neuroendocrine (NE) features. In certain embodiments, the prostate cancer is mCRPC with a prostate adenocarcinoma that does not comprise small cell carcinoma, carcinoid tumor, mixed NE carcinoma, or large cell NE carcinoma. In certain embodiments, the prostate cancer is mCRPC with a prostate adenocarcinoma prostate adenocarcinoma that has small cell or neuroendocrine (NE) features and that does not comprise small cell carcinoma, carcinoid tumor, mixed NE carcinoma, or large cell NE carcinoma. In some embodiments, the prostate cancer is histologically confirmed adenocarcinoma of the prostate, as defined by Prostate Cancer Working Group 3 (PCWG3) criteria (Scher et al. 2016 Journal of Clinical Oncology 34(12): 1402-1418).

In some embodiments, the prostate cancer is histologically confirmed adenocarcinoma of the prostate, as defined by Prostate Cancer Working Group 3 (PCWG3) criteria (Scher et al. 2016 Journal of Clinical Oncology 34(12): 1402-1418), wherein the prostate adenocarcinoma has small cell or neuroendocrine (NE) features.

In some embodiments, the prostate cancer is histologically confirmed adenocarcinoma of the prostate, as defined by Prostate Cancer Working Group 3 (PCWG3) criteria ((Scher et al. 2016 Journal of Clinical Oncology 34(12): 1402-1418)), wherein the prostate adenocarcinoma does not comprise small cell carcinoma, carcinoid tumor, mixed NE carcinoma, or large cell NE carcinoma.

In some embodiments, the prostate cancer is histologically confirmed adenocarcinoma of the prostate, as defined by Prostate Cancer Working Group 3 (PCWG3) criteria ((Scher et al. 2016 Journal of Clinical Oncology 34(12): 1402-1418)), wherein prostate adenocarcinoma has small cell or neuroendocrine (NE) features and does not comprise small cell carcinoma, carcinoid tumor, mixed NE carcinoma, or large cell NE carcinoma.

In some embodiments, the human subject is 18 years or older.

In some embodiments, the subject has received prior treatment for metastatic castration resistant prostate cancer (mCRPC) with at least one androgen receptor (AR)-targeted therapy (for example treatment with abiraterone acetate, apalutamide, enzalutamide, or darolutamide), or at least one chemotherapy (for example docetaxel).

In some embodiments, the prostate cancer is mCRPC that is metastatic to bone and/or lymph node, without evidence of metastasis to visceral organs. In certain embodiments, the subject has no known prior history of CNS involvement in prostate cancer. In certain embodiments the subject has a PSA level of at least 2 ng/ml prior to treatment. In certain embodiments the subject has received prior treatment for mCRPC with at least one androgen receptor (AR)-targeted therapy (for example treatment with abiraterone acetate, apalutamide, enzalutamide, or darolutamide). In certain embodiments, the subject has received no more than two prior taxane therapies. Throughout the application, the expression “taxane therapy” refers to a “taxane regimen” as understood by the person of ordinary skill in the art, for example exposure to 2 cycles of a taxane. In certain embodiments, the subject has not received prior cytotoxic chemotherapy (for example, cisplatin, carboplatin or etoposide).

In some embodiments, the subject has received prior treatment for mCRPC with at least one androgen receptor (AR)-targeted therapy (for example treatment with abiraterone acetate, apalutamide, enzalutamide, or darolutamide) and is unlikely to benefit from retreatment with another androgen receptor (AR)-targeted therapy. In some embodiments, the subject has received prior treatment for mCRPC with at least one taxane therapy. In some embodiments, the subject has received prior treatment for mCRPC with at least 2 prior taxane therapies. In some embodiments, the subject has received prior treatment for mCRPC with at least 2 prior taxane regimens, wherein a taxane regimen is defined as a minimum exposure of 2 cycles of a taxane. In some embodiments, the patient has received prior treatment for mCRPC with only 1 taxane regimen and is unsuitable to receive a second taxane regimen due to toxicity risk of prior intolerance. In some embodiments, the patient has received prior treatment for mCRPC with only 1 taxane regimen and has not received prior treatment with Cabazitaxel. In some embodiments, the patient has received prior treatment for mCRPC with at least one dose of PSMA-targeted lutetium radioligand therapy (for example, lutetium Lu-177 vipivotide tetraxetan). In some embodiments, the patient has received prior treatment for mCRPC with a polyadenosine diphosphate-ribose polymerase inhibitors (PARPi). In some embodiments, the serum Prostate-Specific Antigen (PSA) level of the subject measured once the prior treatment with the androgen receptor (AR)-targeted therapy, the taxane therapy, the PSMA-targeted lutetium radioligand therapy or the PARPi has been completed, is not decreased compared to the PSA level measured before the administration of the androgen receptor (AR)-targeted therapy, the taxane therapy, the PSMA-targeted lutetium radioligand therapy or the PARPi, respectively.

In a particular embodiment, the prostate cancer is mCRPC that is metastatic to bone and/or lymph node, without evidence of metastasis to visceral organs; the subject has no known prior history of CNS involvement in prostate cancer; the subject has a PSA level of at least 2 ng/ml prior to treatment; the subject has received prior treatment for mCRPC with at least one androgen receptor (AR)-targeted therapy (for example treatment with abiraterone acetate, apalutamide, enzalutamide, or darolutamide); the subject has received no more than two prior taxane therapies; and the subject has not received prior cytotoxic chemotherapy (for example, cisplatin, carboplatin or etoposide).

In some embodiments, the prostate cancer is mCRPC that is metastatic to bone and/or lymph node, without evidence of metastasis to visceral organs. In certain embodiments, the subject has no known prior history of CNS involvement in prostate cancer. In certain embodiments the subject has a PSA level of at least 2 ng/ml prior to treatment. In certain embodiments the subject has received prior treatment for mCRPC with at least one androgen receptor (AR)-targeted therapy (for example treatment with abiraterone acetate, apalutamide, enzalutamide, or darolutamide). In certain embodiments, the subject has not received prior cytotoxic chemotherapy (for example, cisplatin, carboplatin or etoposide). In certain embodiments, the subject has not previously received chemotherapy.

In a particular embodiment, the prostate cancer is mCRPC that is metastatic to bone and/or lymph node, without evidence of metastasis to visceral organs; the subject has no known prior history of CNS involvement in prostate cancer; the subject has a PSA level of at least 2 ng/ml prior to treatment; the subject has received prior treatment for mCRPC with at least one androgen receptor (AR)-targeted therapy (for example treatment with abiraterone acetate, apalutamide, enzalutamide, or darolutamide); the subject has not received prior cytotoxic chemotherapy (for example, cisplatin, carboplatin or etoposide); and the subject has not previously received any other chemotherapy.

In some embodiments, the subject has prostate cancer that is metastatic to visceral tissue. In certain embodiments, the subject has no known prior history of CNS involvement in prostate cancer. In certain embodiments, the subject has a PSA level of at least 10 ng/ml prior to treatment. In certain embodiments, the subject has received prior treatment with at least one androgen receptor (AR)-targeted therapy (for example treatment with abiraterone acetate, apalutamide, enzalutamide, or darolutamide). In certain embodiments, the subject has received no more than two prior taxane therapies. In certain embodiments, wherein the subject has not received prior cytotoxic chemotherapy (for example, cisplatin, carboplatin or etoposide).

In a particular embodiment, the subject has prostate cancer that is metastatic to visceral tissue; the subject has no known prior history of CNS involvement in prostate cancer; the subject has a PSA level of at least 10 ng/ml prior to treatment; the subject has received prior treatment with at least one androgen receptor (AR)-targeted therapy (for example treatment with abiraterone acetate, apalutamide, enzalutamide, or darolutamide); the subject has received no more than two prior taxane therapies; and the subject has not received prior cytotoxic chemotherapy (for example, cisplatin, carboplatin or etoposide).

In some embodiments, the subject has received orchiectomy or medical castration; or

has not undergone orchiectomy and is receiving ongoing androgen deprivation therapy with a gonadotropin releasing hormone (GnRH) analog (agonist or antagonist) prior to being administered with the isolated anti-hK2/anti-CD3 bispecific antibody or bispecific fragment thereof.

In a particular embodiment, the prostate cancer is mCRPC that is metastatic to bone and/or lymph node, without evidence of metastasis to visceral organs; the subject has no known prior history of CNS involvement in prostate cancer; the subject has a PSA level of at least 2 ng/ml prior to treatment; the subject has received prior treatment for mCRPC with at least one androgen receptor (AR)-targeted therapy (for example treatment with abiraterone acetate, apalutamide, enzalutamide, or darolutamide); the subject has received no more than two prior taxane therapies; the subject has not received prior cytotoxic chemotherapy (for example, cisplatin, carboplatin or etoposide); and the subject has received orchiectomy or medical castration; or has not undergone orchiectomy and is receiving ongoing androgen deprivation therapy with a gonadotropin releasing hormone (GnRH) analog (agonist or antagonist) prior to being administered with the isolated anti-hK2/anti-CD3 bispecific antibody or bispecific fragment thereof.

In a particular embodiment, the prostate cancer is mCRPC that is metastatic to bone and/or lymph node, without evidence of metastasis to visceral organs; the subject has no known prior history of CNS involvement in prostate cancer; the subject has a PSA level of at least 2 ng/ml prior to treatment; the subject has received prior treatment for mCRPC with at least one androgen receptor (AR)-targeted therapy (for example treatment with abiraterone acetate, apalutamide, enzalutamide, or darolutamide); the subject has not received prior cytotoxic chemotherapy (for example, cisplatin, carboplatin or etoposide); the subject has not previously received any other chemotherapy; and the subject has received orchiectomy or medical castration; or has not undergone orchiectomy and is receiving ongoing androgen deprivation therapy with a gonadotropin releasing hormone (GnRH) analog (agonist or antagonist) prior to being administered with the isolated anti-hK2/anti-CD3 bispecific antibody or bispecific fragment thereof.

In a particular embodiment embodiments, the subject has prostate cancer that is metastatic to visceral tissue; the subject has no known prior history of CNS involvement in prostate cancer; the subject has a PSA level of at least 10 ng/ml prior to treatment; the subject has received prior treatment with at least one androgen receptor (AR)-targeted therapy (for example treatment with abiraterone acetate, apalutamide, enzalutamide, or darolutamide); the subject has received no more than two prior taxane therapies; the subject has not received prior cytotoxic chemotherapy (for example, cisplatin, carboplatin or etoposide); and the subject has received orchiectomy or medical castration; or has not undergone orchiectomy and is receiving ongoing androgen deprivation therapy with a gonadotropin releasing hormone (GnRH) analog (agonist or antagonist) prior to being administered with the isolated anti-hK2/anti-CD3 bispecific antibody or bispecific fragment thereof.

In some embodiments, the subject has oligometastatic metastatic castration-sensitive prostate cancer (mCSPC). In certain embodiments, the subject has recurrent disease after definitive treatment to prostate, wherein the prostate cancer is metastatic to bone and/or lymph node, without evidence of metastasis to visceral organs. In certain embodiments, the subject has no more than five total lesions on imaging, for example up to five lesions PSMA-PET or no more than three lesions by conventional imaging. In certain embodiments, the subject has not received an ADT or ARPI within one year of administration of the isolated anti-hK2/anti-CD3 bispecific antibody or bispecific fragment thereof. In certain embodiments, wherein the subject has non-castrate levels of testosterone of greater than 150 ng/mL.

In some embodiments, the subject has oligometastatic metastatic castration-sensitive prostate cancer (mCSPC); the subject has recurrent disease after definitive treatment to prostate; the prostate cancer is metastatic to bone and/or lymph node, without evidence of metastasis to visceral organs; the subject has no more than five total lesions on imaging, for example up to five lesions PSMA-PET or no more than three lesions by conventional imaging; the subject has not received an ADT or ARPI within one year of administration of the isolated anti-hK2/anti-CD3 bispecific antibody or bispecific fragment thereof; and the subject has non-castrate levels of testosterone of greater than 150 ng/mL.

In some embodiments, the prostate cancer (prior to the administering of a therapeutically effective amount of an isolated anti-hK2/anti-CD3 bispecific antibody or a bispecific fragment thereof) is a metastatic hormone sensitive prostate cancer (mHSPC). In some embodiments, the mHSPC is a mHSPC with no (evidence of) metastasis to visceral organs. In some embodiments, the mHSPC is a mHSPC with metastasis in no more than five (distinct) locations in the patient's body. In some embodiments, the mHSPC is a mHSPC with no more than 5 isocenters (lesions within radiation field for ablative metastasis-directed radiotherapy), for example with no more than 5 cancerous lesions. In some embodiments, the mHSPC is a mHSPC with metastasis in no more than five (distinct) locations in the patient's body, more particularly a mHSPC with no more than 5 isocenters (lesions within radiation field for ablative metastasis-directed radiotherapy), for example with no more than 5 cancerous lesions; and is a mHSPC with no (evidence of) metastasis to visceral organs. In some embodiments, the mHSPC is a mHSPC with non-castrate levels of testosterone (e.g., at study entry), for example with levels of testosterone >150 ng/dL (e.g., due to testosterone supplementation prior to the administering of a therapeutically effective amount of an isolated anti-hK2/anti-CD3 bispecific antibody or a bispecific fragment thereof). In some embodiments, the (mHSPC) patient does not have undergone orchiectomy. In some embodiments, the method further comprises administering metastasis-directed radiotherapy (MDR) to the (mHSPC) patient. In some embodiments, the subject has advanced mCRPC and has received all the life-prolonging therapies for which he is eligible.

In some embodiments, the subject has advanced mCRPC and is a subject for whom clinical trial is the best treatment option.

In some embodiments, the subject has mCRPC in the late-line setting.

In some embodiments, concurrent use of any other anticancer treatment (including non-palliative radiotherapy) or investigational agent, or prior anticancer treatments (including non-palliative radiotherapy) is discontinued for at least two weeks prior to the subject being administered with the isolated anti-hK2/anti-CD3 bispecific antibody or bispecific fragment thereof.

In some embodiments, the subject has one or more of the following:
(a) Eastern Cooperative Oncology Group (ECOG) performance status of 0 or 1;
(b) Hematology laboratory parameters within the following ranges, independent of transfusion within 7 days, or growth factors administered within three weeks prior to the subject being administered with the isolated anti-hK2/anti-CD3 bispecific antibody or bispecific fragment thereof:
   (i) Hemoglobin ≥9.0 g/dL
   (ii) Absolute neutrophil count ≥1.5×109/L
   (iii) Platelet count ≥75×109/L; and/or
(c) Clinical chemistry laboratory parameters within the following range prior to the subject being administered with the isolated anti-hK2/anti-CD3 bispecific antibody or bispecific fragment thereof:
   (i) Alanine aminotransferase (ALT) and aspartate aminotransferase (AST)≤2.5×ULN (or ≤4×ULN if the subject has tumor involvement in the liver)
   (ii) Serum albumin ≥3.0 g/dL
   (iii) Serum total bilirubin ≤1.5×ULN (or ≤1.5×ULN conjugated bilirubin if the subject has congenital bilirubinemia, such as Gilbert's syndrome)
   (iv) Estimated or measured glomerular filtration rate (GFR)≥50 mL/min/1.73 m2.
      In some embodiments, the subject has clinical laboratory parameters within the following ranges within 7 days prior to randomization:
(i) Hemoglobin >8.0 g/dL
(ii) Absolute neutrophil count (ANC)>1.0×109/L
(iii) Platelet count >75×109/L
(iv) ALT and AST≤3×ULN.
(v) Serum total bilirubin ≤1.5×ULN; participants with congenital bilirubinemia, such as Gilbert's syndrome, may enroll if conjugated bilirubin is >1.5×ULN.

In some embodiments, the subject:
a) Has histologically confirmed adenocarcinoma of the prostate as defined by PCWG3 criteria.
b) Has a disease that is metastatic to bone, lymph node, or both without evidence of metastasis to visceral organs at the time of screening
c) Has progressed on at least 1 ARPI and is unlikely to benefit from retreatment with another ARPI.
d) Has been previously treated with at least 2 previous taxane regimens. A taxane regimen is defined as a minimum exposure of 2 cycles of a taxane. If a patient has received only 1 taxane regimen, the patient is eligible if:
   (i) Cabazitaxel is not available.
   (ii) The patient's physician deems the participant unsuitable to receive a second taxane regimen due to toxicity risk of prior intolerance
e) Has been previously treated with at least 1 dose of PSMA-targeted lutetium radioligand therapy (eg, lutetium Lu-177 vipivotide tetraxetan), unless one of the following applies:
   (i) PSMA-targeted lutetium radioligand therapy is unavailable, not accessible, or not clinically indicated
   (ii) The patient's physician deems the participant unsuitable to receive PSMA-targeted lutetium radioligand therapy.
f) Has been previously treated with a PARPi, if participant has a known germline or somatic BRCA mutation and treatment is available.
g) Has clinical laboratory parameters within the following ranges within 7 days prior to randomization. Transfusion or growth factor usage within 14 days of randomization is not allowed.
   (i) Hemoglobin >8.0 g/dL
   (ii) Absolute neutrophil count (ANC)>1.0×109/L
   (iii) Platelet count >75×109/L
   (iv) ALT and AST≤3 xULN.
   (v) Serum total bilirubin ≤1.5×ULN; participants with congenital bilirubinemia, such as Gilbert's syndrome, may enroll if conjugated bilirubin is >1.5×ULN.

In some embodiments, the next best treatment option for the patient is a clinical trial in the opinion of the investigator.

The methods of treatment disclosed herein may provide on or more of the following therapeutic effects in the subject.

In some embodiments, the method provides a decrease in the serum Prostate-Specific Antigen (PSA) level of the subject, wherein the decrease is relative to the PSA level of the subject prior to the administration of the isolated anti-hK2/anti-CD3 bispecific antibody or bispecific fragment thereof. In certain such embodiments the PSA level decreases by 50% or more compared to the PSA level of the subject prior to the administration of the isolated anti-hK2/anti-CD3 bispecific antibody or bispecific fragment thereof.

In some embodiments, the method provides for no disease progression in the subject, wherein disease progression is assessed according to the Prostate Cancer Working Group 3 (PCWG3) Criteria (Scher et al. 2016 Journal of Clinical Oncology 34(12): 1402-1418).

In some embodiments, the method provides a Partial Response or better according to RECIST version 1.1 response criteria (Eisenhauer et al. 2009 European Journal of Cancer 45: 228-247) without evidence of bone progression according to PCWG3 in the subject.

In some embodiments, the method provides an improvement in the overall survival (OS).

The methods of treatment disclosed herein may also reduce adverse events or side effects from administration of the anti-hk2/anti-CD3 antibody.

In some embodiments, no Adverse Event of Grade 3 or higher (according to the National Cancer Institute Common Terminology Criteria for Adverse Events (NCI-CTCAE) version 5.0) is observed in the subject.

In some embodiments, no event of Injection Site Reaction (ISR) of Grade 3 or higher (according to the National Cancer Institute Common Terminology Criteria for Adverse Events (NCI-CTCAE) version 5.0) is observed in the subject.

In some embodiments, no event of Fatigue of Grade 3 or higher (according to the National Cancer Institute Common Terminology Criteria for Adverse Events (NCI-CTCAE) version 5.0) is observed in the subject.

In some embodiments, no event of Nausea of Grade 2 or higher (according to the National Cancer Institute Common Terminology Criteria for Adverse Events (NCI-CTCAE) version 5.0) is observed in the subject.

In some embodiments, no events of Anemia of Grade 3 or higher (according to the National Cancer Institute Common Terminology Criteria for Adverse Events (NCI-CTCAE) version 5.0) is observed in the subject.

In some embodiments, no event of Cytokine Release Syndrome (CRS) (according to the American Society for Transplantation and Cellular Therapy (ASTCT) guidelines) is observed in the subject.

In some embodiments, no event of Cytokine Release Syndrome (CRS) of Grade 3 or higher (according to the American Society for Transplantation and Cellular Therapy (ASTCT) guidelines) is observed in the subject.

In some embodiments, no event of pyrexia (fever) of Grade 2 or higher (according to the National Cancer Institute Common Terminology Criteria for Adverse Events (NCI-CTCAE) version 5.0) is observed in the subject.

The number of Adverse Events may also be measured across a cohort of patients. Accordingly, in some embodiments, Adverse Events of Grade 3 or higher (according to the National Cancer Institute Common Terminology Criteria for Adverse Events (NCI-CTCAE) version 5.0) are observed in 20% or fewer of subjects in a cohort of at least 20 subjects, for example 15% or fewer of subjects in a cohort of at least 20 subjects.

In some embodiments, events of Injection Site Reactions (ISR) (according to the National Cancer Institute Common Terminology Criteria for Adverse Events (NCI-CTCAE) version 5.0) are observed in 10% or fewer subjects in a cohort of at least 20 subjects, for example 5% or fewer of subjects in a cohort of at least 20 subjects.

In some embodiments, events of Fatigue (according to the National Cancer Institute Common Terminology Criteria for Adverse Events (NCI-CTCAE) version 5.0) are observed in 30% or fewer of subjects in a cohort of at least 20 subjects, for example 25% or fewer of subjects in a cohort of at least 20 subjects, such as 20% or fewer of subjects in a cohort of at least 20 subjects.

In some embodiments, events of Nausea (according to the National Cancer Institute Common Terminology Criteria for Adverse Events (NCI-CTCAE) version 5.0) are observed in 20% or fewer of subjects in a cohort of at least 20 subjects, for example 15% or fewer of subjects in a cohort of at least 20 subjects.

In some embodiments events of Anemia (according to the National Cancer Institute Common Terminology Criteria for Adverse Events (NCI-CTCAE) version 5.0) are observed in 20% or fewer of subjects in a cohort of at least 20 subjects, for example 15% or fewer of subjects in a cohort of at least 20 subjects, such as 10% or fewer of subjects in a cohort of at least 20 subjects.

In some embodiments, events of Cytokine Release Syndrome (CRS) (according to the American Society for Transplantation and Cellular Therapy (ASTCT) guidelines) are observed in 15% or fewer of subjects in a cohort of at least 20 subjects, for example 10% or fewer of subjects in a cohort of at least 20 subjects.

In some embodiments, events of Cytokine Release Syndrome (CRS) of Grade 3 or higher (according to the American Society for Transplantation and Cellular Therapy (ASTCT) guidelines) are observed in 15% or fewer of subjects in a cohort of at least 20 subjects, for example 10% or fewer of subjects in a cohort of at least 20 subjects.

In some embodiments, events of Cytokine Release Syndrome (CRS) of Grade 2 or higher (according to the American Society for Transplantation and Cellular Therapy (ASTCT) guidelines) are observed in 15% or fewer of subjects in a cohort of at least 20 subjects, for example 10% or fewer of subjects in a cohort of at least 20 subjects.

In some embodiments, events of constipation (according to the National Cancer Institute Common Terminology Criteria for Adverse Events (NCI-CTCAE) version 5.0) are observed in 15% or fewer subjects in a cohort of at least 20 subjects.

In some embodiments, events of back pain (according to the National Cancer Institute Common Terminology Criteria for Adverse Events (NCI-CTCAE) version 5.0) are observed in 20% or fewer subjects in a cohort of at least 20 subjects, for example in 15% or fewer subjects in a cohort of at least 20 subjects.

In some embodiments, events of arthralgia (according to the National Cancer Institute Common Terminology Criteria for Adverse Events (NCI-CTCAE) version 5.0) are observed in 15% or fewer subjects in a cohort of at least 20 subjects, for example in 10% or fewer subjects in a cohort of at least 20 subjects.

In some embodiments, the method does not induce a Dose Limiting Toxicity (DLT) in the subject.

A reduction in adverse events may mean that patients may be treated as outpatients, and not require hospitalization.

In some embodiments, the subject does not have one or more of the following:
(a) active CNS involvement, except treated, stable and asymptomatic brain metastases requiring <10 mg prednisone or equivalent within two weeks prior to the first dose of study treatment are permitted;
(b) toxicity related to prior anticancer therapy which has not returned to Grade ≤1 or baseline levels;
(c) prior treatment with KLK2-targeted therapy;
(d) received immunosuppressive doses of systemic medications, such as glucocorticoids (doses >10 mg/day prednisone or equivalent) within 3 days prior to administration of the isolated anti-hK2/anti-CD3 bispecific antibody or bispecific fragment thereof;
(e) received or plans to receive any live, attenuated vaccine within four weeks before the first dose of study treatment or within 4 weeks after the last dose of study drug;
(f) malignancy diagnosis other than the disease under study within two years prior to the first dose of study drug, except for squamous and basal cell carcinoma of the skin, non-muscle invasive bladder cancer, or any malignancy considered cured or has minimal risk of recurrence within 1 year of first dose of study drug;
(g) solid organ or bone marrow transplantation;
(h) venous thromboembolic events (e.g., pulmonary embolism) within 1 month prior to the first dose of study treatment; uncomplicated (Grade ≤2) deep vein thrombosis is not considered exclusionary;
(i) active autoimmune disease within the 12 months prior to signing consent that requires systemic immunosuppressive medications;
(j) active infection or condition that requires treatment with systemic antibiotics within 7 days prior to the first dose of the isolated anti-hK2/anti-CD3 bispecific antibody or bispecific fragment thereof;
(k) any of the following within 6 months prior to signature of informed consent:
   i. Myocardial infarction;
   ii. Severe or unstable angina;
   iii. Clinically significant ventricular arrhythmias;
   iv. Congestive heart failure (according to New York Heart Association [NYHA] class II to IV);
   v. Transient ischemic attack;
   vi. Cerebrovascular accident;
(l) clinically significant pulmonary compromise, particularly a requirement for supplemental oxygen use (>2 L/min by nasal cannula) to maintain adequate oxygenation;
(m) active or chronic hepatitis B virus (HBV) or hepatitis C virus (HCV) infection according to a local laboratory range;
(n) known positive test result for human immunodeficiency virus (unless stable on antiretroviral therapy with undetectable viral load and CD4 count above 200 cells/mm³);
(o) known allergies, hypersensitivity, or intolerance to the isolated anti-hK2/anti-CD3 bispecific antibody or bispecific fragment thereof or its excipients;
(p) any serious underlying medical conditions or other issue that would impair the ability of the participant to receive or tolerate the planned treatment at the investigational site, to understand the informed consent, or any condition for which, in the opinion of the investigator, participation would not be in the best interest of the participant (e.g. compromise the well-being) or that could prevent, limit, or confound the protocol-specified assessments; and/or
(q) major surgery (e.g. requiring general anesthesia) within two weeks before first dose of the isolated anti-hK2/anti-CD3 bispecific antibody or bispecific fragment thereof or has not recovered from surgery.

The methods of the invention may involve administering the anti-hk2/anti-CD3 bispecific alone and not in combination with other therapeutic agents.

Accordingly, in some embodiments, the isolated anti-hK2/anti-CD3 bispecific antibody or a bispecific fragment thereof, is administered as a monotherapy.

In some embodiments, the isolated anti-hK2/anti-CD3 bispecific antibody or a bispecific fragment thereof, is not administered to the subject in combination with a PSMAxCD28 bispecific antibody, wherein the isolated anti-PSMA/anti-CD28 bispecific antibody comprises a binding domain that binds specifically to PSMA, and a binding domain that binds specifically to CD28, wherein the PSMA binding domain comprises a HCDR1, a HCDR2 and a HCDR3 of a heavy chain variable region of SEQ ID NO: 63, and a LCDR1, a LCDR2, and a LCDR3 of a light chain variable region of SEQ ID NO: 64, and the combination dose of the anti-PSMA/anti-CD28 bispecific antibody is 0.5-2000 mg per administration.

In some embodiments, the method does not comprise administering an androgen receptor pathway inhibitor (ARPI) or an anti-cancer chemotherapeutic agent to the subject, such as taxane.

In some embodiments, the method consists essentially of administering a therapeutically effective amount of an isolated anti-hK2/anti-CD3 bispecific antibody or a bispecific fragment thereof, to the subject by intravenous (IV) infusion and optionally one or more step-up doses.

The methods of the invention may also involve administering the anti-hk2/anti-CD3 bispecific in combination with other therapeutic agents. Accordingly, in some embodiments, the method comprises administering to the subject one or more additional therapeutic agent. In certain such embodiments, the one or more additional therapeutic agent is one or more additional anti-cancer therapies. In other embodiments, the one or more additional therapeutic agents is one or more immune modulating agents.

### Compositions

The anti-hK2/anti-CD3 bispecific antibodies or bispecific fragment thereof disclosed herein can be administered in pharmaceutical compositions that include an effective amount of the bispecific antibody or bispecific fragment thereof, and one or more pharmaceutically acceptable carriers or excipients, which can be formulated by methods known to those skilled in the art.

Acceptable carriers and excipients in the pharmaceutical compositions are nontoxic to recipients at the dosages and concentrations employed. Acceptable carriers and excipients may include buffers, antioxidants, preservatives, polymers, amino acids, and carbohydrates. In some embodiments, the pharmaceutical composition further comprises one or more excipients. In some embodiments, the one or more excipients include, but are not limited to a buffering agent, a sugar, a surfactant, a chelator, or any combination thereof.

Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (2nd ed.) Taylor & Francis Group, CRC Press (2006).

In some embodiments, the anti-hK2/anti-CD3 bispecific antibodies or bispecific fragment thereof and other anti-cancer drugs useful for a method of the application can be packaged in one or more kits, which can optionally contain instructions for use.

### Embodiments

The disclosure provided herein also provides the following non-limiting embodiments.

1. A method of treating prostate cancer in a human subject in need thereof, wherein the method comprises administering a therapeutically effective amount of an isolated anti-hK2/anti-CD3 bispecific antibody or a bispecific fragment thereof to the subject at a treatment dose by intravenous (IV) infusion, wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18.

2. The method of embodiment 1, wherein the treatment dose is at least about 75 mg per administration.

3. The method of embodiment 2, wherein the treatment dose is between about 75 mg and about 900 mg per administration, for example about 75 mg, about 100 mg, about 150 mg, about 200 mg, about 250 mg, about 300 mg, about 400 mg, about 500 mg, about 600 mg, about 700 mg, about 800 mg, or about 900 mg per administration.

4. The method of embodiment 2 or embodiment 3, wherein the treatment dose is about 75 mg per administration.

5. The method of any one of embodiments 1-3, wherein the treatment dose is at least about 150 mg per administration.

6. The method of embodiment 5, wherein the treatment dose is between about 150 mg and about 900 mg per administration, for example about 150 mg, about 200 mg, about 250 mg, about 300 mg, about 400 mg, about 500 mg, about 600 mg, about 700 mg, about 800 mg, or about 900 mg per administration.

7. The method of embodiment 5 or embodiment 6, wherein the treatment dose is about 150 mg per administration.

8. The method of any one of embodiments 1-3, and 5-6, wherein the treatment dose is at least about 200 mg per administration.

9. The method of embodiment 8, wherein the treatment dose is between about 200 mg and about 900 mg per administration, for example about 200 mg, about 250 mg, about 300 mg, about 400 mg, about 500 mg, about 600 mg, about 700 mg, about 800 mg or about 900 mg per administration.

10. The method of embodiment 8 or embodiment 9, wherein the treatment dose is about 200 mg per administration.

11. The method of embodiment 8 or embodiment 9 wherein the treatment dose is about 250 mg per administration.

12. The method of any one of embodiments 1-3, 5-6, and 8-9, wherein the treatment dose is at least about 300 mg per administration.

13. The method of embodiment 12, wherein the treatment dose is between about 300 mg and about 900 mg per administration, for example about 300 mg, about 400 mg, about 500 mg, about 600 mg, about 700 mg, about 800 mg or about 900 mg per administration.

14. The method of embodiment 12 or embodiment 13, wherein the treatment dose is about 300 mg per administration.

15. The method of any one of embodiments 1-3, 5-6, 8-9, and 12-13, wherein the treatment dose is at least about 600 mg per administration.

16. The method of embodiment 15, wherein the treatment dose is between about 600 mg and about 900 mg per administration, for example, about 600 mg, about 700 mg, about 800 mg or about 900 mg per administration.

17. The method of embodiment 16, wherein the treatment dose is about 600 mg per administration.

18. The method of embodiment 16, wherein the treatment dose is about 900 mg per administration.

19. The method of any one of embodiments 1-18, wherein the treatment dose is administered at a flat dose.

20. The method of any one of embodiments 1-19, wherein the treatment dose is administered once every 1 to 6 weeks, such as once every 1, 2, 3, 4, 5, or 6 weeks.

21. The method of any one of embodiments 20, wherein the treatment dose is administered once every three weeks.

22. The method of any one of embodiments 20, wherein the treatment dose is administered once every six weeks.

23. The method of any one of embodiments 1-22, wherein the treatment dose is administered for at least two cycles (for example, two, three, four, five, ten, fifteen, twenty or more cycles).

24. The method of embodiment 23, wherein the treatment dose is administered for at least a year, or throughout the lifespan of the subject.

25. The method of any one of embodiments 1-24, wherein the treatment dose is administered by intravenous (IV) infusion over a period of at least about one hour, for example wherein the treatment dose is administered by IV infusion over a period of about one hour.

26. The method of any one of embodiments 1-24, wherein the treatment dose is administered by intravenous (IV) infusion over a period of at least about 30 minutes, for example wherein the treatment dose is administered by IV infusion over a period of about 30 minutes.

27. The method of any one of embodiments 1-26, wherein the first treatment dose is administered by intravenous (IV) infusion over a period of at least about one hour, for example wherein the treatment dose is administered by IV infusion over a period of about one hour and any subsequent treatment dose is administered by intravenous (IV) infusion over a period of at least about 30 minutes, for example wherein the treatment dose is administered by IV infusion over a period of about 30 minutes.

28. The method of any one of embodiments 1-27, wherein the method does not comprise administering a step-up dose prior to the administration of the treatment dose.

29. The method of any one of embodiments 1-28, wherein the method comprises administering to the human subject one or more step-up doses (for example, one or two step-up doses) of the anti-hK2/anti-CD3 bispecific antibody or a bispecific fragment thereof, wherein the one or more step-up doses are administered prior to the treatment dose and wherein the one or more step-up doses are not higher than the treatment dose.

30. The method of embodiment 29, wherein the method comprises administering to the subject a step-up dose of between about 2 mg and about 5 mg per administration, for example about 2 mg, about 2.5 mg, about 3 mg, about 3.5 mg, about 4 mg, about 4.5 mg or about 5 mg per administration, prior to the treatment dose.

31. The method of embodiment 30, wherein the step-up dose is about 3.5 mg per administration.

32. The method of any one of embodiments 29-31, wherein the method comprises administering to the subject a step-up dose of between about 5 mg and about 15 mg per administration, for example about 5 mg, about 10 mg, or about 15 mg per administration, prior to the treatment dose.

33. The method of embodiment 32, wherein the step-up dose is about 10 mg per administration.

34. The method of any one of embodiments 29-33, wherein the method comprises administering to the subject a step-up dose of between about 15 mg and about 25 mg per administration, for example about 15 mg, about 16 mg, about 17 mg, about 18 mg, about 19 mg, about 20 mg, about 21 mg, about 22 mg, about 23 mg, about 24 mg, or about 25 mg per administration, prior to the treatment dose.

35. The method of embodiment 34, wherein the step-up dose is about 18 mg per administration.

36. The method of any one of embodiments 29-35, wherein the method comprises administering to the subject a step-up dose of between about 200 and about 400 mg per administration, for example about 200 mg, about 300 mg or about 400 mg per administration, prior to the treatment dose.

37. The method of embodiment 36, wherein the step-up dose is about 300 mg per administration.

38. The method of any one of embodiments 29-37, wherein the step-up dose is administered 3-8 days, for example 3, 4, 5, 6, 7 or 8 days prior to the administration of the treatment dose.

39. The method of embodiment 38, wherein the administration of the step-up dose is 3 days prior to the administration of the treatment dose.

40. The method of embodiment 38, wherein the administration of the step-up dose is 7 days prior to the administration of the treatment dose.

41. The method of any one of embodiments 29-40, wherein the one or more step-up doses are administered via intravenous (IV) infusion.

42. The method of any one of embodiments 29-41, wherein the one or more step-up doses are administered at a flat dose.

43. The method of any one of embodiments 1-27 and 29-42, wherein the method comprises administering to the subject:
i) a step-up dose of the isolated anti-hK2/anti-CD3 bispecific antibody or a bispecific fragment thereof; and
ii) a treatment dose of the isolated anti-hK2/anti-CD3 bispecific antibody or a bispecific fragment thereof,
   wherein the step-up dose is administered once.

44. The method of any one of embodiments 1-27 and 29-42, wherein the method comprises administering to the subject a first step-up dose and a second step-up dose of the isolated anti-hK2/anti-CD3 bispecific antibody or a bispecific fragment thereof, wherein the first step-up dose is administered prior to the second step-up dose, and the second step-up dose is administered prior to the treatment dose.

45. The method of embodiment 44, wherein the second step-up dose is greater than the first step-up dose.

46. The method of embodiment 44 or embodiment 45, wherein the first step-up dose is between about 2 mg and about 5 mg per administration, for example about 2 mg, about 2.5 mg, about 3 mg, about 3.5 mg, about 4 mg, about 4.5 mg or about 5 mg per administration.

47. The method of embodiment 46, wherein the first step-up dose is about 3.5 mg per administration.

48. The method of embodiment 44 or embodiment 45, wherein the first step-up dose is between about 5 mg and about 15 mg per administration, for example about 5 mg, about 10 mg, or about 15 mg per administration.

49. The method of embodiment 48, wherein the first step-up dose is about 10 mg per administration.

50. The method of any one of embodiments 44-49, wherein the second step-up dose is between about 5 mg and about 15 mg per administration, for example about 5 mg, about 10 mg, or about 15 mg per administration.

51. The method of embodiment 50, wherein the second step-up dose is about 10 mg per administration.

52. The method of any one of embodiments 44-49, wherein the second step-up dose is between about 15 mg and about 25 mg per administration, for example about 15 mg, about 16 mg, about 17 mg, about 18 mg, about 19 mg, about 20 mg, about 21 mg, about 22 mg, about 23 mg, about 24 mg, or about 25 mg per administration.

53. The method of embodiment 52, wherein the second step-up dose is about 18 mg per administration.

54. The method of any one of embodiments 44-49, wherein the second step-up dose is between about 200 mg and about 400 mg per administration, for example about 200 mg, about 300 mg or about 400 mg per administration.

55. The method of embodiment 52, wherein the second step-up dose is about 300 mg per administration.

56. The method of any one of embodiments 44-55, wherein the first step-up dose is administered 3-8 days, for example 3, 4, 5, 6, 7 or 8 days prior to the administration of the second step-up dose.

57. The method of embodiment 56, wherein the first step-up dose is administered 3 days prior to the administration of the second step-up dose.

58. The method of embodiment 56, wherein the first step-up dose is administered 7 days prior to the administration of the second step-up dose.

59. The method of any one of embodiments 44-58, wherein the second step-up dose is administered 3-8 days, for example 3, 4, 5, 6, 7 or 8 days prior to the administration of the treatment dose.

60. The method of embodiment 59, wherein the second step-up dose is administered 3 days prior to the administration of the treatment dose.

61. The method of embodiment 59, wherein the second step-up dose is administered 7 days prior to the administration of the treatment dose.

62. The method of any one of embodiments 44-61, wherein the method comprises administering to the subject:
i) a first step-up dose of the isolated anti-hK2/anti-CD3 bispecific antibody or a bispecific fragment thereof,
ii) a second step-up dose of the isolated anti-hK2/anti-CD3 bispecific antibody or a bispecific fragment thereof; and
iii) a treatment dose of the isolated anti-hK2/anti-CD3 bispecific antibody or a bispecific fragment thereof,
   wherein the first step-up dose is administered once, and the second step-up dose is administered once.

63. The method of any one of embodiments 29-62, wherein the first step-up dose is administered via intravenous (IV) infusion.

64. The method of any one of embodiments 44-63, wherein the second step-up dose is administered via intravenous (IV) infusion.

65. The method of any one of embodiments 29-64, wherein the first step-up dose is administered at a flat dose.

66. The method of any one of embodiments 29-65, wherein the second step-up dose is administered at a flat dose.

67. The method of any one of embodiments 29-66, wherein the first step-up dose is administered by intravenous (IV) infusion over a period of at least about one hour, for example wherein the treatment dose is administered by IV infusion over a period of about one hour.

68. The method of any one of embodiments 44-67, wherein the second step-up dose is administered by intravenous (IV) infusion over a period of at least about 30 minutes, for example wherein the treatment dose is administered by IV infusion over a period of about 30 minutes.

69. The method of any one of embodiment 67 or embodiment 68, wherein the treatment dose is administered by intravenous (IV) infusion over a period of at least about 30 minutes, for example wherein the treatment dose is administered by IV infusion over a period of about 30 minutes.

70. The method of any one of embodiments 44 and 56-69, wherein the method comprises administering to the subject a first step-up dose and a second step-up dose of the isolated anti-hK2/anti-CD3 bispecific antibody or a bispecific fragment thereof, wherein the first step-up dose is administered prior to the second step-up dose, and wherein the second step-up dose is administered prior to the treatment dose, wherein:
(a) the first step-up dose is about 3.5 mg;
(b) the second step-up dose is about 18 mg; and
(c) the treatment dose is about 300 mg.

71. The method of embodiment 70, wherein the first step-up dose is administered 7 days prior to the second step-up dose.

72. The method of embodiment 70 or embodiment 71, wherein the second step-up dose is administered 7 days prior to the treatment dose.

73. The method of any one of embodiments 70-72, wherein the treatment dose is administered once every six weeks.

74. The method of any one of embodiments 70-72, wherein the treatment dose is administered once every three weeks.

75. The method of any one of embodiments 44 and 56-69, wherein the method comprises administering to the subject a first step-up dose and a second step-up dose of the isolated anti-hK2/anti-CD3 bispecific antibody or a bispecific fragment thereof, wherein the first step-up dose is administered prior to the second step-up dose, and wherein the second step-up dose is administered prior to the treatment dose, wherein:
(a) the first step-up dose is about 3.5 mg;
(b) the second step-up dose is about 18 mg; and
(c) the treatment dose is about 600 mg.

76. The method of embodiment 75, wherein the first step-up dose is administered about 7 days prior to the second step-up dose.

77. The method of embodiment 75 or embodiment 76, wherein the second step-up dose is administered about 7 days prior to the treatment dose.

78. The method of any one of embodiments 75-77, wherein the treatment dose is administered once every six weeks.

79. The method of any one of embodiments 75-78, wherein the treatment dose is administered once every three weeks.

80. The method of any one of embodiments 29 and 38-43, wherein the method comprises administering to the subject a step-up dose of the isolated anti-hK2/anti-CD3 bispecific antibody or a bispecific fragment thereof, wherein the step-up dose is administered prior to the treatment dose, wherein:
(a) the step-up dose is about 10 mg; and
(b) the treatment dose is about 300 mg.

81. The method of embodiment 80, wherein the first step-up dose is administered 7 days prior to the treatment dose.

82. The method of embodiment 80 or embodiment 81, wherein the treatment dose is administered once every six weeks.

83. The method of any one of embodiments 80-82, wherein the treatment dose is administered once every three weeks.

84. The method of any one of embodiments 29 and 38-43, wherein the method comprises administering to the subject a step-up dose of the isolated anti-hK2/anti-CD3 bispecific antibody or a bispecific fragment thereof, wherein the step-up dose is administered prior to the treatment dose, wherein:
(a) the step-up dose is about 10 mg; and
(b) the treatment dose is about 600 mg.

85. The method of embodiment 84, wherein the first step-up dose is administered about 7 days prior to the treatment dose.

86. The method of embodiment 84 or embodiment 85, wherein the treatment dose is administered once every six weeks.

87. The method of any one of embodiments 84-86, wherein the treatment dose is administered once about every three weeks.

88. The method of any one of embodiments 44 and 56-69, wherein the method comprises administering to the subject a first step-up dose and a second step-up dose of the isolated anti-hK2/anti-CD3 bispecific antibody or a bispecific fragment thereof, wherein the first step-up dose is administered prior to the second step-up dose, and wherein the second step-up dose is administered prior to the treatment dose, wherein:
(a) the first step-up dose is about 10 mg;
(b) the second step-up dose is about 300 mg; and
(c) the treatment dose is about 900 mg.

89. The method of embodiment 88, wherein the first step-up dose is administered 7 days prior to the second step-up dose.

90. The method of embodiment 88 or embodiment 89, wherein the second step-up dose is administered 7 days prior to the treatment dose.

91. The method of any one of embodiments 88-90, wherein the treatment dose is administered once every six weeks.

92. The method of any one of embodiments 44 and 56-69, wherein the method comprises administering to the subject a first step-up dose and a second step-up dose of the isolated anti-hK2/anti-CD3 bispecific antibody or a bispecific fragment thereof, wherein the first step-up dose is administered prior to the second step-up dose, and wherein the second step-up dose is administered prior to the treatment dose, wherein:
(a) the first step-up dose is about 10 mg;
(b) the second step-up dose is about 300 mg; and
(c) the treatment dose is about 600 mg.

93. The method of embodiment 92, wherein the first step-up dose is administered 7 days prior to the second step-up dose.

94. The method of embodiment 92 or embodiment 93, wherein the second step-up dose is administered 7 days prior to the treatment dose.

95. The method of any one of embodiments 92-94, wherein the treatment dose is administered once every six weeks.

96. The method of any one of embodiments 1-95, wherein the first domain that binds hK2 and/or the second domain that binds CD3 comprise a scFv, a (scFv)2, a Fv, a Fab, or a F(ab′)2.

97. The method of any one of embodiments 1-96, wherein the first domain that binds hK2 comprises a Fab and the second domain that binds CD3 comprises a scFv.

98. The method of embodiment 96 or embodiment 97, wherein the scFv comprises, from N- to C-terminus, a VH, a first linker (L1) and a VL (VH-L1-VL) or the VL, the L1 and the VH (VL-L1-VH).

99. The method of embodiment 98, wherein L1 comprises:
(a) about 5-50 amino acids;
(b) about 5-40 amino acids;
(c) about 10-30 amino acids; or
(d) about 10-20 amino acids.

100. The method of embodiment 99, wherein the L1 comprises the amino acid sequence of SEQ ID NO: 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52 or 53, for example wherein the L1 comprises the amino acid sequence of SEQ ID NO: 20.

101. The method of any one of embodiments 1-100, wherein the first domain that binds hK2 is conjugated to a first immunoglobulin (Ig) heavy chain constant region, or a fragment of the first Ig heavy chain constant region, and the second domain that binds CD3ε is conjugated to a second immunoglobulin (Ig) heavy chain constant region, or a fragment of the second Ig heavy chain constant region.

102. The method of embodiment 101, wherein the first Ig heavy chain constant region, or the fragment of the first Ig heavy chain constant region, and the second Ig heavy chain constant region, or the fragment of the second Ig heavy chain constant region, is an IgG1 isotype.

103. The method of any one of embodiments 101 or 102, wherein the antibody or the bispecific fragment thereof comprises the following amino acid mutations:
(a) L234A_L235A_D265S_T350V_T366L_K392L_T394W in the first Ig heavy chain constant region, or the fragment of the first Ig heavy chain constant region, and
(b) L234A_L235A_D265S_T350V_L351Y_F405A_Y407V in the second Ig heavy chain constant region, or the fragment of the second Ig heavy chain constant region,
   wherein numbering of the amino acid mutations is of the EU index.

104. The method of any one of embodiments 101-103, wherein: the first Ig heavy chain constant region comprises an amino acid sequence which is at least 80% (e.g. at least 85%, at least 90%, at least 95%, at least 99% or 100%) identical to SEQ ID NO: 54 and the second Ig heavy chain constant region comprises an amino acid sequence which is at least 80% (e.g. at least 85%, at least 90%, at least 95%, at least 99% or 100%) identical to SEQ ID NO: 55.

105. The method of embodiment 104, wherein the first Ig heavy chain constant region comprises an amino acid sequence which is at least 95% (e.g. at least 99% or 100%) identical to SEQ ID NO: 54 and the second Ig heavy chain constant region comprises an amino acid sequence which is at least 95% (e.g. at least 99% or 100%) identical to SEQ ID NO: 55.

106. The method of any one of embodiments 102-105, wherein the first Ig heavy chain constant region, or the fragment of the first Ig heavy chain constant region, comprises an amino acid sequence of SEQ ID NO: 54.

107. The method of any one of embodiments 102-106, wherein the second Ig heavy chain constant region, or the fragment of the second Ig heavy chain constant region, comprises an amino acid sequence of SEQ ID NO: 55.

108. The method of any one of embodiments 1-107 wherein the first domain that binds hK2 is conjugated to a first immunoglobulin (Ig) light chain constant region, or a fragment of the first Ig light chain constant region.

109. The method of embodiment 108, wherein the first immunoglobulin (Ig) light chain constant region, or the fragment of the first Ig light chain constant region, comprises an amino acid sequence which is at least 80% (e.g. at least 85%, at least 90%, at least 95%, at least 99% or 100%) identical to SEQ ID NO: 56.

110. The method of any one of embodiments 1-109, wherein the isolated anti-hK2/anti-CD3 antibody or a bispecific fragment thereof, comprises a HC1 which is at least 80% (for example at least 95%) identical to the HC1 of SEQ ID NO: 57, a LC1 that is at least 80% (for example at least 95%) identical to the LC1 of SEQ ID NO: 10 and a HC2 which is at least 80% (for example at least 95%) identical to the HC2 of SEQ ID NO: 58.

111. The method of any one of embodiments 1-110, wherein the isolated anti-hK2/anti-CD3 antibody or a bispecific fragment thereof, comprises a HC1 of SEQ ID NO: 57, a LC1 of SEQ ID NO: 10 and a HC2 of SEQ ID NO: 58.

112. The method of embodiment 110 or embodiment 111 wherein the isolated anti-hK2/anti-CD3 antibody or a bispecific fragment thereof, comprises a lysine at the C-terminus of HC1 and HC2

113. The method of any one of embodiments 1-109, wherein the isolated anti-hK2/anti-CD3 antibody or a bispecific fragment thereof, comprises a HC1 which is at least 80% (for example at least 95%) identical to the HC1 of SEQ ID NO: 9, a LC1 that is at least 80% (for example at least 95%) identical to the LC1 of SEQ ID NO: 10 and a HC2 which is at least 80% (for example at least 95%) identical to the HC2 of SEQ ID NO: 19.

114. The method of any one of embodiments 1-113, wherein the isolated anti-hK2/anti-CD3 antibody or a bispecific fragment thereof, comprises a HC1 of SEQ ID NO: 9, a LC1 of SEQ ID NO: 10 and a HC2 of SEQ ID NO: 19.

115. The method of any one of embodiments 1-114, wherein the human subject is 18 years of age or older.

116. The method of any one of embodiments 1-115, wherein the prostate cancer is histologically confirmed adenocarcinoma of the prostate, as defined by Prostate Cancer Working Group 3 (PCWG3) criteria (Scher et al. 2016 Journal of Clinical Oncology 34(12): 1402-1418).

117. The method of embodiment 116, wherein the prostate cancer is prostate adenocarcinoma that has small cell or neuroendocrine (NE) features.

118. The method of embodiment 116 or embodiment 117, wherein the prostate cancer is a prostate adenocarcinoma that does not comprise small cell carcinoma, carcinoid tumor, mixed NE carcinoma, or large cell NE carcinoma.

119. The method of any one of embodiments 1-118, wherein the subject has received prior treatment for metastatic castration resistant prostate cancer (mCRPC) with at least one androgen receptor (AR)-targeted therapy (for example treatment with abiraterone acetate, apalutamide, enzalutamide, or darolutamide), or at least one chemotherapy (for example docetaxel).

120. The method of any one of embodiments 1-119, wherein the prostate cancer is mCRPC that is metastatic to bone and/or lymph node, without evidence of metastasis to visceral organs.

121. The method of embodiment 120, wherein the subject has no known prior history of CNS involvement in prostate cancer.

122. The method of embodiment 120 or embodiment 121, wherein the subject has a PSA level of at least 2 ng/ml prior to treatment.

123. The method of any one of embodiments 120-122, wherein the subject has received prior treatment for mCRPC with at least one androgen receptor (AR)-targeted therapy (for example treatment with abiraterone acetate, apalutamide, enzalutamide, or darolutamide).

124. The method of any one of embodiments 120-123, wherein the subject has received no more than two prior taxane therapies.

125. The method of any one of embodiments 120-124, wherein the subject has not received prior cytotoxic chemotherapy (for example, cisplatin, carboplatin or etoposide).

126. The method of any one of embodiments 120-123 or 125, wherein the subject has not previously received chemotherapy.

127. The method of any one of embodiments 120-123, wherein the subject has received prior treatment for mCRPC with at least one androgen receptor (AR)-targeted therapy (for example treatment with abiraterone acetate, apalutamide, enzalutamide, or darolutamide).

128. The method of any one of embodiments 120-123, wherein the subject has received prior treatment for mCRPC with at least one taxane regimen.

129. The method of any one of embodiments 120-123, wherein the subject has received prior treatment for mCRPC with at least two taxane regimens.

130. The method of any one of embodiments 120-123, wherein the subject has received prior treatment for mCRPC with two taxane regimens.

131. The method of any one of embodiments 120-123, wherein the subject has received prior treatment for mCRPC with at least one dose of PSMA-targeted lutetium radioligand therapy (for example, lutetium Lu-177 vipivotide tetraxetan).

132. The method of any one of embodiments 120-123, wherein the subject has received prior treatment for mCRPC with a polyadenosine diphosphate-ribose polymerase inhibitors (PARPi).

133. The method of embodiment 120, wherein the subject has received
(a) prior treatment for mCRPC with at least one androgen receptor (AR)-targeted therapy (for example treatment with abiraterone acetate, apalutamide, enzalutamide, or darolutamide) and
(b) prior treatment for mCRPC with at least one taxane regimen,
   for example, the subject has received prior treatment for mCRPC with at least one androgen receptor (AR)-targeted therapy (for example treatment with abiraterone acetate, apalutamide, enzalutamide, or darolutamide) and prior treatment for mCRPC with two taxane regimens.

134. The method of embodiment 120, wherein the subject has received
(a) prior treatment for mCRPC with at least one androgen receptor (AR)-targeted therapy (for example treatment with abiraterone acetate, apalutamide, enzalutamide, or darolutamide),
(b) prior treatment for mCRPC with at least one taxane regimen, and
(c) prior treatment for mCRPC with at least one PARPi;
   for example the subject has received prior treatment for mCRPC with at least one androgen receptor (AR)-targeted therapy (for example treatment with abiraterone acetate, apalutamide, enzalutamide, or darolutamide), prior treatment for mCRPC with two taxane regimens, and prior treatment for mCRPC with a PARPi.

135. The method of any one of embodiments 132-134, wherein the participant has a known germline or somatic BRCA mutation.

136. The method of any one of embodiments 127-135, wherein the serum Prostate-Specific Antigen (PSA) level of the subject measured once the prior treatment with the androgen receptor (AR)-targeted therapy, the taxane therapy, the PSMA-targeted lutetium radioligand therapy or the PARPi has been completed, is not decreased compared to the PSA level measured before the administration of the androgen receptor (AR)-targeted therapy, the taxane therapy, the PSMA-targeted lutetium radioligand therapy or the PARPi, respectively.

137. The method of any one of embodiments 1-118, wherein the subject has prostate cancer that is metastatic to visceral tissue.

138. The method of embodiment 137, wherein the subject has no known prior history of CNS involvement in prostate cancer.

139. The method of embodiment 137 or embodiment 138, wherein the subject has a PSA level of at least 10 ng/ml prior to treatment.

140. The method of any one of embodiments 137-139, wherein the subject has received prior treatment with at least one androgen receptor (AR)-targeted therapy (for example treatment with abiraterone acetate, apalutamide, enzalutamide, or darolutamide).

141. The method of any one of embodiments 137-140, wherein the subject has received no more than two prior taxane therapies.

142. The method of any one of embodiments 137-141, wherein the subject has not received prior cytotoxic chemotherapy (for example, cisplatin, carboplatin or etoposide).

143. The method of any one of embodiments 1-118, wherein the subject has oligometastatic metastatic castration-sensitive prostate cancer (mCSPC).

144. The method of embodiment 143, wherein the subject has recurrent disease after definitive treatment to prostate, wherein the prostate cancer is metastatic to bone and/or lymph node, without evidence of metastasis to visceral organs.

145. The method of embodiment 142 or embodiment 144, wherein the subject has no more than five total lesions on imaging, for example up to five lesions PSMA-PET or no more than three lesions by conventional imaging.

146. The method of any one of embodiments 142-145, wherein the subject has not received an ADT or ARPI within one year of administration of the anti-hK2/anti-CD3 bispecific antibody or a bispecific fragment thereof.

147. The method of any one of embodiments 1-146, wherein the subject has non-castrate levels of testosterone of greater than 150 ng/mL.

148. The method of any one of embodiments 1-143, wherein the subject has:
(a) received orchiectomy or medical castration; or
(b) has not undergone orchiectomy and is receiving ongoing androgen deprivation therapy with a gonadotropin releasing hormone (GnRH) analog (agonist or antagonist) prior to being administered with the isolated anti-hK2/anti-CD3 bispecific antibody or a bispecific fragment thereof.

149. The method of any one of embodiments 1-143 or embodiment 148, wherein concurrent use of any other anticancer treatment (including non-palliative radiotherapy) or investigational agent, or prior anticancer treatments (including non-palliative radiotherapy) is discontinued for at least two weeks prior to the subject being administered with the isolated anti-hK2/anti-CD3 bispecific antibody or a bispecific fragment thereof, with the exception that a prior GnRH analog therapy according to embodiment 137(b) may be continued based on the physician's assessment.

150. The method of any one of embodiments 1-149, wherein the subject has one or more of the following:
(a) Eastern Cooperative Oncology Group (ECOG) performance status of 0 or 1;
(b) Hematology laboratory parameters within the following ranges, independent of transfusion within 7 days, or growth factors administered within three weeks prior to the subject being administered with the isolated anti-hK2/anti-CD3 bispecific antibody or a bispecific fragment thereof:
   a. Hemoglobin ≥9.0 g/dL
   b. Absolute neutrophil count ≥1.5×10⁹/L
   c. Platelet count ≥75×109/L; and/or
(c) Clinical chemistry laboratory parameters within the following range prior to the subject being administered with the isolated anti-hK2/anti-CD3 bispecific antibody or a bispecific fragment thereof:
   (i) Alanine aminotransferase (ALT) and aspartate aminotransferase (AST)≤2.5×ULN (or ≤4×ULN if the subject has tumor involvement in the liver)
   (ii) Serum albumin ≥3.0 g/dL
   (iii) Serum total bilirubin ≤1.5×ULN (or ≤1.5×ULN conjugated bilirubin if the subject has congenital bilirubinemia, such as Gilbert's syndrome)
   (iv) Estimated or measured glomerular filtration rate (GFR)≥50 mL/min/1.73 m2

151. The method of any one of embodiments 1-150, wherein the method provides a decrease in the serum Prostate-Specific Antigen (PSA) level of the subject, wherein the decrease is relative to the PSA level of the subject prior to the administration of the anti-hK2/anti-CD3 bispecific antibody or a bispecific fragment thereof.

152. The method of embodiment 151, wherein the PSA level decreases by 50% or more compared to the PSA level of the subject prior to the administration of the anti-hK2/anti-CD3 bispecific antibody or a bispecific fragment thereof.

153. The method of any one of embodiments 1-152, wherein the method provides for no disease progression in the subject, wherein disease progression is assessed according to the Prostate Cancer Working Group 3 (PCWG3) Criteria (Scher et al. 2016 Journal of Clinical Oncology 34(12): 1402-1418)).

154. The method of any one of embodiments 1-153, wherein the method provides a Partial Response or better according to RECIST version 1.1 response criteria (Eisenhauer et al. 2009 European Journal of Cancer 45: 228-247) without evidence of bone progression according to PCWG3 in the subject.

155. The method of any one of embodiments 1-154, wherein no Adverse Event of Grade 3 or higher (according to the National Cancer Institute Common Terminology Criteria for Adverse Events (NCI-CTCAE) version 5.0) is observed in the subject.

156. The method of any one of embodiments 1-154, wherein Adverse Events of Grade 3 or higher (according to the National Cancer Institute Common Terminology Criteria for Adverse Events (NCI-CTCAE) version 5.0) are observed in 20% or fewer of subjects in a cohort of at least 20 subjects, for example 15% or fewer of subjects in a cohort of at least 20 subjects.

157. The method of any one of embodiments 1-156, wherein no event of Injection Site Reaction (ISR) of Grade 3 or higher (according to the National Cancer Institute Common Terminology Criteria for Adverse Events (NCI-CTCAE) version 5.0) is observed in the subject.

158. The method of any one of embodiments 1-156, wherein events of Injection Site Reactions (ISR) (according to the National Cancer Institute Common Terminology Criteria for Adverse Events (NCI-CTCAE) version 5.0) are observed in 10% or fewer subjects in a cohort of at least 20 subjects, for example 5% or fewer of subjects in a cohort of at least 20 subjects.

159. The method of any one of embodiments 1-158, wherein no event of Fatigue of Grade 3 or higher (according to the National Cancer Institute Common Terminology Criteria for Adverse Events (NCI-CTCAE) version 5.0) is observed in the subject.

160. The method of any one of embodiments 1-154 or 156-158, wherein events of Fatigue (according to the National Cancer Institute Common Terminology Criteria for Adverse Events (NCI-CTCAE) version 5.0) are observed in 30% or fewer of subjects in a cohort of at least 20 subjects, for example 20% or fewer of subjects in a cohort of at least 20 subjects.

161. The method of any one of embodiments 1-160, wherein no event of Nausea of Grade 2 or higher (according to the National Cancer Institute Common Terminology Criteria for Adverse Events (NCI-CTCAE) version 5.0) is observed in the subject.

162. The method of any one of embodiments 1-154 or 156-160, wherein events of Nausea (according to the National Cancer Institute Common Terminology Criteria for Adverse Events (NCI-CTCAE) version 5.0) are observed in 20% or fewer of subjects in a cohort of at least 20 subjects, for example 15% or fewer of subjects in a cohort of at least 20 subjects.

163. The method of any one of embodiments 1-162, wherein no events of Anemia of Grade 3 or higher (according to the National Cancer Institute Common Terminology Criteria for Adverse Events (NCI-CTCAE) version 5.0) is observed in the subject.

164. The method of any one of embodiments 1-154 or 156-162, wherein events of Anemia (according to the National Cancer Institute Common Terminology Criteria for Adverse Events (NCI-CTCAE) version 5.0) are observed in 20% or fewer of subjects in a cohort of at least 20 subjects, for example 10% or fewer of subjects in a cohort of at least 20 subjects.

165. The method of any one of embodiments 1-164, wherein no event of Cytokine Release Syndrome (CRS) (according to the American Society for Transplantation and Cellular Therapy (ASTCT) guidelines) is observed in the subject.

166. The method of any one of embodiments 1-164, wherein no event of Cytokine Release Syndrome (CRS) of Grade 3 or higher (according to the American Society for Transplantation and Cellular Therapy (ASTCT) guidelines) is observed in the subject.

167. The method of any one of embodiments 1-164, wherein events of Cytokine Release Syndrome (CRS) (according to the American Society for Transplantation and Cellular Therapy (ASTCT) guidelines) are observed in 15% or fewer of subjects in a cohort of at least 20 subjects, for example 10% or fewer of subjects in a cohort of at least 20 subjects.

168. The method of any one of embodiments 1-164, wherein events of Cytokine Release Syndrome (CRS) of Grade 3 or higher (according to the American Society for Transplantation and Cellular Therapy (ASTCT) guidelines) are observed in 15% or fewer of subjects in a cohort of at least 20 subjects, for example 10% or fewer of subjects in a cohort of at least 20 subjects.

169. The method of any one of embodiments 1-164, wherein events of Cytokine Release Syndrome (CRS) of Grade 2 or higher (according to the American Society for Transplantation and Cellular Therapy (ASTCT) guidelines) are observed in 15% or fewer of subjects in a cohort of at least 20 subjects, for example 10% or fewer of subjects in a cohort of at least 20 subjects.

170. The method of any one of embodiments 1-169, wherein events of constipation (according to the National Cancer Institute Common Terminology Criteria for Adverse Events (NCI-CTCAE) version 5.0) are observed in 15% or fewer subjects in a cohort of at least 20 subjects.

171. The method of any one of embodiments 1-170, wherein events of back pain (according to the National Cancer Institute Common Terminology Criteria for Adverse Events (NCI-CTCAE) version 5.0) are observed in 20% or fewer subjects in a cohort of at least 20 subjects, for example in 15% or fewer subjects in a cohort of at least 20 subjects.

172. The method of any one of embodiments 1-171, wherein events of arthralgia (according to the National Cancer Institute Common Terminology Criteria for Adverse Events (NCI-CTCAE) version 5.0) are observed in 15% or fewer subjects in a cohort of at least 20 subjects, for example in 10% or fewer subjects in a cohort of at least 20 subjects.

173. The method of any one of embodiments 1-172, wherein no event of pyrexia (fever) of Grade 2 or higher (according to the National Cancer Institute Common Terminology Criteria for Adverse Events (NCI-CTCAE) version 5.0) is observed in the subject.

174. The method of any one of embodiments 1-173, wherein the method does not induce a Dose Limiting Toxicity (DLT) in the subject.

175. The method of any one of embodiments 1-174, wherein the subject does not have one or more or any of the following:
(a) active CNS involvement, except treated, stable and asymptomatic brain metastases requiring <10 mg prednisone or equivalent within two weeks prior to the first dose of study treatment are permitted;
(b) toxicity related to prior anticancer therapy which has not returned to Grade ≤1 or baseline levels;
(c) prior treatment with KLK2-targeted therapy;
(d) received immunosuppressive doses of systemic medications, such as glucocorticoids (doses >10 mg/day prednisone or equivalent) within 3 days prior to administration of the anti-hK2/anti-CD3 bispecific antibody or a bispecific fragment thereof;
(e) received or plans to receive any live, attenuated vaccine within four weeks before the first dose of study treatment or within four weeks after the last dose of study drug;
(f) malignancy diagnosis other than the disease under study within 2 years prior to the first dose of study drug, except for squamous and basal cell carcinoma of the skin, non-muscle invasive bladder cancer, or any malignancy considered cured or has minimal risk of recurrence within 1 year of first dose of study drug;
(g) solid organ or bone marrow transplantation;
(h) venous thromboembolic events (e.g., pulmonary embolism) within 1 month prior to the first dose of study treatment; uncomplicated (Grade ≤2) deep vein thrombosis is not considered exclusionary;
(i) active autoimmune disease within the 12 months prior to signing consent that requires systemic immunosuppressive medications;
(j) active infection or condition that requires treatment with systemic antibiotics within 7 days prior to the first dose of the anti-hK2/anti-CD3 bispecific antibody or a bispecific fragment thereof;
(k) any of the following within 6 months prior to signature of informed consent:
   vii. Myocardial infarction;
   viii. Severe or unstable angina;
   ix. Clinically significant ventricular arrhythmias;
   x. Congestive heart failure (according to New York Heart Association [NYHA] class II to IV);
   xi. Transient ischemic attack;
   xii. Cerebrovascular accident;
(l) clinically significant pulmonary compromise, particularly a requirement for supplemental oxygen use (>2 L/min by nasal cannula) to maintain adequate oxygenation;
(m) active or chronic hepatitis B virus (HBV) or hepatitis C virus (HCV) infection according to a local laboratory range;
(n) known positive test result for human immunodeficiency virus (unless stable on antiretroviral therapy with undetectable viral load and CD4 count above 200 cells/mm³);
(o) known allergies, hypersensitivity, or intolerance to the anti-hK2/anti-CD3 bispecific antibody or a bispecific fragment thereof, or its excipients;
(p) any serious underlying medical conditions or other issue that would impair the ability of the participant to receive or tolerate the planned treatment at the investigational site, to understand the informed consent, or any condition for which, in the opinion of the investigator, participation would not be in the best interest of the participant (e.g. compromise the well-being) or that could prevent, limit, or confound the protocol-specified assessments; and/or
(q) major surgery (e.g. requiring general anesthesia) within two weeks before first dose of the anti-hK2/anti-CD3 bispecific antibody or a bispecific fragment thereof, or has not recovered from surgery.

176. The method of any one of embodiments 1-175, wherein the anti-hK2/anti-CD3 bispecific antibody or a bispecific fragment thereof, is administered as a monotherapy.

177. The method of any one of embodiments 1-176, wherein the method comprises administering to the subject one or more additional therapeutic agent.

178. The method of embodiment 177, wherein the one or more additional therapeutic agent is one or more additional anti-cancer therapies.

179. The method of embodiment 177 or embodiment 178, wherein the one or more additional therapeutic agent is one or more immune modulating agents.

180. The method of any one of embodiments 1-179, wherein the anti-hK2/anti-CD3 bispecific antibody or a bispecific fragment thereof, is not administered to the subject in combination with a PSMAxCD28 bispecific antibody, wherein the isolated anti-PSMA/anti-CD28 bispecific antibody comprises a binding domain that binds specifically to PSMA, and a binding domain that binds specifically to CD28, wherein the PSMA binding domain comprises a HCDR1, a HCDR2 and a HCDR3 of a heavy chain variable region of SEQ ID NO: 63, and a LCDR1, a LCDR2, and a LCDR3 of a light chain variable region of SEQ ID NO: 64, and the combination dose of the anti-PSMA/anti-CD28 bispecific antibody is 0.5-2000 mg per administration.

181. The method of any one of embodiments 1-180, wherein the method does not comprise administering an androgen receptor pathway inhibitor (ARPI) or an anti-cancer chemotherapeutic agent to the subject, such as taxane.

182. The method of any one of embodiments 1-181, wherein the method consists essentially of administering a therapeutically effective amount of an isolated anti-hK2/anti-CD3 bispecific antibody or a bispecific fragment thereof, to the subject by intravenous (IV) infusion and optionally one or more step-up doses.

183. The method of any one of embodiments 1-182, wherein the prostate cancer is metastatic prostate cancer.

184. The method of any one of embodiments 1-183, wherein the prostate cancer is metastatic castration resistant prostate cancer (mCRPC).

185. The method of any one of embodiments 1-184, wherein the subject is an outpatient.

186. An isolated anti-hK2/anti-CD3 bispecific antibody or a bispecific fragment thereof as defined in any one of the preceding embodiments, for use in the method of any one of the preceding embodiments.

187. The method of any one of embodiments 1-116 and 143, wherein (prior to the administering of a therapeutically effective amount of an isolated anti-hK2/anti-CD3 bispecific antibody or a bispecific fragment thereof) the prostate cancer is a metastatic hormone sensitive prostate cancer (mHSPC).

188. The method of embodiment 187, wherein the mHSPC is a mHSPC with no (evidence of) metastasis to visceral organs.

189. The method of embodiment 187 or 188, wherein the mHSPC is a mHSPC with metastasis in no more than five (distinct) locations in the patient's body, more particularly a mHSPC with no more than 5 isocenters (lesions within radiation field for ablative metastasis-directed radiotherapy), for example with no more than 5 cancerous lesions.

190. The method of any one of embodiments 187-189, wherein the mHSPC is a mHSPC with metastasis in no more than five (distinct) locations in the patient's body but with no (evidence of) metastasis to visceral organs.

191. The method of any one of embodiments 187-190, wherein the mHSPC is a mHSPC with non-castrate levels of testosterone (e.g., at study entry), for example with levels of testosterone >150 ng/dL (e.g., due to testosterone supplementation prior to the administering of a therapeutically effective amount of an isolated anti-hK2/anti-CD3 bispecific antibody or a bispecific fragment thereof).

192. The method of any one of embodiments 187-191, wherein the patient does not have undergone orchiectomy.

193. The method of any one of embodiments 187-192, which further comprises administering metastasis-directed radiotherapy (MDR) to the (mHSPC) patient.

194. The method of any one of the preceding embodiments, wherein the anti-hK2/anti-CD3 bispecific antibody is pasritamig.

195. The method of any one of the preceding embodiments, wherein the method comprises administering a therapeutically effective amount of the isolated anti-hK2/anti-CD3 bispecific antibody.

196. An isolated anti-hK2/anti-CD3 bispecific antibody or a bispecific fragment thereof as defined in any one of the preceding embodiments, for use in the method of any one of the preceding embodiments.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Sequence and Structure of KLK2xCD3 Bispecific Antibody

The KLK2xCD3 bispecific antibody (i.e. anti-hK2/anti-CD3 bispecific antibody), (described in WO2021019389) was made by Janssen Biotech, Inc. The KLK2xCD3 bispecific antibody comprises a first domain that binds hK2 (KL2B30 in the format of a Fab) and a second domain that binds CD3 (CD3W245 in format of an scFv). The amino acid sequences of the KLK2xCD3 bispecific antibody are shown in Table 1 and Table 2, respectively. CDR sequences are defined according to Kabat.

The hk2 binding domain is in Fab format. The CD3 binding domain is in scFv format. The scFv comprises a linker polypeptide (SEQ ID NO: 20; GGSEGKSSGSGSESKSTGGS) between the VL and the VH. The bispecific antibody is in an IgG1 format. The VH of the hk2 binding domain is linked to a IgG1 heavy chain constant region containing the following mutations: the Fc silencing mutations (L234A/L235A/D265S) and the T350V/T366L/K392L/T394W mutations designed to promote selective heterodimerization. The VL of the hk2 binding domain is linked to a IgG1 light chain constant region. The scFv is linked to an IgG1 heavy chain constant region containing the following mutations: the Fc silencing mutation (L234A/L235A/D265S) and the T350V/L351Y/F405A/Y407V mutations designed to promote selective heterodimerization. The bispecific antibody comprises a lysine (K477) at the C-terminus of both of the Fc domains (i.e. the HC1 and HC2 domains).

TABLE 1
Sequences of the hK2 binding domain of the KLK2xCD3 bispecific antibody

| | | SEQ ID |
|---|---|---|
| Region | Sequence | NO: |
| HCDR1 | SYYWS | 1 |
| HCDR2 | YIYYSGSTNYNPSLKS | 2 |
| HCDR3 | TTIFGVVTPNFYYGMDV | 3 |
| LCDR1 | RASQGISSYLA | 4 |
| LCDR2 | AASTLQS | 5 |
| LCDR3 | QQLNSYPLT | 6 |
| VH | QVQLQESGPGLVKPSETLSLTCTVSGGSISSYYWSWIRQ | 7 |
| | PPGKGLEWIGYIYYSGSTNYNPSLKSRVTISVDTSKNQFS | |
| | LKLSSVTAADTAVYYCAGTTIFGVVTPNFYYGMDVWG | |
| | QGTTVTVSS | |
| VL | DIQMTQSPSFLSASVGDRVTITCRASQGISSYLAWYQQKP | 8 |
| | GKAPKFLIYAASTLQSGVPSRFSGSGSGTEFTLTISSLQPE | |
| | DFATYYCQQLNSYPLTFGGGTKVEIK | |
| HC | ASTKGPSVFPLAPSSKSTSGGTAALGCLVKDYFPEPVTVS | 54 |
| constant | WNSGALTSGVHTFPAVLQSSGLYSLSSVVTVPSSSLGTQ | |
| region | TYICNVNHKPSNTKVDKKVEPKSCDKTHTCPPCPAPEAA | |
| | GGPSVFLFPPKPKDTLMISRTPEVTCVVVSVSHEDPEVKF | |
| | NWYVDGVEVHNAKTKPREEQYNSTYRVVSVLTVLHQD | |
| | WLNGKEYKCKVSNKALPAPIEKTISKAKGQPREPQVYVL | |
| | PPSREEMTKNQVSLLCLVKGFYPSDIAVEWESNGQPENN | |
| | YLTWPPVLDSDGSFFLYSKLTVDKSRWQQGNVFSCSVM | |
| | HEALHNHYTQKSLSLSPG | |
| LC | RTVAAPSVFIFPPSDEQLKSGTASVVCLLNNFYPREAKVQ | 56 |
| constant | WKVDNALQSGNSQESVTEQDSKDSTYSLSSTLTLSKADY | |
| region | EKHKVYACEVTHQGLSSPVTKSFNRGEC | |
| HC | QVQLQESGPGLVKPSETLSLTCTVSGGSISSYYWSWIRQP | 9 |
| | PGKGLEWIGYIYYSGSTNYNPSLKSRVTISVDTSKNQFSL | |
| | KLSSVTAADTAVYYCAGTTIFGVVTPNFYYGMDVWGQ | |
| | GTTVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHTC | |
| | PPCPAPEAAGGPSVFLFPPKPKDTLMISRTPEVTCVVVSV | |
| | SHEDPEVKFNWYVDGVEVHNAKTKPREEQYNSTYRVVS | |
| | VLTVLHQDWLNGKEYKCKVSNKALPAPIEKTISKAKGQ | |
| | PREPQVYVLPPSREEMTKNQVSLLCLVKGFYPSDIAVEW | |
| | ESNGQPENNYLTWPPVLDSDGSFFLYSKLTVDKSRWQQ | |
| | GNVFSCSVMHEALHNHYTQKSLSLSPGK | |
| LC | DIQMTQSPSFLSASVGDRVTITCRASQGISSYLAWYQQKP | 10 |
| | GKAPKFLIYAASTLQSGVPSRFSGSGSGTEFTLTISSLQPE | |
| | DFATYYCQQLNSYPLTFGGGTKVEIKRTVAAPSVFIFPPS | |
| | DEQLKSGTASVVCLLNNFYPREAKVQWKVDNALQSGNS | |
| | QESVTEQDSKDSTYSLSSTLTLSKADYEKHKVYACEVTH | |
| | QGLSSPVTKSFNRGEC | |

TABLE 2
Sequences of the CD3 binding domain of the KLK2xCD3 bispecific antibody

| | | SEQ ID |
|---|---|---|
| Region | Sequence | NO: |
| HCDR1 | RYNMN | 11 |
| HCDR2 | SISTSSNYIYYADSVKG | 12 |
| HCDR3 | GWGPFDY | 13 |
| LCDR1 | RARQSIGTAIH | 14 |
| LCDR2 | YASESIS | 15 |
| LCDR3 | QQSGSWPYT | 16 |
| VH | EVQLVESGGGLVKPGGSLRLSCAASGFTFSRYNMNWVR | 17 |
| | QAPGKGLEWVSSISTSSNYIYYADSVKGRFTFSRDNAKN | |
| | SLDLQMSGLRAEDTAIYYCTRGWGPFDYWGQGTLVTVS | |
| | S | |
| VL | DIQMTQSPSSLSASVGDRVTITCRARQSIGTAIHWYQQKP | 18 |
| | GKAPKLLIKYASESISGVPSRFSGSGSGTDFTLTISSLQPED | |
| | FATYYCQQSGSWPYTFGQGTKLEIK | |
| scFv linker | GGSEGKSSGSGSESKSTGGS | 20 |
| scFv | DIQMTQSPSSLSASVGDRVTITCRARQSIGTAIHWYQQKP | 59 |
| | GKAPKLLIKYASESISGVPSRFSGSGSGTDFTLTISSLQPED | |
| | FATYYCQQSGSWPYTFGQGTKLEIKGGSEGKSSGSGSES | |
| | KSTGGSEVQLVESGGGLVKPGGSLRLSCAASGFTFSRYN | |
| | MNWVRQAPGKGLEWVSSISTSSNYIYYADSVKGRFTFSR | |
| | DNAKNSLDLQMSGLRAEDTAIYYCTRGWGPFDYWGQG | |
| | TLVTVSS | |
| HC | EPKSSDKTHTCPPCPAPEAAGGPSVFLFPPKPKDTLMISR | 55 |
| constant | TPEVTCVVVSVSHEDPEVKFNWYVDGVEVHNAKTKPRE | |
| region | EQYNSTYRVVSVLTVLHQDWLNGKEYKCKVSNKALPA | |
| | PIEKTISKAKGQPREPQVYVYPPSREEMTKNQVSLTCLVK | |
| | GFYPSDIAVEWESNGQPENNYKTTPPVLDSDGSFALVSK | |
| | LTVDKSRWQQGNVFSCSVMHEALHNHYTQKSLSLSPG | |
| HC | DIQMTQSPSSLSASVGDRVTITCRARQSIGTAIHWYQQKP | 19 |
| | GKAPKLLIKYASESISGVPSRFSGSGSGTDFTLTISSLQPED | |
| | FATYYCQQSGSWPYTFGQGTKLEIKGGSEGKSSGSGSES | |
| | KSTGGSEVQLVESGGGLVKPGGSLRLSCAASGFTFSRYN | |
| | MNWVRQAPGKGLEWVSSISTSSNYIYYADSVKGRFTFSR | |
| | DNAKNSLDLQMSGLRAEDTAIYYCTRGWGPFDYWGQG | |
| | TLVTVSSEPKSSDKTHTCPPCPAPEAAGGPSVFLFPPKPK | |
| | DTLMISRTPEVTCVVVSVSHEDPEVKFNWYVDGVEVHN | |
| | AKTKPREEQYNSTYRVVSVLTVLHQDWLNGKEYKCKV | |
| | SNKALPAPIEKTISKAKGQPREPQVYVYPPSREEMTKNQ | |
| | VSLTCLVKGFYPSDIAVEWESNGQPENNYKTTPPVLDSD | |
| | GSFALVSKLTVDKSRWQQGNVFSCSVMHEALHNHYTQ | |
| | KSLSLSPGK | |

### Example 2: A First-In-Human Study of KLK2xCD3 Bispecific Antibody in Participants with Advanced Prostate Cancer: Outline of the Study

A first-in-human (FIH) open-label, multicentre, Phase 1 study was conducted to evaluate the safety, pharmacokinetics, pharmacodynamics, and preliminary antitumor activity of KLK2xCD3 bispecific antibody administered to adult participants with advanced prostate cancer (PC) following at least 1 prior line of therapy (as defined in Example 3). The study is split into 2 parts: dose escalation (Part 1) and dose expansion (Part 2).

139 participants were dosed in Part 1 of the study (102 were administered the KLK2xCD3 antibody by SC administration and 37 by IV administration). It is estimated that 80 participants will be treated in Part 2.

During the study, safety was monitored at each dose escalation step and will be monitored at regular intervals during cohort expansion. Continuous reassessment for dose limiting toxicities (DLTs) was used to help minimize the potential risks associated with the study drug. Cumulative data from subsequent treatment cycles is monitored for late-onset toxicities.

### Example 3: Study Population

Screening for eligible participants was performed within 30 days before administration of the study drug. The inclusion and exclusion criteria for enrolling participants in this study are described below.

### Inclusion Criteria

Each potential participant must satisfy all of the following criteria to be enrolled in the study:

### Age

1. 18 years of age or older.

### Disease Characteristics

2. Advanced PC: Histologically confirmed adenocarcinoma of the prostate as defined by Prostate Cancer Working Group 3 (PCWG3). Adenocarcinoma with small cell or neuroendocrine (NE) features is permitted. However, small cell carcinoma, carcinoid tumor, mixed NE carcinoma, or large cell NE carcinoma is disallowed.
3. Part 1:
   1) Measurable or evaluable disease.
   2) Prior treatment for mCRPC with at least 1 prior novel AR-targeted therapy (ie, abiraterone acetate, apalutamide, enzalutamide, darolutamide), or chemotherapy (eg, docetaxel).
Part 2A: Bone or lymph node metastases
   1) Participant must have mCRPC that is metastatic either to bone, lymph node, or both without evidence of either measurable or non-measurable metastasis to visceral organs at the time of the screening scan and meet the Inclusion/Exclusion criteria.
   2) Must not have any known prior history of CNS involvement of prostate cancer.
   3) PSA must measure at least 2 ng/mL at screening.
   4) Must have received at least one line of novel AR-targeted therapy (ie, abiraterone acetate, apalutamide, enzalutamide, etc.).
   5) Must have received no more than 2 previous taxane regimens.
   6) Participant must not have received other prior cytotoxic chemotherapy (ie, cisplatin, carboplatin, etoposide, etc.).
Part 2B: Bone or lymph node metastases (chemotherapy naïve)
   1) Participant must have mCRPC that is metastatic either to bone, lymph node, or both without evidence of either measurable or non-measurable metastasis to visceral organs at the time of the screening scan and meet the Inclusion/Exclusion criteria.
   2) Must not have any known prior history of CNS involvement of prostate cancer.
   3) PSA must measure at least 2 ng/mL at screening.
   4) Must have received at least one line of novel AR-targeted therapy (ie, abiraterone acetate, apalutamide, enzalutamide, etc.).
   5) Participant must not have received other prior cytotoxic chemotherapy (ie, docetaxel, cabazitaxel, carboplatin, etoposide, etc.).
Part 2C: Visceral metastases
   1) Participant must have either measurable or non-measurable cancerous lesions in visceral tissue with or without bone or lymph node metastases and meet the Inclusion/Exclusion criteria.
   2) Must not have any known prior history of CNS involvement of prostate cancer.
   3) PSA must measure at least 10 ng/mL at screening.
   4) Must have received at least one line of novel AR-targeted therapy (ie, abiraterone acetate, apalutamide, enzalutamide, etc.)
   5) Must have received no more than 2 previous taxane regimens.
   6) Participant must not have received other prior cytotoxic chemotherapy (ie, cisplatin, carboplatin, etoposide, etc.).
Part 2D: Oligometastatic mCSPC
   1) Participant must have recurrent disease after definitive treatment to prostate that is metastatic either to bone, lymph node, or both without evidence of either measurable or non-measurable metastasis to visceral organs.
   2) Participant must have no more than 5 total lesions on imaging:
      a) up to 5 lesions on PSMA-PET
      b) No more than 3 lesions by conventional imaging,
   3) Must not have received an ADT or ARPI≤12 months from initial dose
   4) Must have non-castrate levels of testosterone (>150 ng/mL)
5. Prior orchiectomy or medical castration; or, for participants who have not undergone orchiectomy, must be receiving ongoing androgen deprivation therapy with a gonadotropin-releasing hormone (GnRH) analog (agonist or antagonist) prior to the first dose of study drug and must continue this therapy throughout the treatment phase. (This criterion does not apply to Cohort 2D)
6. Concurrent use of any other anticancer treatment (including non-palliative radiotherapy) or investigational agent; prior anticancer treatments (including non-palliative radiotherapy) must be discontinued for at least two weeks before the first dose of study drug. (See separate criteria for Cohort 2D)
7. Eastern Cooperative Oncology Group (ECOG) performance status of 0 or 1.
8 Haematology laboratory parameters within the following ranges, independent of transfusion within 7 days, or growth factors within three weeks prior to first dose of study treatment:
   a. Haemoglobin ≥9.0 g/dL
   b. Absolute neutrophil count ≥1.5×10⁹/L
   c. Platelet count ≥75×10⁹/L
9. Clinical chemistry laboratory parameters within the following range before the first dose of study drug:
   a. Alanine aminotransferase (ALT) and aspartate aminotransferase (AST)≤2.5×ULN (or ≤4×ULN for participants with tumor involvement in the liver)
   b. Serum albumin ≥3.0 g/dL
   c. Serum total bilirubin 1.5×ULN; participants with congenital bilirubinemia, such as Gilbert's syndrome, may enrol if conjugated bilirubin is 1.5×ULN
   d. Estimated or measured glomerular filtration rate (GFR)≥50 mL/min/1.73 m²

### Sex and Contraceptive/Barrier Requirements

10. Must agree to all the following during the study and for 90 days after the last dose of study drug:
   Wear a condom when engaging in any activity that allows for passage of ejaculate to another person.
   Not to donate sperm or freeze for future use for the purpose of reproduction.
   Not plan to father a child.
In addition, a female of childbearing potential who is a partner of a male participant must use a highly effective method of contraception as a condom may break or leak.
Contraceptive (birth control) use should be consistent with local regulations regarding the acceptable methods of contraception for those participating in clinical studies. Typical use failure rates may differ from those when used consistently and correctly.

### Informed Consent

11. Must sign an informed consent form (ICF) indicating that he understands the purpose of, and procedures required for, the study and is willing to participate in the study.
12. Willing and able to adhere to the restrictions specified in this protocol.

### Exclusion Criteria

Any potential participant who meets any of the following criteria will be excluded from participating in the study:

### Disease Conditions

1. Active central nervous system (CNS) involvement. Exception: Treated, stable and asymptomatic brain metastases requiring <10 mg prednisone or equivalent within two weeks prior to the first dose of study drug are permitted.
2. Toxicity related to prior anticancer therapy have not returned to Grade ≤1 or baseline levels (except for alopecia, vitiligo, Grade 2 peripheral neuropathy, and radiation tissue fibrosis).

### Prior/Concomitant Therapy

3. Prior treatment with KLK2-targeted therapy.
4. Received immunosuppressive doses of systemic medications, such as glucocorticoids (doses >10 mg/day prednisone or equivalent) within 3 days prior to the first dose of study drug. A single course of glucocorticoids is permitted as prophylaxis for imaging contrast (ie, for participants with allergies to contrast). If glucocorticoids were used to treat immune-related adverse events associated with prior therapy, ≥7 days must have elapsed since the last dose of corticosteroid.
5. Received or plans to receive any live, attenuated vaccine within four weeks before the first dose of study drug or within four weeks after the last dose of study drug. Live, attenuated influenza vaccines are permitted as late as 7 days before the study treatment. Non-live or non-replication-competent vaccines approved or authorized for emergency use (eg, COVID-19) by local health authorities are allowed.

### Prior/Concurrent Medical Conditions

6. Malignancy diagnosis other than the disease under study within 2 years prior to the first dose of study drug, except for squamous and basal cell carcinoma of the skin, non-muscle invasive bladder cancer, or any malignancy considered cured or has minimal risk of recurrence within 1 year of first dose of study drug (in the opinion of both the investigator and sponsor's medical monitor).
7. Solid organ or bone marrow transplantation.
8. Venous thromboembolic events (eg, pulmonary embolism) within 1 month prior to the first dose of study drug; uncomplicated (Grade ≤2) deep vein thrombosis is not considered exclusionary.
9. Active autoimmune disease within the 12 months prior to signing consent that requires systemic immunosuppressive medications (eg, chronic corticosteroid, methotrexate, or tacrolimus).
10. Active infection or condition that requires treatment with systemic antibiotics within 7 days prior to the first dose of study drug.
11. Any of the following within 6 months prior to signature of informed consent:
   a. Myocardial infarction
   b. Severe or unstable angina
   c. Clinically significant ventricular arrhythmias
   d. Congestive heart failure (New York Heart Association [NYHA] class II to IV)
   e. Transient ischemic attack
   f. Cerebrovascular accident
12. Clinically significant pulmonary compromise, particularly a requirement for supplemental oxygen use (>2 L/min by nasal cannula) to maintain adequate oxygenation.
13. Active or chronic hepatitis B virus (HBV) or hepatitis C virus (HCV) infection:
   Hepatitis C infection defined by a positive hepatitis C antibody. Participants who test positive for hepatitis C antibody are eligible if RNA viral load is undetectable (spontaneous recovery or after completing treatment for HCV infection).
14. Known positive test result for human immunodeficiency virus (unless stable on antiretroviral therapy with undetectable viral load and CD4 count above 200 cells/mm³).
15. Known allergies, hypersensitivity, or intolerance to KLK2xCD3 bispecific antibody or its excipients (refer to the IB).
16. Any serious underlying medical conditions or other issue that would impair the ability of the participant to receive or tolerate the planned treatment at the investigational site, to understand the informed consent, or any condition for which, in the opinion of the investigator, participation would not be in the best interest of the participant (eg, compromise the well-being) or that could prevent, limit, or confound the protocol-specified assessments.
17. Had major surgery (eg, requiring general anaesthesia) within two weeks before first dose of study drug or has not recovered from surgery. Must not have major surgery planned during the time the participant is receiving study drug. Participants with recent or planned surgical procedures utilizing only local anaesthesia may participate.

### Other Exclusions

18. Employee of the investigator or study site, with direct involvement in the proposed study or other studies under the direction of that investigator or study site, as well as family members of the employees or the investigator.
19. Treatment with any CD3-directed therapy within 90 days prior to first dose of study drug.

### Example 4: Determination of the FIH Starting Dose

### SC Route of Administration

The slow absorption profile of mAbs following SC administration was expected to facilitate slower and steady T-cell activation with manageable safety as observed in the preclinical settings for other molecules (Engelberts et al. EBioMedicine. 2020; 52:102625; and Staflin K, et al. JCI Insight. 2020; 5(7):e133757). Moreover, preliminary observations with other CD3-redirector programs that have been in Phase 1 (Chari A et al., 2020, Session 653, Abstract 290, 62^{nd} American Society of Hematology Annual; and Garfall al., 2020, Session 653, Abstract 180, 62^{nd} American Society of Hematology Annual Meeting) also indicated that SC administration provided a favourable safety profile in terms of CRS (cytokine release syndrome) following the first dose. Therefore, the dose escalation study started with SC administration.

### Determination of the MABEL (Minimum Anticipated Biological Effect Level)

The human prostate tumor cell line, VCaP, was used in the in vitro functional assays as it endogenously expresses surface KLK2 and is sensitive to T-cell mediated killing with KLK2xCD3 bispecific antibody. PBMCs from 5 healthy donors were used as a conservative approach. T-cell activation was assessed in the final in vitro assays used to determine MABEL concentrations as the results from in vitro pharmacology studies demonstrated T-cell activation to be the most sensitive readout with EC values consistently lower than those of cytotoxicity and cytokine release.

Well-defined E:T ratios at the tumor site (eg, bone metastasis) in advanced PC patients are currently unknown. However, T-cell infiltrates are expected to be at low levels within both the primary prostate tumor and bone metastatic lesions in mCRPC patients (Schumacher et al. Science. 2015; 348(6230):69-74). Thus, in the MABEL enabling assay, an E:T ratio of 1:5 was utilized as it is expected to mimic the tumor microenvironment more closely in advanced PC and be on the conservative side to set the starting dose. In addition, an E:T ratio of 1:5 was used to determine MABEL concentrations for an HLE-BiTE molecule for gastric cancer indication where one would also expect minimal T-cell infiltrating lymphocytes. Overall, the clinical starting dose from this approach was proven safe and tolerable in the gastric cancer patients in the ongoing clinical trial and Liao et al., 2020, Poster presented at the Eleventh American Conference on Pharmacometrics. Finally, preliminary evidence in the literature also suggests very low E:T ratios to be representative of solid tumor malignancies at baseline conditions (Betts et al. LP DART. AAPS J, 2019; 21(4):66).

Consistent and dose-dependent T-cell activation was observed with CD69 upregulation at 48 hours, and CD25 upregulation at 48 and 96 hours among the timepoints tested (24, 48, and 96 hours). The most sensitive readout was T-cell activation (CD69) at 48 hours; therefore, it was used to determine the MABEL concentration of 51 ng/mL (0.4 nM) (Table 3).

TABLE 3
Summary of Median Effective Concentrations & Ranges
for T-cell Activation (CD25% & CD69%) with KLK2 ×
CD3 Bispecific Antibody Using Healthy Donor PBMCs
(n = 5) & VCaP target cells at an E:T Ratio of 1:5

| | | |
|---|---|---|
| Time (hours) | EC₅₀ (μg/mL) | Range |

CD25% (Median EC value and Range)

| | | |
|---|---|---|
| 24 | 0.077 | (0.048, 0.124) |
| 48 | 0.166 | (0.128, 0.217) |
| 96 | 0.063 | (0.042, 0.094) |

CD69% (Median EC value and Range)

| | | |
|---|---|---|
| 24 | 0.069 | (0.053, 0.092) |
| 48 | 0.051 | (0.041, 0.064) |
| 96 | ND | ND |

Abbreviations:
EC₅₀ = 50% maximal effective concentration;
E:T = effector-to-target ratio;
ND = Not determinable

### Allometric Scaling

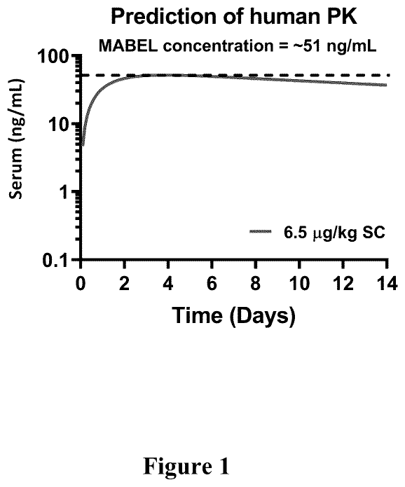

The human PK of KLK2xCD3 bispecific antibody was predicted from the cynomolgus monkey PK data using allometric scaling of clearance and volume of distribution based on body weight, while minipig PK data were used for human SC bioavailability estimations. Use of minipigs as an alternative predictive model to assess human SC bioavailability for antibodies have been well described (Richter et al., AAPS J. 2020; 22(3):63; and Zheng Y et al., MAbs. 2012; 4(2):243-255). Therefore, for KLK2xCD3 bispecific antibody, bioavailability results from minipigs (82%) were used along with the scaled absorption rate to estimate the dose that will result in a Cₘₐₓ equivalent to MABEL concentration following SC administration. Simulated PK profile in humans following SC administration are presented in FIG. 1.

A clinical starting dose of 6.5 μg/kg (500 μg with assumed median body weight of 80 kg for male patients with prostate cancer) administered via SC route was predicted to result in a Cₘₐₓ of 51 ng/mL (0.4 nM) which is the MABEL concentration as determined above. Based on an overall assessment of the in vitro and in vivo pharmacological data, and the MABEL-based FIH starting dose selection, a 500 μg dose of KLK2xCD3 bispecific antibody is estimated to result in drug exposures that have minimal biological activity in the participants treated in the FIH study.

The observed half-life (T1/2) of KLK2xCD3 bispecific antibody in monkeys was 11.5 days, and the allometrically scaled T1/2 based on body weight in human is estimated to be approximately 19.4 days. However, monoclonal antibodies may exhibit faster clearance at lower doses due to Target-Mediated Drug Disposition (TMDD). No TMDD was incorporated in the estimation of PK. Certain levels of TMDD may be expected in mCRPC patients, especially at lower doses of KLK2xCD3 bispecific antibody and with the SC route of administration. Hence, the Cmax prediction (for SC dosing) could be impacted by TMDD which may result in a lower than expected Cmax based on theoretical considerations. In such a situation, this remains a conservative approach where TMDD in humans is not considered in the MABEL calculation.

### Initial SC Administration Dose

To summarise, results from in vitro pharmacology studies using VCaP and PBMCs from 5 healthy donors were used to derive MABEL concentrations. The EC values for T-cell activation were consistently lower than that of cytotoxicity as well as cytokine release. Thus, as the most sensitive readout, T-cell activation was assessed in the final MABEL in vitro assays. An effector-to-target (E:T) cell ratio of 1:5 was used as it is expected to more closely mimic the tumor microenvironment in mCPRC and be on the conservative side to set the starting dose. The most sensitive read out, T-cell activation (CD69) at 48 hours was used to determine MABEL concentration of 51 ng/mL (0.4 nM) (Table 3). Human PK of KLK2xCD3 bispecific antibody following SC administration was predicted based on allometric scaling using cynomolgus monkey and Gottingen minipig PK results to derive the MABEL based FIH starting dose of 6.5 mg/kg.

The first-in-human (FIH) starting dose was set for 500 μg via once every three weeks (Q3W) SC administration, derived from an approximate 6.5 μg/kg dose with assumed median body weight of 80 kg for male patients with prostate cancer.

### Example 5: SC Administration Dose Escalation

After the starting dose, dose escalation was supported using an adaptive dose escalation strategy guided by the modified continual reassessment method (mCRM) based on a Bayesian Logistic Regression Model (BLRM) with overdose control. The mCRM design allows the use of all cumulative DLT data up to the current dose cohort.

To better understand the safety, tolerability, pharmacokinetics, pharmacodynamics, or preliminary antitumor activity, additional participants were enrolled for Cohorts 8 and 9 (referred to as PK/PD cohorts).

Step-up dose(s) were explored to mitigate the occurrence of cytokine-mediated toxicities, based on evaluation of the totality of clinical and supportive data. This involved the administration of one or more doses prior to a subsequent full treatment dose to mitigate the acute toxicities that may be associated with T cell activation and cytokine release. Each step-up dose was administered 3 or 7 days apart. The treatment dose was administered 7 days after the final step-up dose (except in cohorts 14 and 15 in which the treatment dose was administered 8 days and 11 days, respectively, after the final step-up dose).

The step-up dose can be administered on Day 1 and the treatment dose is then administered on Day 8. The first step-up dose can be administered on Day 1, the second step-up dose can be administered on Day 8 and the treatment dose can be administered on Day 15. The first step-up dose can be administered on Day 1, the second step-up dose can be administered on Day 4, the third step-up dose can be administered on Day 8 and the treatment dose can be administered on Day 11.

The study was initiated with once every three week (Q3W) treatment doses. Cohort 12 had once every week (Q1W) treatment doses for four weeks followed by Q3W treatment doses. Cohort 13 had Q1W treatment doses for seven weeks followed by Q3W treatment doses. Cohort 18 had once every six week treatment doses (Q6W).

Table 4 summarises the escalation of doses for different patient cohorts receiving SC administration of KLK2xCD3 bispecific antibody.

TABLE 4
Dose escalation of SC administration
of KLK2 × CD3 bispecific antibody

| Patient | Step-up | Step-up | Step-up | Treatment | Treatment | Patients |
|---|---|---|---|---|---|---|
| Cohort | dose 1* | dose 2* | dose 3* | dose (mg) | schedule | enrolled |
| 1 | — | — | — | 0.5 | Q3W | 2 |
| 2 | — | — | — | 1.5 | Q3W | 1 |
| 3 | — | — | — | 4.5 | Q3W | 4 |
| 4 | — | — | — | 13.5 | Q3W | 5 |
| 5 | — | — | — | 25 | Q3W | 6 |
| 6 | 15 | — | — | 50 | Q3W | 5 |
| 7 | 15 | — | — | 75 | Q3W | 2 |
| 8 | 10 | 50 | — | 100 | Q3W | 11 |
| 9 | 10 | 50 | — | 200 | Q3W | 12 |
| 10 | 10 | 50 | — | 400 | Q3W | 6 |
| 11 | 10 | 50 | — | 800 | Q3W | 13 |
| 12 | 10 | 50 | 200 | 800 | Q1W × 4 | 7 |
| | | | | | then Q3W | |
| 13 | 10 | 50 | 200 | 800 | Q1W × 7 | 6 |
| | | | | | then Q3W | |
| 14 | 10 | 50 | — | 400D8 | Q3W | 6 |
| 15 | 10 | 50 | 200 | 800D11 | Q3W | 5 |
| 17 | 10 | 50 | 200 | 2000 | Q3W | 5 |
| 18 | 10 | 50 | — | 200 | Q6W | 6 |

*each step-up dose is administered 3 or 7 days apart. Q3W = administered once every three weeks; Q6W = administered once every six weeks. Q1W × 4 = administered once every week for four weeks. Q1W × 7 = administered once every week for 7 weeks.

### Example 6: IV Administration

SC dosing started at 0.5 mg via once every three weeks SC administration. Doses were then escalated as outlined in Table 4. The safety of each dose was monitored. Doses trialled in Cohorts 1-11 were deemed safe. At this stage IV administration was considered.

Intravenous cohorts were distinct from subcutaneous cohorts. Participants receiving one route of administration (SC vs IV) were not eligible to switch to a different route of administration.

The initial treatment regimen for the IV route, consisting of step-up dose 1 (SU1) of 3.5 mg, step-up dose 2 (SU2) of 18 mg, and a treatment dose (TD) of 150 mg. In Cohort 11, subcutaneous dosing of 10 mg SU1, 50 mg SU2, and 800 mg TD administered every three weeks (Q3W) was evaluated and deemed tolerable with no observed DLTs. Additionally, in Cohort 5, a TD of 25 mg Q3W with no step-up dose was evaluated and deemed tolerable with no DLTs, representing the upper level of dosing for the initial exposure with IV administration. PK modelling and simulation demonstrates that the predicted Cₘₐₓ and AUCₜₐᵤ associated with the first treatment dose and the steady state of 150 mg IV would be approximately 2-fold lower than the Cₘₐₓ and AUCₜₐᵤ of the first treatment dose and the steady state of 800 mg SC administered every three weeks, respectively, providing an adequate safety margin.

KLK2xCD3 bispecific antibody pharmacokinetic data were collected from 56 participants with evaluable pharmacokinetic samples in Cohorts 1-11 where study drug was administered subcutaneously. An approximately dose proportional increase was observed in the mean Cmax and AUC values after the first treatment dose of KLK2xCD3 bispecific antibody. Modelling of the available KLK2xCD3 bispecific antibody pharmacokinetic data following SC administration, using a one-compartment model in Nonlinear mixed-effects modelling software (NONMEM®, Version 7.4.0; ICON plc, Hanover, MD, USA), as well as simulation to predict the exposure following SC administration were conducted. SC bioavailability was assumed to be 50% as a conservative approach to predict IV starting dose, which is in line with published values from conventional monoclonal antibodies (Richter et al., 2014; 42(11):1881-1889). Preliminary analysis suggests an estimated half-life of approximately 15 days. This model was used to predict the mean pharmacokinetic profile for the proposed initial IV dosing regimen. Predicted Cmax and AUCtau at each dosing level for both the step-up and the treatment dose is provided in Table 5.

TABLE 5
Predicted serum KLK2 × CD3 bispecific antibody
exposure metrics from IV initial regimen and SC Cohort 11

| | | | |
|---|---|---|---|
| Step-Up | Step-Up | First Treatment | Steady |
| Dose 1 | Dose 2 | Dose | State |

IV (3.5 mg D 0/18 mg D 7/150 mg D 14 and Q3W)

| | | | | |
|---|---|---|---|---|
| Cmax (ng/ml) | 634 | 3717 | 29837 | 43243 |
| AUCtau (ng · day/ml) | 3778 | 22154 | 398285 | 577249 |
| Ctrough (ng/ml) | 457 | 2678 | 11149 | 16159 |

SC (10 mg D 0/50 mg D 7/800 mg D 14 and Q3W)

| | | | | |
|---|---|---|---|---|
| Cmax (ng/ml) | 680 | 3954 | 57559 | 92174 |
| AUCtau (ng · day/ml) | 3692 | 22748 | 946069 | 1539581 |
| Ctrough (ng/ml) | 676 | 3907 | 32248 | 48828 |

D = day;
Q3W = every three weeks;
IV = intraveneous;
SC = subcutaneous;
Cmax = maximum serum concentration;
Ctrough = trough serum KLK2 × CD3 bispecific antibody concentration;
AUCtau = area under the serum KLK2 × CD3 bispecific antibody concentration-time curve during one dosing interval

The dose selection for the IV route accounts for the observed and predicted Cₘₐₓ and AUCₜₐᵤ for the step-up dosing regimen as well as the upper exposure range at the treatment dose. PK modelling and simulation demonstrate that the predicted Cₘₐₓ and AUCₜₐᵤ associated with the first SU1 dose of 3.5 mg IV and SU2 of 18 mg IV, would approximate the Cₘₐₓ and AUCₜₐᵤ of SU1 of 10 mg SC and SU2 of 50 mg SC, respectively. Additionally, the predicted Cₘₐₓ for a SU1 of 3.5 mg IV would be approximately 2.6-fold lower than the observed Cₘₐₓ of the first treatment dose at the 25 mg dose level which was administered subcutaneously and deemed to be safe without any step-up dosing in Cohort 5. PK modelling and simulation using a treatment dose of 150 mg IV shows similar Cₘₐₓ and approximately 80% of AUCₜₐᵤ as compared with a 400 mg Treatment Dose that has been studied in Cohort 10 and approximately 50% of Cₘₐₓ and 40% of AUCₜₐᵤ relative to the SC treatment dose of 800 mg studied in Cohort 11. Both cohorts were deemed to be safe. The selection of these doses provides an adequate safety margin to justify the initial treatment regimen for the IV route, consisting of SU1 of 3.5 mg, SU2 of 18 mg, and TD of 150 mg.

### Example 7: IV Administration Dose Escalation

IV dose escalation was also supported using an adaptive dose escalation strategy guided by the modified continual reassessment method (mCRM) based on a Bayesian Logistic Regression Model (BLRM) with overdose control. The mCRM design allows the use of all cumulative DLT data up to the current dose cohort.

Step-up dose strategies which involve administration of one or more doses prior to a subsequent full treatment dose were used to mitigate the acute toxicities that may be associated with T cell activation and cytokine release. Each step-up dose was administered 3 or 7 days apart. Each treatment dose was administered 7 days after step-up dose 2.

The first step-up dose can be administered on Day 1, the second step-up dose can be administered on Day 8 and the treatment dose can be administered on Day 15.

The IV dosing was escalated as shown in Table 6.

TABLE 6
Dose escalation of IV administration
of KLK2 × CD3 bispecific antibody

| Patient | Step-up | Step-up | Treatment | Treatment | Patients |
|---|---|---|---|---|---|
| Group | dose 1* | dose 2** | dose (mg) | schedule | enrolled |
| 16 | 3.5 | 18 | 150 | Q3W | 4 |
| 19 | 3.5 | 18 | 300 | Q3W | 12 |
| 20 | 3.5 | 18 | 300 | Q6W | 12 |
| 21 | 10 | 300 | 900 | Q6W | 3** |

*each step-up is administered 3 or 7 days apart;
**currently enrolling with planned N = 20 patients.

### Example 8: PK and Product Metabolism Results from the First-In-Human Study of KLK2xCD3 Bispecific Antibody in Participants with Advanced Prostate Cancer

### Single Dose SC or IV PK

PK data are available for participants in the ongoing FIH monotherapy study. Participants with at least one measurable serum concentration of KLK2xCD3 bispecific antibody post dose are included in the preliminary PK analysis.

Preliminary results after the first target dose of KLK2xCD3 bispecific antibody administered by SC or IV over a range of doses are presented below.

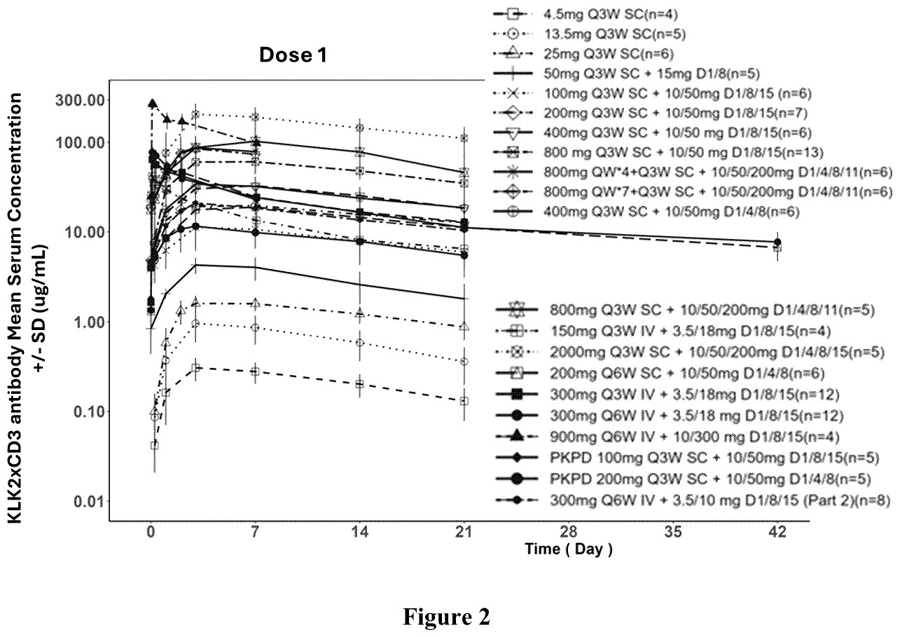

After the first SC administration of KLK2xCD3 bispecific antibody at target dose, the mean serum concentration of KLK2xCD3 bispecific antibody slowly increased at all dose levels, after which Tₘₐₓ occurred between 48 and 168 hours, followed by a slow elimination phase (FIG. 2) until the end of the 3- or 6-week dosing interval. Mean values for Cₘₐₓ and AUC_{0-21d} of KLK2xCD3 bispecific antibody increased approximately dose-proportionally with increasing doses (Table 7).

TABLE 7
Preliminary PK Results of KLK2 × CD3 bispecific antibody After Administration of
the First Treatment Dose (Dose 1) of KLK2 × CD3 bispecific antibody Q3W or Q6W SC Dosing.

| PK of | | | | | | | | AUC_{0-21 d}, | |
|---|---|---|---|---|---|---|---|---|---|
| KLK2 × CD3 | | | | | | Tₘₐₓ, h | | μg · h/mL ^{g} | |
| bispecific | | | | Cₘₐₓ, μg/mL | | Median | | Mean | |
| antibody | Cohort | | n | Mean (SD) | | (Range) | | (SD) | |
| First | C1 0.5 mg Q3W | | 2 | 0.0254 | | 168 | | 8.98 | |
| Treatment | C2 1.5 mg Q3W | | 1 | 0.058 | | 168 | | 17.7 | |
| Dose - SC | C3 4.5 mg Q3W | | 4 | 0.306 | (0.090) | 72 | (72-168) | 110 | (31.7) |
| | C4 13.5 mg Q3W | | 5 | 0.965 | (0.379) | 72 | (72-72) | 329 | (124) |
| | C5 25 mg Q3W | | 6 | 1.67 | (0.341) | 120 | (72-168) | 628 | (129) |
| | C6 50 mg Q3W (15 mg step-up) | | 5 | 4.35 | (0.899) | 72 | (48-168) | 1,524 | (463) |
| | C7 75 mg Q3W (15 mg step-up) | | 1 | 5.34 | | 168 | | NE | |
| | 100 mg Q3W (10/50 mg step-up) | C8 | 6 ^{a} | 11.6 | (4.26) | 120 | (72-168) | 4,526 | (1,684) |
| | | C8 | 5 | 12.3 | (4.78) | 72 | (48-168) | 4,269 | (1,189) |
| | | PKPD | | | | | | | |
| | 200 mg Q3W (10/50 mg step-up) | C9 | 7 ^{b} | 20.4 | (3.08) | 168 | (48-168) | 8,379 | (1,628) |
| | | C9 | 5 | 21.2 | (3.83) | 72 | (72-72) | 7,601 | (1,719) |
| | | PKPD | | | | | | | |

| C10 400 mg Q3W (10/50 mg step-up) | 6 ^{a} | 33.1 | (7.77) | 120 | (72-336) | 12,115 | (2,632) |
|---|---|---|---|---|---|---|---|
| C11 800 mg Q3W (10/50 mg step-up) | 12 ^{c} | 64.1 | (18.4) | 168 | (72-336) | 24,894 | (6,269) |
| C12 800 mg Q3W (10/50/200 mg step- | 6 | 93.4 | (25.4) | 168 | (48-168) | NE | |
| | up) | | | | | | |
| C13 800 mg (10/50/200 mg step-up) | 6 ^{d} | 86.6 | (27.5) | 120 | (48-168) | NE | |
| C14 400 mg Q3W (10/50 mg step-up) | 6 ^{a} | 33.0 | (9.34) | 120 | (72-168) | 13,024 | (4,006) |
| C15 800 mg Q3W (10/50/200 mg step- | 4 ^{e} | 94.1 | (37.8) | 168 | (72-168) | 40,795 | (13,467) |
| | up) | | | | | | |
| C17 2000 mg Q3W (10/50/200 mg step- | 5 ^{f} | 212 | (66.5) | 72 | (72-168) | 75,821 | (24,355) |
| | up) | | | | | | |
| C18 200 mg Q6W (10/50 mg step-up) | 6 ^{f} | 17.5 | (4.21) | 168 | (72-336) | 13,189 | (1,906) |

Key: AUC_{0-21 d} = area under the serum drug concentration-time curve from time 0 to 21 days post-dosing; C = Cohort; Cₘₐₓ = maximum serum KLK2 × CD3 bispecific antibody concentration; NE = not evaluable (due to insufficient data); PK = pharmacokinetics; SC = subcutaneous; SD = standard deviation; Tₘₐₓ = time to reach maximum concentration; Q3W = once every three weeks; Q6W = once every six weeks. Mean (SD) are presented if evaluable participants ≥3, and only mean values are presented if evaluable participants <3.
Participants with a missing concentration around the expected Cₘₐₓ were excluded from the descriptive statistics.
^{a} n = 5 for AUC_{0-21 d},
^{b} n = 6 for AUC_{0-21 d},
^{c} n = 11 for AUC_{0-21 d},
^{d} n = 5 for Cₘₐₓ,
^{e} n = 3 for AUC_{0-21 d},
^{f} n = 4 for AUC_{0-21 d},
^{g} AUC_{0-42 d}, μg · h/mL for C18
After the first IV administration of KLK2 × CD3 bispecific antibody at target dose, median Tₘₐₓ occurred at the end of infusion (2 hours) (Table 8).

TABLE 8
Preliminary PK Results of KLK2 × CD3 Bispecific Antibody
After Administration of the First Treatment Dose (Dose 1)
of KLK2 × CD3 Bispecific Antibody Q3W or Q6W IV Dosing

| PK of | | | | | | | | |
|---|---|---|---|---|---|---|---|---|
| KLK2 × CD3 | | | Cₘₐₓ, | | Tₘₐₓ, h | AUC_{0-21 d}, | | T_{1/2}, h |
| bispecific | | | μg/mL | | Median | μg · h/mL ^{c} | | Mean |
| antibody | Cohort | n | Mean (SD) | | (Range) | Mean (SD) | | (SD) |
| First | C16 IV 150 | 4 | 42.2 | (2.4) | 2 (2-8) | 6,910 | (1,721) | 289 (102) |
| Treatment | mg Q3W | | | | | | | |
| Dose - IV | (3.5/18 mg | | | | | | | |
| | step-up) | | | | | | | |
| | C19 IV 300 | 12 | 73.3 | (16.8) | 2 (2-4) | 12,811 | (3,460) | 365 (131) |
| | mg Q3W | | | | | | | |
| | (3.5/18 mg | | | | | | | |
| | step-up) | | | | | | | |
| | C20 IV 300 | 12 ^{a} | 78.1 | (11.0) | 2 (2-8) | 19,663 | (4,378) | 418 (168) |
| | mg Q6W | | | | | | | |
| | (3.5/18 mg | | | | | | | |
| | step-up) | | | | | | | |

| | | | | | | |
|---|---|---|---|---|---|---|
| C21 IV 900 | 4 ^{b} | 272 | (26.1) | 2 (2-2) | NE | NE |
| mg Q6W | | | | | | |
| (3.5/18 mg | | | | | | |
| step-up) | | | | | | |
| 2A IV 300 | 8 | 74 | (13.5) | 2 (2-4) | NE | NE |
| mg Q6W | | | | | | |
| (3.5/18 mg | | | | | | |
| step-up) | | | | | | |

Key: AUC_{0-21 d} = area under the serum drug concentration-time curve from time 0 to 21 days post-dosing; C = Cohort; Cₘₐₓ = maximum serum KLK2 × CD3 bispecific antibody concentration; IV = intravenous; NE = not evaluable (due to insufficient data); PK = pharmacokinetics; SD = standard deviation; t_{1/2} = half-life; Tₘₐₓ = time to reach maximum concentration; Q3W = once every three weeks; Q6W = once every six weeks.
Mean (SD) are presented if evaluable participants ≥3, and only mean values are presented if evaluable participants <3. Participants with a missing concentration around the expected Cₘₐₓ were excluded from the descriptive statistics.
^{a} n = 8 for AUC₀₋₄₂,
^{b} n = 3 for Cₘₐₓ and Tₘₐₓ,
^{c} AUC₀₋₄₂, for C20 and C21

Mean values for Cₘₐₓ and AUC_{0-21d} or AUC_{0-42d} of KLK2xCD3 bispecific antibody increased approximately dose-proportionally with increasing doses for the IV dose range that has been evaluated in this study. Mean T_{1/2} after IV administration was approximately 15 days.

Serum exposures of KLK2xCD3 bispecific antibody after IV dosing were compared with those observed after SC administration of KLK2xCD3 bispecific antibody:
The mean Cₘₐₓ observed in Cohort 16 (IV administration of 150 mg target dose (with 3.5 and 18 mg step-up doses) of KLK2xCD3 bispecific antibody) was 42.2 μg/mL which is comparable to that observed in Cohort 10 (SC administration of 400 mg target dose (with 10 and 50 mg step-up doses) of KLK2xCD3 bispecific antibody) which was 33.0 μg/mL. The mean AUC_{0-21d} of Cohort 16 was lower when compared with Cohort 10 (6,910 and 13,024 μg·h/mL, respectively).
The mean Cₘₐₓ observed in Cohort 19 (IV administration of 300 mg (with 3.5 and 18 mg step-up doses) of KLK2xCD3 bispecific antibody) of 73.3 to 78.1 μg/mL was comparable to that observed in Cohort 11 (800 mg Q3W SC administration (with 10 and 50 mg step-up doses) of KLK2xCD3 bispecific antibody) which was 64.1 μg/mL. The mean AUC_{0-21d} of Cohort 19 was lower when compared with Cohort 11 (12,811 and 24,894 μg h/mL, respectively).

Cohort 20 followed a similar administration to Cohort 19, except that instead of receiving the treatment dose Q3W they received the treatment dose Q6W. The Cₘₐₓ observed in Cohort 19 was 73.3 μg/mL. Similarly, the Cₘₐₓ observed in Cohort 20 was 78.1p g/mL.

### Multiple-Dose SC or IV PK

Following multiple SC administrations of KLK2xCD3 bispecific antibody, steady state was achieved following the fifth SC administration with Q3W dosing and achieved following the third SC administration with Q6W dosing. At steady state, serum C_{trough} increased approximately dose-proportionally following Q3W dosing (Table 9). The mean AR_{Ctrough} across SC cohorts ranged from 1.15 to 2.22 for Q3W dosing and was 1.05 for Q6W dosing (n=2).

TABLE 9
Preliminary PK Results of KLK2 × CD3 Bispecific
Antibody Following Dose 5 SC Injection with Q3W Dosing.

PK of
KLK2 × CD3

| bispecific | | | | C_{trough}, μg/mL | | AR_{Ctrough} |
|---|---|---|---|---|---|---|
| antibody | Cohort | | n | Mean (SD) | | Mean (SD) |
| Q3W or | C1 0.5 mg Q3W | | 1 | 0.0114 | | 1.63 |
| Q6W - SC | C4 13.5 mg Q3W | | 1 | 0.869 | | 1.64 |
| | C5 25 mg Q3W | | 5 | 2.11 | (0.961) | 2.22 (0.374) |
| | C6 50 mg Q3W (15 mg | | 2 | 4.14 | | 2 |
| | step-up) | | | | | |
| | 100 mg Q3W | C8 | 2 | 8.91 | | 1.15 |
| | (10/50 mg step-up) | PKPD | 2 | 6.03 | | 1.17 |
| | 200 mg Q3W | C9 | 5 | 22 | (3.71) | 1.62 (0.207) |
| | (10/50 mg step-up) | PKPD | 4 | 19.3 | (6.8) | 1.58 (0.511) |

| C10 400 mg Q3W (10/50 mg | 4 | 35.9 | (12.2) | 1.79 (0.279) |
|---|---|---|---|---|
| step-up) | | | | |
| C11 800 mg Q3W (10/50 mg | 7 | 57.4 | (20) | 1.61 (0.462) |
| step-up) | | | | |
| C14 400 mg Q3W (10/50 mg | 3 | 39.1 | (10.8) | 1.73 (0.131) |
| step-up) | | | | |
| C15 800 mg Q3W (10/50/200 | 3 | 110 | (13.8) | 1.31 (0.465) |
| step-up) | | | | |
| C17 2000 mg Q3W | 2 | 243 | | 1.81 |
| (10/50/200 mg step-up) | | | | |
| C18 200 mg Q6W (10/50 mg | 2 | 8.62 | | 1.05 |
| step-up) | | | | |

Key: AR_{Ctrough} = accumulation ratio for C_{trough}; C_{trough} = observed concentration just prior to the beginning of a dosing interval; C = Cohort; PK = pharmacokinetics; PKPD = pharmacokinetics/pharmacodynamics; SC = subcutaneous; SD = standard deviation; Q3W = once every three weeks; Q6W = once every six weeks. C_{trough} is the observed concentration at predose of Target Dose 5 for Q3W and Target Dose 3 for Q6W (C18). Mean (SD) are presented if evaluable participants ≥3, and only mean values are presented if evaluable participants <3.

Following multiple IV administrations of KLK2xCD3 bispecific antibody, steady state was similarly achieved following the fifth IV administration with Q3W dosing and achieved following the third IV administration with Q6W dosing. The mean ARCtrough across IV cohorts ranged from 1.76 to 1.98 for Q3W dosing based on the limited data (n=2 to 6) and was 1.21 for Q6W dosing (n=5) (Table 10).

TABLE 10
Preliminary PK Results of KLK2 × CD3 Bispecific
Antibody Following Dose 5 IV Administration with
Q3W Dosing and Dose 3 IV Administration with
Q6W Dosing (Ongoing Study 78278343PCR1001)

| PK | | | | |
|---|---|---|---|---|
| KLK2 × | | | C_{trough}, | |
| CD3 | | | μg/mL | AR_{Ctrough} |
| bispecific | | | Mean | Mean |
| antibody | Cohort | n | (SD) | (SD) |
| Q3W or | C16 IV 150 mg Q3W | 2 | 15.3 | 1.76 |
| Q6W - IV | (3.5/18 mg step-up) | | | |
| | C19 IV 300 mg Q3W | 6 | 30.5 (4.28) | 1.98 (0.305) |
| | (3.5/18 mg step-up) | | | |
| | C20 IV 300 mg Q6W | 5 | 10.3 (3.11) | 1.21 (0.0749) |
| | (3.5/18 mg step-up) | | | |

Key: AR_{Ctrough} = accumulation ratio for C_{trough}; C_{trough} = observed concentration just prior to the beginning of a dosing interval; C = Cohort; PK = pharmacokinetics; IV = intravenous; SD = standard deviation; Q3W = once every three weeks; Q6W = once every six weeks.
C_{trough} is the observed concentration at predose of a dosing interval.
C_{trough} is the observed concentration at predose of Target Dose 5 for Q3W.
Mean (SD) are presented if evaluable participants ≥3, and only mean values are presented if evaluable participants <3

### Example 9: Efficacy

Clinical activity was assessed using the following evaluations: computed tomography (CT) scan of the chest, abdomen, and pelvis with contrast as clinically indicated; magnetic resonance imaging (MRI) was substituted as clinically indicated (ie, for sites not adequately imaged using CT). Additional evaluations include serum prostate-specific antigen (PSA) and whole-body bone scans using technetium-99m (^{99m}Tc). Evaluation of treatment response will be performed according to Prostate Cancer Working Group 3 (PCWG3) criteria and Response Evaluation Criteria in Solid Tumors (RECIST) version 1.1 to evaluate progression of soft tissue lesions (CT or MRI).

Efficacy, measured as rates of PSA decrease of 50% or more (PSA50), has been observed at doses as low as 25 mg Q3W SC (Cohort 5). Any PSA50 rates from 17% to as high as 80% have been observed at active dose ranges across different cohorts, with evidence of improved disease control at higher exposures. Several participants across multiple active dose levels have achieved objective responses per RECIST 1.1 and/or remained on study for greater than a year with disease control, often with concurrent PSA responses.

### Example 10: Safety: Nature and Frequency of Adverse Events

All TEAEs (treatment emergent adverse events) were coded using MedDRA Version 26.1 and were summarized by SOC (system organ class) and PT (preferred term). Severity was graded according to National Cancer Institute Common Terminology Criteria for Adverse Events version 5.0 with the exception of CRS and ICANS, which were graded according to the American Society for Transplantation and Cellular Therapy guidelines. Relatedness of TEAEs to study treatment was assessed by the investigator.

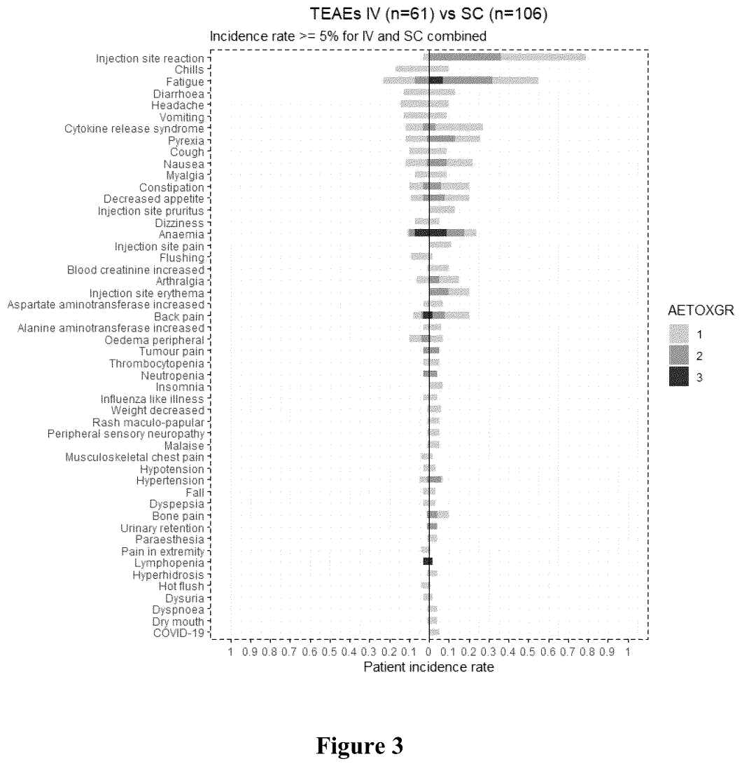

A summary of TEAEs reported in the First-in-Human monotherapy study of anti-hK2/anti-CD3 bispecific antibody in participants with advanced prostate cancer for the total. All Treated Analysis Set is presented in Table 11. TEAEs experienced by at least 10% of participants overall are presented by SOC and PT for the All Treated Analysis Set in Table 12. A summary of the TEAEs reported in the First-in-Human monotherapy study of anti-hK2/anti-CD3 bispecific antibody in participants with advanced prostate cancer is also shown in FIG. 3.

Overall, 159 participants have been treated: 102 participants received study drug via SC administration and 57 participants received study drug via IV administration. Table 11 shows a summary of the TEAE recorded for all the participants and a comparison between the percentage of TEAE in participants treated by SC or by IV. An AE occurred in 100% of SC participants whereas an AE occurred in only 78.9% of IV participants. These AEs were assessed by the investigator to determine whether they were related to the study agent (KLK2xCD3 bispecific antibody). A related AE was seen in 92.2% of SC participants whereas a related AE was seen in only 52.6% of IV participants. None of the IV participants had a related serious AE in comparison to 9.8% of SC participants who had a related serious AE. The percentage of SC participants who showed grade 3 or higher AE was higher than the percentage of IV participants who showed grade 3 or higher AE (48% and 8% respectively). The percentage of SC participants who showed related grade 3 or higher AE was also higher than the percentage of IV participants who showed related grade 3 or higher AE (10.8% and 3.5% respectively). These results show that treatment by IV results in less AE and related AE than administration of the treatment by SC.

TABLE 11
Overall Summary of TEAEs; All Treated Analysis Set

| | Total | | SC | | IV | |
|---|---|---|---|---|---|---|
| Analysis set: all treated | 159 | | 102 | | 57 | |
| Subjects with 1 or more treatment- | | | | | | |
| emergent: | | | | | | |
| AEs | 147 | (92.5%) | 102 | (100.0%) | 45 | (78.9%) |
| Related AEs ^{a} | 124 | (78.0%) | 94 | (92.2%) | 30 | (52.6%) |
| AEs leading to death ^{b} | 1 | (0.6%) | 1 | (1.0%) | 0 | |
| Related AEs leading to Death ^{a b} | 0 | | 0 | | 0 | |
| Serious AEs | 43 | (27.0%) | 40 | (39.2%) | 3 | (5.3%) |
| Related serious AEs ^{a} | 10 | (6.3%) | 10 | (9.8%) | 0 | |
| AEs leading to discontinuation of | 6 | (3.8%) | 6 | (5.9%) | 0 | |
| study drug | | | | | | |
| Related AEs leading to | 2 | (1.3%) | 2 | (2.0%) | 0 | |
| discontinuation of study drug ^{a} | | | | | | |
| Grade >=3 AEs | 57 | (35.8%) | 49 | (48.0%) | 8 | (14.0%) |
| Related Grade >=3 AEs ^{a} | 13 | (8.2%) | 11 | (10.8%) | 2 | (3.5%) |

| | | | | | |
|---|---|---|---|---|---|
| COVID-19 associated AEs ^{c} | 5 | (3.1%) | 5 | (4.9%) | 0 |
| COVID-19 associated serious AEs ^{c} | 1 | (0.6%) | 1 | (1.0%) | 0 |

Key:
AE = adverse event;
SC = subcutaneous;
IV = intravenous
^{a} An AE is assessed by the investigator as related to study agent.
^{b} AEs leading to death are based on AE outcome of Fatal.
^{c} COVID-19 associated AEs are based on events that code to a COVID-19 MedDRA term and events that are identified via the COVID-19 Case of AEs form.

Overall, the most frequently reported TEAEs were ISR (86 participants [54%]); fatigue (66 participants [41.5%]); CRS including symptoms of CRS (31 participants [19.5%]); and anaemia and nausea (29 participants each [18.2%]). For these most frequent TEAEs the percentage of participants reporting TEAEs was more in SC participants than IV participants:
ISR was observed in 83.3% of the SC cohort compared to only 1.8% of the IV cohort.
Fatigue was observed in 54.9% of the SC cohort compared to only 17.5% of the IV cohort.
Nausea was observed in 21.6% of the SC cohort compared to only 12.3% of the IV cohort.
Anaemia was observed in 23.5% of the SC cohort compared to only 8.8% of the IV cohort.
CRS was observed in 26.5% of the SC cohort compared to only 5.3% of the IC cohort.

TABLE 12
Number of Participants with TEAEs with Frequency of at Least 10% in
the Total Treatment Group by SOC and PT; All Treated Analysis Set

| | Total | SC | | IV | |
|---|---|---|---|---|---|
| Analysis set: all treated | 159 | 102 | | 57 | |
| Subjects with 1 or more | 147 (92.5%) | 102 | (100.0%) | 45 | (78.9%) |
| Treatment-Emergent AEs | | | | | |
| System Organ Class | | | | | |
| Preferred term | | | | | |
| General disorders and | 116 (73.0%) | 92 | (90.2%) | 24 | (42.1%) |
| administration site | | | | | |
| conditions | | | | | |
| Injection site reaction | 86 (54.1%) | 85 | (83.3%) | 1 | (1.8%) |
| Fatigue | 66 (41.5%) | 56 | (54.9%) | 10 | (17.5%) |
| Chills | 16 (10.1%) | 8 | (7.8%) | 8 | (14.0%) |
| Pyrexia | 16 (10.1%) | 11 | (10.8%) | 5 | (8.8%) |
| Gastrointestinal disorders | 76 (47.8%) | 53 | (52.0%) | 23 | (40.4%) |
| Nausea | 29 (18.2%) | 22 | (21.6%) | 7 | (12.3%) |
| Constipation | 25 (15.7%) | 19 | (18.6%) | 6 | (10.5%) |
| Diarrhoea | 23 (14.5%) | 16 | (15.7%) | 7 | (12.3%) |
| Vomiting | 19 (11.9%) | 13 | (12.7%) | 6 | (10.5%) |
| Musculoskeletal and | 68 (42.8%) | 52 | (51.0%) | 16 | (28.1%) |
| connective tissue disorders | | | | | |
| Back pain | 26 (16.4%) | 20 | (19.6%) | 6 | (10.5%) |
| Arthralgia | 19 (11.9%) | 16 | (15.7%) | 3 | (5.3%) |
| Metabolism and nutrition | 51 (32.1%) | 40 | (39.2%) | 11 | (19.3%) |
| disorders | | | | | |
| Decreased appetite | 25 (15.7%) | 19 | (18.6%) | 6 | (10.5%) |
| Nervous system disorders | 50 (31.4%) | 35 | (34.3%) | 15 | (26.3%) |
| Headache | 19 (11.9%) | 12 | (11.8%) | 7 | (12.3%) |
| Blood and lymphatic system | 40 (25.2%) | 35 | (34.3%) | 5 | (8.8%) |
| disorders | | | | | |
| Anaemia | 29 (18.2%) | 24 | (23.5%) | 5 | (8.8%) |
| Immune system disorders | 30 (18.9%) | 27 | (26.5%) | 3 | (5.3%) |
| Cytokine release | 30 (18.9%) | 27 | (26.5%) | 3 | (5.3%) |

syndrome

Key:
AE = adverse event;
SC = subcutaneous;
IV = intravenous
Note:
The output includes the reported syndromes of CRS, ISR, ICANS and sARR along with other AEs; however, AEs as the symptoms of CRS, ISR, ICANS or sARR are excluded.
Note:
Subjects are counted only once for any given event, regardless of the number of times they actually experienced the event.
Adverse events are coded using MedDRA Version 26.1.

TEAEs considered related to the study treatment by the investigator (TRAEs) that occurred in >5% of participants are presented by worst toxicity grade for SC and IV dosing cohorts in Table 13 and Table 14.

Overall, 94 participants (92.2%) and 30 participants (52.6%), respectively, reported at least one TRAE. The TRAEs experienced using different modes of administration differ substantially. The TRAEs most frequently reported with SC administration (n=102) were ISR (85 participants [83.3%]), fatigue (56 participants [54.90%]) and CRS (27 participants [26.5%]) (Table 13).

In the IV dosing cohorts (n=57), these TRAEs were not observed with the same level of frequency (ISR 1 participant [1.8%], fatigue 10 participants [17.5%]) and CRS 3 participants [5.3%]). 24 participants (23.5%) in the SC cohort experienced Grade 1 CRS compared to only 2 participants in the IV cohort (3.50%).

TABLE 13
Related TEAEs by Worst Toxicity Grade for SC Cohorts,
PT having Incidence of >5%; All Treated Analysis Set

| | Total | | Grade 1 | | Grade 2 | | Grade 3+ | |
|---|---|---|---|---|---|---|---|---|
| Analysis set: All | 102 | | | | | | | |
| Treated | | | | | | | | |
| Subjects with 1 or | 94 | (92.2%) | 36 | (35.3%) | 45 | (44.1%) | 13 | (12.7%) |
| more related AEs | | | | | | | | |
| Preferred term | | | | | | | | |
| Injection site | 85 | (83.3%) | 44 | (43.1%) | 38 | (37.3%) | 3 | (2.9%) |
| reaction | | | | | | | | |
| Fatigue | 34 | (33.3%) | 16 | (15.7%) | 13 | (12.7%) | 5 | (4.9%) |
| Cytokine release | 27 | (26.5%) | 24 | (23.5%) | 3 | (2.9%) | 0 | |
| syndrome | | | | | | | | |
| Pyrexia | 23 | (22.5%) | 10 | (9.8%) | 12 | (11.8%) | 1 | (1.0%) |

| | | | | | | | |
|---|---|---|---|---|---|---|---|
| Injection site | 21 | (20.6%) | 10 | (9.8%) | 10 | (9.8%) | 0 |
| erythema | | | | | | | |
| Injection site pain | 18 | (17.6%) | 13 | (12.7%) | 5 | (4.9%) | 0 |
| Injection site | 15 | (14.7%) | 13 | (12.7%) | 2 | (2.0%) | 0 |
| pruritus | | | | | | | |
| Chills | 9 | (8.8%) | 8 | (7.8%) | 1 | (1.0%) | 0 |
| Decreased | 9 | (8.8%) | 4 | (3.9%) | 5 | (4.9%) | 0 |
| appetite | | | | | | | |
| Headache | 7 | (6.9%) | 6 | (5.9%) | 1 | (1.0%) | 0 |
| Nausea | 7 | (6.9%) | 5 | (4.9%) | 2 | (2.0%) | 0 |

TABLE 14
Related TEAEs by Worst Toxicity Grade for IV Cohorts,
PT having Incidence of >5%; All Treated Analysis Set

| | Total | | Grade 1 | | Grade 2 | Grade 3+ |
|---|---|---|---|---|---|---|
| Analysis set: All | 57 | | | | | |
| Treated | | | | | | |
| Subjects with 1 or | 30 | (52.6%) | 18 | (31.6%) | 10 (17.5%) | 2 (3.5%) |
| more related AEs | | | | | | |
| Preferred term | | | | | | |
| Chills | 7 | (12.3%) | 5 | (8.8%) | 2 (3.5%) | 0 |
| Fatigue | 7 | (12.3%) | 5 | (8.8%) | 2 (3.5%) | 0 |
| Headache | 4 | (7.0%) | 4 | (7.0%) | 0 | 0 |
| Infusion related | 4 | (7.0%) | 2 | (3.5%) | 2 (3.5%) | 0 |
| reaction | | | | | | |
| Nausea | 4 | (7.0%) | 4 | (7.0%) | 0 | 0 |
| Alanine | 3 | (5.3%) | 1 | (1.8%) | 1 (1.8%) | 1 (1.8%) |
| aminotransferase | | | | | | |
| increased | | | | | | |
| Cytokine release | 3 | (5.3%) | 2 | (3.5%) | 1 (1.8%) | 0 |
| syndrome | | | | | | |
| Decreased | 3 | (5.3%) | 3 | (5.3%) | 0 | 0 |
| appetite | | | | | | |
| Pyrexia | 3 | (5.3%) | 3 | (5.3%) | 0 | 0 |

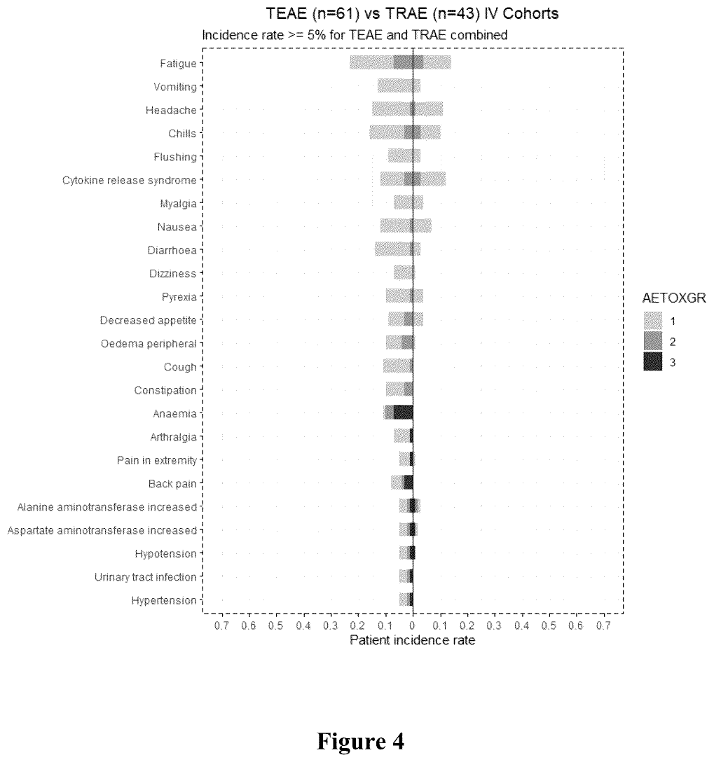

FIG. 3 compares the incidence rate of TEAE in SC cohorts compared to TEAE in IV cohorts. This demonstrates that the IV cohorts experienced significantly less TEAE and highlights IV administration as the preferred administration route due to the belier safety profile. FIG. 4 is a graphical representation which compares the frequency of TEAE and TRAE in IV cohorts. The figure shows the incidence rates that are greater than or equal to 50. This shows that the incidence rate of TEAE is low in the IV cohorts and the incidence rate of TRAE is even lower. The low incidence rate of TEAE in IV cohorts in comparison with the incidence rate of TEAE in SC cohorts illustrates advantageous properties of the IV route of administration.

Grade 3 or higher TEAEs were reported for 57 participants in the First-in-Human Study KLK2xCD3 bispecific antibody (Table 15). There were less Grade 3 or higher TEAEs reported for the IV cohort than the SC cohort; 49 participants from the SC cohort had Grade 3 or higher TEAEs compared to 8 participants from the IV cohort (48% and 14% respectively).

TABLE 15
Number of Participants with Treatment-emergent Grade
3 or Higher AEs by SOC and PT; All Treated Analysis
Set (Monotherapy Study 78278343PCR1001)

| | Total | SC | IV |
|---|---|---|---|
| Analysis set: all | 159 | 102 | 57 |
| treated | | | |
| Subjects with 1 or | 57 (35.8%) | 49 (48.0%) | 8 (14.0%) |
| more Grade 3 or | | | |
| Higher Treatment- | | | |
| Emergent AEs | | | |
| System Organ Class | | | |
| Preferred term | | | |
| Blood and lymphatic | 15 (9.4%) | 12 (11.8%) | 3 (5.3%) |
| system disorders | | | |
| Anaemia | 12 (7.5%) | 9 (8.8%) | 3 (5.3%) |
| Lymphopenia | 4 (2.5%) | 2 (2.0%) | 2 (3.5%) |
| Neutropenia | 1 (0.6%) | 1 (1.0%) | 0 |
| Musculoskeletal and | 13 (8.2%) | 10 (9.8%) | 3 (5.3%) |
| connective tissue | | | |
| disorders | | | |
| Back pain | 4 (2.5%) | 2 (2.0%) | 2 (3.5%) |
| Bone pain | 4 (2.5%) | 4 (3.9%) | 0 |
| Arthralgia | 2 (1.3%) | 1 (1.0%) | 1 (1.8%) |
| Musculoskeletal | 1 (0.6%) | 1 (1.0%) | 0 |
| chest pain | | | |
| Musculoskeletal pain | 1 (0.6%) | 1 (1.0%) | 0 |
| Neck pain | 1 (0.6%) | 1 (1.0%) | 0 |
| Pain in extremity | 1 (0.6%) | 1 (1.0%) | 0 |
| Pathological fracture | 1 (0.6%) | 1 (1.0%) | 0 |
| General disorders | 8 (5.0%) | 8 (7.8%) | 0 |
| and administration | | | |
| site conditions | | | |
| Fatigue | 6 (3.8%) | 6 (5.9%) | 0 |
| Injection site reaction | 3 (1.9%) | 3 (2.9%) | 0 |
| Pyrexia | 1 (0.6%) | 1 (1.0%) | 0 |
| Nervous system | 6 (3.8%) | 5 (4.9%) | 1 (1.8%) |
| disorders | | | |
| Ataxia | 1 (0.6%) | 1 (1.0%) | 0 |
| Dizziness | 1 (0.6%) | 1 (1.0%) | 0 |
| Facial paresis | 1 (0.6%) | 1 (1.0%) | 0 |
| Nervous system | 1 (0.6%) | 1 (1.0%) | 0 |
| disorder | | | |
| Spinal cord | 1 (0.6%) | 0 | 1 (1.8%) |
| compression | | | |
| Syncope | 1 (0.6%) | 1 (1.0%) | 0 |
| Gastrointestinal | 5 (3.1%) | 5 (4.9%) | 0 |
| disorders | | | |
| Abdominal pain | 1 (0.6%) | 1 (1.0%) | 0 |
| Diarrhoea | 1 (0.6%) | 1 (1.0%) | 0 |
| Gastric haemorrhage | 1 (0.6%) | 1 (1.0%) | 0 |
| Proctalgia | 1 (0.6%) | 1 (1.0%) | 0 |
| Vomiting | 1 (0.6%) | 1 (1.0%) | 0 |
| Infections and | 5 (3.1%) | 4 (3.9%) | 1 (1.8%) |
| infestations | | | |
| Urinary tract | 2 (1.3%) | 2 (2.0%) | 0 |
| infection | | | |
| Pneumonia | 1 (0.6%) | 0 | 1 (1.8%) |
| Rectal abscess | 1 (0.6%) | 1 (1.0%) | 0 |
| Sepsis | 1 (0.6%) | 1 (1.0%) | 0 |
| Spinal cord infection | 1 (0.6%) | 1 (1.0%) | 0 |
| Investigations | 5 (3.1%) | 4 (3.9%) | 1 (1.8%) |
| Aspartate | 3 (1.9%) | 2 (2.0%) | 1 (1.8%) |
| aminotransferase | | | |
| increased | | | |
| Gamma- | 3 (1.9%) | 3 (2.9%) | 0 |
| glutamyltransferase | | | |
| increased | | | |
| Alanine | 2 (1.3%) | 1 (1.0%) | 1 (1.8%) |
| aminotransferase | | | |
| increased | | | |
| Metabolism and | 5 (3.1%) | 4 (3.9%) | 1 (1.8%) |
| nutrition disorders | | | |
| Hyperglycaemia | 3 (1.9%) | 3 (2.9%) | 0 |
| Hypocalcaemia | 1 (0.6%) | 0 | 1 (1.8%) |
| Hyponatraemia | 1 (0.6%) | 1 (1.0%) | 0 |
| Vascular disorders | 5 (3.1%) | 4 (3.9%) | 1 (1.8%) |
| Hypertension | 3 (1.9%) | 2 (2.0%) | 1 (1.8%) |
| Embolism | 1 (0.6%) | 1 (1.0%) | 0 |
| Inferior vena cava | 1 (0.6%) | 1 (1.0%) | 0 |
| syndrome | | | |
| Neoplasms benign, | 4 (2.5%) | 4 (3.9%) | 0 |
| malignant and | | | |
| unspecified (incl | | | |
| cysts and polyps) | | | |
| Tumour pain | 3 (1.9%) | 3 (2.9%) | 0 |
| Metastases to liver | 1 (0.6%) | 1 (1.0%) | 0 |
| Renal and urinary | 4 (2.5%) | 2 (2.0%) | 2 (3.5%) |
| disorders | | | |
| Acute kidney injury | 2 (1.3%) | 1 (1.0%) | 1 (1.8%) |
| Hydronephrosis | 2 (1.3%) | 1 (1.0%) | 1 (1.8%) |
| Hepatobiliary | 3 (1.9%) | 1 (1.0%) | 2 (3.5%) |
| disorders | | | |
| Cholestasis | 1 (0.6%) | 0 | 1 (1.8%) |
| Hepatic pain | 1 (0.6%) | 0 | 1 (1.8%) |
| Hyperbilirubinaemia | 1 (0.6%) | 1 (1.0%) | 0 |
| Injury, poisoning and | 3 (1.9%) | 3 (2.9%) | 0 |
| procedural | | | |
| complications | | | |
| Fall | 2 (1.3%) | 2 (2.0%) | 0 |
| Humerus fracture | 1 (0.6%) | 1 (1.0%) | 0 |
| Subdural haematoma | 1 (0.6%) | 1 (1.0%) | 0 |
| Respiratory, thoracic | 3 (1.9%) | 3 (2.9%) | 0 |
| and mediastinal | | | |
| disorders | | | |
| Dysphonia | 1 (0.6%) | 1 (1.0%) | 0 |
| Dyspnoea | 1 (0.6%) | 1 (1.0%) | 0 |
| Pleural effusion | 1 (0.6%) | 1 (1.0%) | 0 |
| Pulmonary embolism | 1 (0.6%) | 1 (1.0%) | 0 |
| Cardiac disorders | 2 (1.3%) | 2 (2.0%) | 0 |
| Angina pectoris | 1 (0.6%) | 1 (1.0%) | 0 |
| Atrial fibrillation | 1 (0.6%) | 1 (1.0%) | 0 |

Key:
AE = adverse event;
SC = subcutaneous;
IV = intravenous
Note:
The output includes the reported syndromes of CRS, ISR, ICANS and sARR along with other AEs; however, AEs as the symptoms of CRS, ISR, ICANS or sARR are excluded.
Note:
Subjects are counted only once for any given event, regardless of the number of times they actually experienced the event.
Adverse events are coded using MedDRA Version 26.1.

Grade 3 or higher TEAEs considered related to treatment by the investigator were reported in 13 participants. There were less related Grade 3 or higher TEAEs reported for the IV cohort than the SC cohort; 11 participants from the SC cohort and 2 participants from the IV cohort (10.8% and 3.5% respectively) (Table 16).

TABLE 16
Number of Participants with Related Treatment-emergent
Grade 3 or Higher AEs by SOC and PT; All Treated Analysis
Set (Monotherapy Study 78278343PCR1001)

| | Total | SC | IV |
|---|---|---|---|
| Analysis set: all treated | 159 | 102 | 57 |
| Subjects with 1 or more | 13 (8.2%) | 11 (10.8%) | 2 (3.5%) |
| Related Treatment-Emergent | | | |
| Grade 3 or Higher AEs | | | |
| System Organ Class | | | |
| Preferred term | | | |
| General disorders and | 5 (3.1%) | 5 (4.9%) | 0 |
| administration site conditions | | | |
| Fatigue | 3 (1.9%) | 3 (2.9%) | 0 |
| Injection site reaction | 3 (1.9%) | 3 (2.9%) | 0 |
| Pyrexia | 1 (0.6%) | 1 (1.0%) | 0 |
| Blood and lymphatic system | 4 (2.5%) | 3 (2.9%) | 1 (1.8%) |
| disorders | | | |
| Lymphopenia | 2 (1.3%) | 1 (1.0%) | 1 (1.8%) |
| Anaemia | 1 (0.6%) | 1 (1.0%) | 0 |
| Neutropenia | 1 (0.6%) | 1 (1.0%) | 0 |
| Investigations | 2 (1.3%) | 1 (1.0%) | 1 (1.8%) |
| Alanine aminotransferase | 2 (1.3%) | 1 (1.0%) | 1 (1.8%) |
| increased | | | |
| Aspartate aminotransferase | 2 (1.3%) | 1 (1.0%) | 1 (1.8%) |
| increased | | | |
| Nervous system disorders | 2 (1.3%) | 2 (2.0%) | 0 |
| Ataxia | 1 (0.6%) | 1 (1.0%) | 0 |
| Syncope | 1 (0.6%) | 1 (1.0%) | 0 |
| Gastrointestinal disorders | 1 (0.6%) | 1 (1.0%) | 0 |
| Diarrhoea | 1 (0.6%) | 1 (1.0%) | 0 |
| Vascular disorders | 1 (0.6%) | 1 (1.0%) | 0 |
| Embolism | 1 (0.6%) | 1 (1.0%) | 0 |

Key:
AE = adverse event;
SC = subcutaneous;
IV = intravenous
Note:
The output includes the reported syndromes of CRS, ISR, ICANS and sARR along with other AEs; however, AEs as the symptoms of CRS, ISR, ICANS or sARR are excluded.
Note:
An AE is assessed by the investigator as related to study agent. Subjects are counted only once for any given event, regardless of the number of times they actually experienced the event
Adverse events are coded using MedDRA Version 26.1.

### Example 11: Safety: Cytokine Release Syndrome (CRS)

Overall, 31 participants (19.5%) experienced CRS including symptoms of CRS. SAEs of CRS were reported in 6 participants (3.8%). The number of CRS events experienced using different modes of administration differ substantially; 27 participants (26.5%) experienced CRS with SC administration, while only 4 participants (7.0%) experienced CRS with IV administration.

At the proposed RP2D) dose level of 300 mg IV, with step-up doses of 3.5 mg and 18 mg in Cohorts 19, 20 and expansion 2A, 1 (2.4%) of 42 participants experienced CRS including symptoms of CRS (Table 17). All events of CRS occurred at doses of 10 mg (SC or IV) or higher, with a mean time to onset of 2.0 days (range: 1 to 8) after the most recent exposure to KLK2 x CD3 antibody. The maximum grade reported for CRS was Grade 1 in 27 participants (17.0%) and Grade 2 in 4 participants (2.50%). Grade 3 or higher CRS has not been reported. Step-up dosing was implemented early in dose escalation and has mitigated the risk of severe CRS.

TABLE 17
Number of Participants with Treatment-emergent CRS
and Symptoms by PT; All Treated Analysis Set

| | | | | | IV | |
|---|---|---|---|---|---|---|
| | Total | | SC | | Total | RP2D |
| Analysis set: all | 159 | | 102 | | 57 | 42 |
| treated | | | | | | |
| Subjects with 1 or | 31 | (19.5%) | 27 | (26.5%) | 4 (7.0%) | 1 (2.4%) |
| more treatment- | | | | | | |
| emergent CRS or | | | | | | |
| Symptom of CRS | | | | | | |
| Cytokine Release | 30 | (18.9%) | 27 | (26.5%) | 3 (5.3%) | 0 |
| Syndrome | | | | | | |
| Symptom of | 21 | (13.2%) | 19 | (18.6%) | 2 (3.5%) | 1 (2.4%) |
| Cytokine Release | | | | | | |
| Syndrome | | | | | | |
| Pyrexia | 18 | (11.3%) | 17 | (16.7%) | 1 (1.8%) | 1 (2.4%) |
| Fatigue | 6 | (3.8%) | 6 | (5.9%) | 0 | 0 |
| Chills | 4 | (2.5%) | 3 | (2.9%) | 1 (1.8%) | 0 |
| Headache | 2 | (1.3%) | 2 | (2.0%) | 0 | 0 |
| Hypotension | 2 | (1.3%) | 2 | (2.0%) | 0 | 0 |
| Decreased appetite | 1 | (0.6%) | 1 | (1.0%) | 0 | 0 |
| Diarrhoea | 1 | (0.6%) | 1 | (1.0%) | 0 | 0 |
| Disease prodromal | 1 | (0.6%) | 1 | (1.0%) | 0 | 0 |
| stage | | | | | | |
| Hyperthermia | 1 | (0.6%) | 1 | (1.0%) | 0 | 0 |
| Hypoxia | 1 | (0.6%) | 1 | (1.0%) | 0 | 0 |
| Myalgia | 1 | (0.6%) | 1 | (1.0%) | 0 | 0 |

Key:
CRS = Cytokine Release Syndrome;
SC = subcutaneous;
IV = intravenous;
RP2D = recommended phase 2 dose
Note:
Subjects are counted only once for any given event, regardless of the number of times they actually experienced the event.
Note:
Participants in cohorts 19 (Q3W), 20 (Q6W) and expansion 2A (Q6W) received treatment at the putative RP2D dose levels of 3.5 mg, 18 mg, and 300 mg.
Adverse events are coded using MedDRA Version 26.1.

### Example 12: Safety: Injection Site Reactions (ISR)

Overall, ISR or symptoms of ISR were reported for 89 participants (56.00%). The number of ISR events experienced using different modes of administration differ substantially: 86 participants (84.3%) experienced ISR with SC administration, while only 3 participants (5.3) experienced ISR/infusion-site reaction with IV administration (Table 18). The occurrence of ISR with SC administration does not seem to be related to dose, volume of injection, or the dose concentration. If an ISR occurs when more than one injection per dose is administered, the ISR will tend to occur at all injection sites. The most frequently reported symptoms of ISR are listed in Table 18.

TABLE 18
Number of Participants with Treatment-emergent Local
Injection/ISR and Symptoms by PT; All Treated Analysis
Set (Monotherapy Study 78278343PCR1001)

| | Total | | SC | | IV |
|---|---|---|---|---|---|
| Analysis set: all | 159 | | 102 | | 57 |
| treated | | | | | |
| Subjects with 1 or | 89 | (56.0%) | 86 | (84.3%) | 3 (5.3%) |
| more treatment- | | | | | |
| emergent ISR or | | | | | |
| Symptom of ISR | | | | | |
| Injection/Infusion | 88 | (55.3%) | 85 | (83.3%) | 3 (5.3%) |
| Site Reaction ^{a} | | | | | |
| Symptom of Local | 40 | (25.2%) | 39 | (38.2%) | 1 (1.8%) |
| Injection/Infusion | | | | | |
| Site Reaction | | | | | |
| Injection site | 21 | (13.2%) | 21 | (20.6%) | 0 |
| erythema | | | | | |
| Injection site pain | 18 | (11.3%) | 18 | (17.6%) | 0 |
| Injection site pruritus | 15 | (9.4%) | 15 | (14.7%) | 0 |
| Chills | 2 | (1.3%) | 1 | (1.0%) | 1 (1.8%) |
| Injection site oedema | 2 | (1.3%) | 2 | (2.0%) | 0 |
| Injection site rash | 2 | (1.3%) | 2 | (2.0%) | 0 |
| Injection site | 2 | (1.3%) | 2 | (2.0%) | 0 |
| swelling | | | | | |
| Pruritus | 2 | (1.3%) | 2 | (2.0%) | 0 |
| Pyrexia | 2 | (1.3%) | 2 | (2.0%) | 0 |
| Administration site | 1 | (0.6%) | 1 | (1.0%) | 0 |
| pain | | | | | |
| Administration site | 1 | (0.6%) | 1 | (1.0%) | 0 |
| rash | | | | | |
| Affective disorder | 1 | (0.6%) | 1 | (1.0%) | 0 |
| Erythema | 1 | (0.6%) | 1 | (1.0%) | 0 |
| Injection site mass | 1 | (0.6%) | 1 | (1.0%) | 0 |
| Pain in extremity | 1 | (0.6%) | 1 | (1.0%) | 0 |
| Pneumonia | 1 | (0.6%) | 1 | (1.0%) | 0 |
| Rash maculo-papular | 1 | (0.6%) | 1 | (1.0%) | 0 |
| Skin induration | 1 | (0.6%) | 1 | (1.0%) | 0 |

Key:
ISR = Injection/Infusion Site Reaction;
SC = subcutaneous;
IV = intravenous
^{a} Including MedDRA Preferred Terms of injection site reaction and infusion site reaction.
Note:
Subjects are counted only once for any given event, regardless of the number of times they actually experienced the event.
Adverse events are coded using MedDRA Version 26.1.

### Example 13: Immunogenicity: Anti-Drug Antibody (ADA) Data

Preliminary results showed that 55 of 141 evaluable participants (39.0%) were positive for ADAs against the KLK2xCD3 bispecific antibody following either SC or IV administration of the KLK2xCD3 bispecific antibody. Of those, 49 of 102 evaluable participants (48.0%) developed ADA after receiving the KLK2xCD3 bispecific antibody via SC administration and 6 of 39 evaluable participants (15.4%) developed ADA after receiving the KLK2xCD3 bispecific antibody via IV administration. The ADAs appeared to have no impact on the safety or PK for these participants.

### Example 14: QSP Modelling to Guide RP2D Determination

Quantitative systems pharmacology (QSP) was also used to model suitable RP2D dosage regimens. The model takes into account different factors which can be used to model the clinical response to the KLK2xCD3 bispecific antibody.

In summary, the model has three elements:
a physiologically based pharmacokinetic (PBPK), which describes distribution of the antibody in various physiological compartments;
an effector migration module, which describes T-cell infiltration within the tumor microenvironment (TME); and
the mechanism of action of the T cell redirector and dynamics within the tumor interstitial fluid of the tumor cell.

Parameters used in the model were based on in vitro activity and binding assays for the T cell redirector. Other parameters were calibrated to clinical data and to population PK parameters. The model uses virtual patients, that were defined according to sets of sampled parameters from known distributions of physiological parameters from real-world patients. Simulations of the disease course were performed using different dosing strategies that simulate a virtual clinical trial.

The exposure to the antibody was calibrated using clinical PK data and a model which predicts the movement of the antibody within different compartments of the body. The flow to and from the tumour can be edited to account for the effector to tumour cell (E:T) ratio in the tumour microenvironment (TME). Prostate tumours are “cold” tumours with relatively low levels of effector cells in the TME with a median E:T of approximately 1:20.

The model is then calibrated to consider the potency of the KLK2xCD3 bispecific antibody using in-vitro activation data and in vitro cytotoxicity data. In vitro cell binding assays identified the CD3 K_{D} as 46 nM. KLK2 receptor density was assumed to be 10,000 receptors per cell (based of experimental data from VCaPs and biopsy data). Using a combination of these factors it is possible for the model to predict the formation of tumour-bispecific-effector (TBE) trimers in the tumour microenvironment.

The next step of the model is to provide any clinical data to calibrate the maximum activity of the KLK2xCD3 bispecific antibody using PSA response data. The model then simulates virtual clinical trials on a high number of virtual patients; the simulation uses the previous calibrations regarding trimer formation, receptor occupancy and overall response rate across cohorts in order to model potential outcomes.

In this case, the QSP model guides to determining that 300 mg Q6W IV may achieve maximum TBE formation. However, evaluation of higher dose of 900 mg Q6W IV will be carried out as mechanistic PK/PD modelling predicts that PSA response may be optimized compared to 300 mg Q6W. The clinical data required to calibrate the model from the Cohort in Part 2A which is receiving 900 mg Q6W IV is still maturing.

### Example 15: RP2D

A RP2D was determined to be 3.5 mg (Day 1), 18 mg (Day 8), then 300 mg IV infusion every 6 weeks.

As of Oct. 7, 2024, 172 patients received at least one dose of the hK2xCD3 antibody. Most patients reported ≥1 related treatment-emergent adverse event (TRAE; Total, 139/172 [80.8%]; 64.3% IV; 92.2% SC). Grade ≥3 TRAEs occurred in 17/172 (9.9%) participants. One participant experienced protocol-defined dose-limiting toxicities. In the RP2D safety population (n=45), the most common TRAEs were infusion-related reactions (22.2%; Grade 1/2), fatigue (15.6%; Grade 1/2), and cytokine release syndrome (8.9%; all Grade 1). In the RP2D efficacy population (n=33), the median radiographic progression-free survival was 6.77 (95% CI 1.87, not evaluable) months.

Dosing Scheme Selected for Part 2A: Dose Expansion in Patients with Prostate Cancer with Bone or Lymph Node Metastatic Disease.

The Cohort for Part 2A is currently enrolling. There are currently 6 patients being treated with the following administration (with a plan to enroll 20 patients). On Day 1 the patient is administered a step-up dose of 3.5 mg. On Day 8 the patient is administered step-up dose of 18 mg. On Day 15 the patient is administered the treatment dose of 300 mg. The patient has a check up on Day 36 (Q3W) which is a visit only (i.e. no administration of the drug). On Day 52 the patient receives treatment dose 2 (at 300 mg). Dosing continues every six weeks with visits at three weeks. The step-up doses and treatment doses are administered by IV infusion.

Future cohorts may follow a different administration route. For example, only one step-up dose (10 mg) with a treatment dose of 300 mg. On Day 1 the patient is administered a step-up dose of 10 mg. On Day 8 the patient is administered the treatment dose of 300 mg. The patient has a check-up at Q3W which is a visit only (i.e. no administration of the drug). At Q6W the patient receives treatment dose 2 (at 300 mg). Dosing continues every six weeks with visits at three weeks.

Future cohorts may follow a different administration route. For example, to explore higher doses of up to 900 mg. On Day 1 the patient is administered a step-up dose of 10 mg. On Day 8 the patient is administered step-up dose of 300 mg. On Day 15 the patient is administered the treatment dose of 900 mg. The patient has a check up on Day 36 (Q3W) which is a visit only (i.e. no administration of the drug). On Day 52 the patient receives treatment dose 2 (at 900 mg). Dosing continues every six weeks with visits at three weeks.

Future cohorts may follow a different administration route. For example, to explore lower doses for example 150 mg. On Day 1 the patient is administered a step-up dose of 3.5 mg. On Day 8 the patient is administered step-up dose of 18 mg. On Day 15 the patient is administered the treatment dose of 150 mg. The patient has a check up on Day 36 (Q3W) which is a visit only (i.e. no administration of the drug). On Day 52 the patient receives treatment dose 2 (at 900 mg). Dosing continues every six weeks with visits at three weeks.

### Example 16: A Phase 3 Randomized, Double-Blinded, Placebo-Controlled Study of KLK2xCD3 Bispecific Antibody in Participants with Metastatic Castration-Resistant Prostate Cancer

The Study relates to a Phase 3 randomized, double-blinded, placebo-controlled Study of KLK2xCD3 bispecific antibody versus placebo for metastatic castration-resistant prostate Cancer. The KLK2xCD3 bispecific antibody is a T-cell-redirecting agent targeting KLK2 on tumor cells and CD3 on T-cells.

### Molecule Background

As of Jul. 19, 2024, 172 participants with advanced prostate cancer and >1 prior line of therapy have been treated with KLK2xCD3 bispecific antibody across multiple dose cohorts. There were no deaths related to KLK2xCD3 bispecific antibody. There was only 1 patient who met protocol-defined DLT criteria due to transient ALT and AST elevation. There were <10% related serious adverse events and only ˜1% related AEs leading to study drug discontinuation. While most patients reported at least one treatment-related adverse event (TRAE, 81%), these were mostly low grade and manageable, with only 10% of patients experiencing a Grade ≥3 TRAE. The most common TRAEs (frequency >10%) were injection site reaction (47%), fatigue (27%), and cytokine release syndrome (24%; 21% grade 1 CRS, 3% grade 2 CRS, no grade 3/4 CRS). There were 2 ICANS events. PSA50 rates have been observed from 17% to as high as 80% within dose escalation and expansion cohorts, with evidence of increased PSA50 rates at higher exposures. Overall, 38 patients remained on treatment with disease control for >6 months and 12 patients >1 year.

### Objective

To determine if the addition of the KLK2xCD3 bispecific antibody to Best Supportive Care (BSC) improves overall survival (OS) in participants with advanced mCRPC who have received all the life-prolonging therapies for which they are eligible/have access and for whom clinical trial is the best treatment option.

### Overall Design

This is a randomized, double-blinded, placebo-controlled, multinational Phase 3 treatment study to evaluate the safety and efficacy of the KLK2xCD3 bispecific antibody+BSC compared to placebo (PBO)+BSC in participants with advanced mCRPC.

Approximately 600 (tbc) participants are randomly assigned in a 2:1 ratio to either the KLK2xCD3 bispecific antibody or placebo. BSC, inclusive of as palliative external beam radiation, low dose steroids, or bone sparing agents, pain management, and any procedures/interventions to manage prostate cancer symptoms (eg nephrostomy tube or catheter placement) are given at the discretion of the physician. All participants receive background ADT, unless the participant had prior orchiectomy.

### Study Population

Histologically confirmed adenocarcinoma of the prostate as defined by PCWG3 criteria.
Disease that is metastatic to bone, lymph node, or both without evidence of metastasis to visceral organs at the time of screening
Participant must have progressed on at least 1 ARPI and is unlikely to benefit from retreatment with another ARPI.
Participants must have been previously treated with at least 2 previous taxane regimens. A taxane regimen is defined as a minimum exposure of 2 cycles of a taxane. If a patient has received only 1 taxane regimen, the patient is eligible if:
   a) Cabazitaxel is not available.
   b) The patient's physician deems the participant unsuitable to receive a second taxane regimen due to toxicity risk of prior intolerance
Participants should have been previously treated with at least 1 dose of PSMA-targeted lutetium radioligand therapy (eg, lutetium Lu-177 vipivotide tetraxetan), unless one of the following applies:
   a) PSMA-targeted lutetium radioligand therapy is unavailable, not accessible, or not clinically indicated
   b) The patient's physician deems the participant unsuitable to receive PSMA-targeted lutetium radioligand therapy.
PARPi: Should have been previously treated with a PARPi, if participant has a known germline or somatic BRCA mutation and treatment is available.
In the opinion of the investigator, the next best treatment option for the patient is a clinical trial.
Clinical laboratory parameters within the following ranges within 7 days prior to randomization. Transfusion or growth factor usage within 14 days of randomization is not allowed.
   Hemoglobin >8.0 g/dL
   Absolute neutrophil count (ANC)>1.0×109/L
   Platelet count >75×109/L
   ALT and AST≤3 xULN.
   Serum total bilirubin ≤1.5×ULN; participants with congenital bilirubinemia, such as Gilbert's syndrome, may enroll if conjugated bilirubin is >1.5×ULN.

### Treatment

The KLK2xCD3 bispecific antibody or PBO is administered IV every 6 weeks until disease progression, unacceptable toxicity, death, withdrawal of consent, or termination of the study by the Sponsor.

Participants deemed by the investigator to be receiving clinical benefit may continue study treatment beyond progression after discussion with the Sponsor. No crossover is planned.

Patients are monitored for 2 hours after infusion for the first 2 cycles only. There is no required hospitalization for post-dose monitoring, unless certain toxicities were observed after prior dose.

### Efficacy Evaluations

The primary endpoint for this study is OS.

A key secondary endpoint is rPFS as assessed by the investigator which includes computed tomography or magnetic resonance imaging scans and whole-body bone scans (Technetium-99m).

Other key secondary endpoints are PFS, including unequivocal clinical progression, and symptomatic progression.

### Study Phases:

The study consists of 3 phases: a Screening Phase, a Treatment Phase, and a Follow-up Phase.

Participants are followed until death or termination of the study. In addition to survival follow-up, data continue to be collected to evaluate secondary and other endpoints.

Participants are monitored for safety during Screening, Treatment Phases, and up to 42 days after the last dose of study medication during the follow-up phase.

### Treatment

In Cycle 1, participants receive on Day 1 step-up dose 1 (SU1) of 3.5 mg of KLK2xCD3 bispecific antibody (or placebo), on Day 8 step-up dose 2 (SU2) of 18 mg of KLK2xCD3 bispecific antibody (or placebo), and on Day 15 treatment dose of 300 mg of KLK2xCD3 bispecific antibody (or placebo). Cycle 1 ends 6 weeks following the first full treatment dose on C1D15. From Cycle 2 onwards, participants receive 300 mg of KLK2xCD3 bispecific antibody (or placebo) Q6W on Day 1. All pre-medications must be administered within 2 hours prior to study treatment.

KLK2xCD3 bispecific antibody or Placebo (PBO) is planned to be administered IV every 6 weeks until disease progression, unacceptable toxicity, death, withdrawal of consent, whichever occurs first, or termination of the study by the Sponsor. Participants deemed by the investigator to be receiving clinical benefit may continue study treatment beyond progression after discussion with the Sponsor. Patients are monitored for 2 hours after infusion for the first 2 cycles only. Participants are treated in an infusion center or other monitored setting, but post-dose monitoring in the hospital or an appropriate healthcare setting is not required throughout this study (including both step-up and all treatment doses) unless protocol-specified toxicity criteria are met for CRS, IRR, or neurologic toxicity (including ICANS). If these occur, post-dose monitoring in a hospital or observation in an appropriate healthcare is required for at least 36 hours after the next study treatment administration.

### Efficacy Evaluations

The objective of the study is to determine if the addition on KLK2xCD3 bispecific antibody to Best Supportive Care (BSC) improves overall survival (OS) in participants with advanced mCRPC who have received all the life-prolonging therapies for which they are eligible/have access and for whom clinical trial is the best treatment option.

The primary endpoint for this study is OS (Overall Survival).

A key secondary endpoint is rPFS (radiographic progression-free survival) as assessed by the investigator which includes computed tomography or magnetic resonance imaging scans and whole-body bone scans (Technetium-99m). Other key secondary endpoints are PFS (progression-free survival), including unequivocal clinical progression, and symptomatic progression.

Efficacy evaluations include radiographic disease assessments (CT scan or MRI [of chest, abdomen, and pelvis] and whole-body bone scans [99mTc]), electronic PROs, serum PSA, and assessment of symptomatic progression and survival status.

### Example 17: A Phase 3 Randomized, Open-Label Multinational Study of Metastasis-Directed Radiotherapy in Combination with a T-Cell-Redirecting Agent Targeting Human Kallikrein 2 Versus Metastasis-Directed Radiotherapy for Oligometastatic Hormone-Sensitive Prostate Cancer

The Study relates to a Phase 3 Randomized, Open-label Multinational Study of Metastasis-directed Radiotherapy in combination with a T-cell-redirecting Agent Targeting Human Kallikrein 2 Versus Metastasis-directed Radiotherapy for (Oligometastatic) Hormone-sensitive Prostate Cancer. The T-cell-redirecting Agent Targeting Human Kallikrein 2 is the KLK2xCD3 bispecific antibody of example 1.

Dosing of the KLK2xCD3 bispecific antibody is provided with 2 step-up doses (3.5 mg and 18 mg) followed by the fixed treatment dose of 300 mg Q6W by IV infusion. Please see examples 2-16 above (which describe the Phase 1 Study and the determination of the RP2D).

### Objectives

To compare radiographic progression-free survival (rPFS) of the KLK2xCD3 bispecific antibody in combination with metastasis-directed radiotherapy vs. metastasis-directed radiotherapy alone. rPFS will be assessed as time from randomization to time of first evidence of blinded independent central review (BICR)-confirmed radiographic progression of bone or soft tissue metastasis by PSMA-PET or death by any cause, whichever occurs first.
To assess the clinical benefit such as event free survival, disease free rate at 12 and 24 months, overall survival, time to initiation of castration therapy (ADT and/or ARPI), PFS2 and patient reported outcomes of the KLK2xCD3 bispecific antibody with metastasis-directed radiotherapy compared to metastasis-directed radiotherapy alone.
To compare the safety profile of the KLK2xCD3 bispecific antibody with metastasis-directed radiotherapy compared to metastasis-directed radiotherapy alone.

### Overall Design

This is a randomized, open-label multinational Phase 3 treatment study to evaluate the safety and efficacy of the KLK2xCD3 bispecific antibody+metastasis-directed radiotherapy compared to metastasis-directed radiotherapy alone in participants with mHSPC, more particularly oligometastatic HSPC.

Approximately 480 (tbc) participants are randomly assigned in a 1:1 ratio to either the KLK2xCD3 bispecific antibody+metastasis-directed radiotherapy or metastasis-directed radiotherapy alone. The study consists of 4 phases: a Screening Phase, a Treatment Phase, a Post treatment Follow-up Phase and Long-term Follow-up Phase. Efficacy, safety, pharmacokinetics (PK), and biomarkers are assessed according to the Schedule of Activities (SoA).

All participants receive metastasis directed radiotherapy starting within 21 days following randomization. In the investigational arm, the KLK2xCD3 bispecific antibody is administered for at least 12 months until rPFS by PSMA-PET, death, unacceptable toxicity or patient withdraw of consent. Patients with undetectable PSA and no rPFS by PSMA-PET (complete response) may discontinue therapy after a minimum of 12 months and enter post-treatment follow-up until radiographic progression by PSMA-PET (or death whichever comes first). Patients with complete response may resume the KLK2xCD3 bispecific antibody therapy at time of biochemical progression and without radiographic progression. Participants who have not achieved a complete response continue on therapy until rPFS by PSMA-PET. Patients may continue therapy after progression if investigator deems in the best interest of the patient.

Subsequently, all participants will go on to Long Term Follow-up Phase.

Participants are monitored for safety during Screening, and Treatment Phases and up to 30 days after the last dose of study medication during the follow-up phase. Adverse events (AEs) including clinically significant laboratory abnormalities reported as AEs, are graded and summarized using National Cancer Institute Common Terminology Criteria for Adverse Events, Version 5.

### Number of Participants

Approximately 480 (tbc) participants are randomly assigned in this study.

### Treatment Groups and Duration

Participants will be randomized in a 1:1 ratio to each treatment arms:
(1) the KLK2xCD3+metastasis-directed radiotherapy (Experimental arm)
(2) Metastasis-directed radiotherapy (Control arm)

### Study Population

Histologically confirmed adenocarcinoma of the prostate as defined by PCWG3. Primary (or documented evidence of conversion to) small cell carcinoma, carcinoid tumor, mixed NE carcinoma, large cell NE carcinoma, or sarcoma of the prostate is excluded;
No evidence metastasis to visceral organs at the time of screening scan; and
Patients treated with no more than 5 isocenters (lesions within radiation field) as determined by the treating radiation oncologist (metastasis in no more than five (distinct) locations in the patient's body);

### Additional Inclusion Criteria Include One or More of the Following Criteria:

Must have completed definitive local therapy to the primary prostate cancer with either radical prostatectomy or definitive RT to prostate (including brachytherapy), or both;
Must have at least 1 lesion visible on PSMA-PET scans;
Must have no measurable lesions by conventional imaging (Lymph nodes with short axis diameter ≤15 mm and/or equivocal signal on bone scan allowed);
Patients must be candidates for ablative metastasis-directed radiotherapy to all lesions visible on PSMA-PET and treated with no more than 5 isocenters (lesions within radiation field) as determined by the treating radiation oncologist;
PSA≥0.2 ng/mL post radical prostatectomy; or, ≥2 ng/mL from nadir PSA level for patients treated with primary radiation alone;
Must not have any known or suspected history of CNS involvement of prostate cancer.
No evidence metastasis to visceral organs at the time of screening scan;
No prior use of ARPI, immunotherapy, radioligand therapy, or chemotherapy for treatment of prostate cancer;
Have no evidence of castration resistance, defined as below:
   Have not undergone bilateral orchiectomy,
   Have not progressed while castrate (Testosterone <50 ng/dL) on ADT,
   Have non-castrate levels of testosterone (>150 ng/dL) due to testosterone supplementation prior to or at randomization;
Hematology laboratory parameters within the following ranges without transfusion or growth factors within 14 days prior to first dose of study treatment:
   Hemoglobin ≥8.0 g/dL,
   Absolute neutrophil count ≥1.0×103/μL (except for participants with benign ethnic neutropenia who may be enrolled with absolute neutrophil count less than 1.0×103/μL). Platelets ≥75×103/μL.

### Efficacy Evaluations

The primary endpoint for this study is rPFS (evaluated using PSMA-PET).

### Pharmacokinetic Evaluations

Venous blood samples are collected to measure plasma concentrations

### Safety Evaluations

Safety parameters to be analyzed are the incidence, intensity, and type of AEs and clinically significant changes in the participant's physical examination, heart rate, blood pressure, and clinical laboratory results.

### Example 18: A Phase 3 Randomized, Open-Label Study of a T-Cell-Redirecting Agent Targeting Human Kallikrein 2 (KLK2xCD3 Bispecific Antibody), in Combination with Metastasis-Directed Radiotherapy Versus Metastasis-Directed Radiotherapy Alone in Participants with Oligometastatic Hormone-Sensitive Prostate Cancer

This is a randomized, open-label Phase 3 study to evaluate the efficacy and safety of the addition of KLK2xCD3 bispecific antibody (pasritamig) to metastasis-directed radiotherapy (MDR) versus MDR alone in participants with oligometastatic hormone-sensitive prostate cancer (OMCP) based on PSMA-PET. Approximately 720 participants are randomly assigned in this study in a 1:1 ratio to receive open-label KLK2xCD3 bispecific antibody intravenous (IV) in combination with MDR (treatment arm) or MDR alone (control arm). Participants were randomly assigned in this study in a 1:1 ratio with the following stratification criteria: number of metastatic lesions on PSMA-PET (≤3 or >3), PSADT (>3 months to 9 months), and radical prostatectomy history (yes/no).

The study is summarized in Table 19.

TABLE 19

| | |
|---|---|
| Study design | Randomized, open-label, multinational, Phase 3 study. |
| Study | adult males ≥18 years with Oligometastatic Hormone- |
| population | sensitive Prostate Cancer (OMPC). |
| Total number | 720 |
| of participants | |
| Primary | To compare, radiographic progression-free survival |
| objectives | (rPFS) is assessed as time from randomization to time |
| | to first evidence of blinded independent central |
| | radiology (BICR)-confirmed radiographic progression |
| | by PSMA-PET based on RECIP 1.0 criteria, |
| | histopathologic diagnosis of prostate cancer |
| | derived from lesion on CT scan (done in conjunction |
| | with PSMA-PET), or death by any cause, whichever |
| | occurs first |
| Treatment | Open-label KLK2 × CD3 bispecific antibody |
| | administered via IV infusion. KLK2 × CD3 bispecific |
| | antibody is administered as Step-up dose 1 (SU 1) of 3.5 |
| | mg on Day 1, Step-up dose 2 (SU 2) of 18 mg on Day 8, |
| | and treatment dose (TD) of 300 mg IV Q6W afterwards. |
| | All participants are receiving metastasis-directed |
| | radiotherapy (MDR). |

### Treatment Phase

All participants in both treatment and control arm are initiating metastasis-directed radiotherapy (MDR) to all metastatic lesions visible on PSMA-PET between Day 1 (D1) to Day 14 (D314) (inclusive) of Cycle 1, and MDR must be completed by the end of Cycle 1. In participants randomized to the treatment arm, KLK2xCD3 bispecific antibody is administered by intravenous (IV) route as follows: Step-up dose 1 (SU1) of 3.5 mg on Cycle 1 Day1 (C1D1), Step-up dose 2 (SU2) of 18 mg on Cycle 1 Day8 (C1D8), and full treatment dose of 300 mg on Cycle 1 Day 15 (C1D15). Cycle 1, including step-up doses, ends after a duration of 8 weeks (i.e., 6 weeks after the first full target dose). In subsequent cycles, KLK2xCD3 bispecific antibody is administered every 6 weeks at the treatment dose of 300 mg IV. The dose regimen aligns with the RP2D regimen used in the Phase 3 study of Example 16.

Unless participants meet the primary endpoint event (rPFS on PSMA-PET by BICR) earlier, participants are receiving KLK2xCD3 bispecific antibody for a minimum of 8 cycles (approximately 12 months). Participants who have received a minimum of 8 cycles and meet the “Complete Response Criteria” at any time during treatment are required to stop therapy.

The complete response criteria (CR criteria) include:
PSA<0.2 ng/mL (RP*) or PSA<5 ng/mL (RT*) and stable (no more than 25% increase from post-MDR nadir) on 4 consecutive PSA evaluations approximately 6 weeks apart, and
Resolution of all PSMA-PET-avid lesions by BICR on 2 consecutive assessments.
(RP*: Radical prostatectomy+/−RT for local control RT*: Radiation therapy without radical prostatectomy for local control)
(RT*: Radiation therapy without radical prostatectomy for local control)

Participants on the treatment arm who do not meet the CR criteria are continuing KLK2xCD3 bispecific antibody therapy until progression on PSMA-PET by BICR.

### Post-CR Observation Phase

Participants who meet CR criteria and, therefore, stop treatment with KLK2xCD3 bispecific antibody are entering the Post-CR Observation Phase for clinical and radiographic evaluations until detection of the primary endpoint event (rPFS on PSMA-PET by BICR) or until they restart therapy.

### Re-Treatment

Participants in the post-CR observation phase may restart treatment with KLK2xCD3 bispecific antibody if following criteria are met:
Any detectable PSA if previously undetectable (RP* or RT*) or in participants with prior RT* only: PSA>25% increase from post-MDR nadir, if PSA at post-MDR nadir remained detectable (≥0.2 ng/mL) AND
A negative PSMA-PET by BICR assessed within the last 12 weeks. For participants being monitored by Q24W PSMA-PET (after 2 years on study), PSA progression per the above criteria requires an early assessment of PSMA-PET (if not done within the prior 12 weeks) to ensure rPFS event is not met prior to retreatment.

Participants who start retreatment are continuing until disease progression on PSMA-PET by BICR. No further CR criteria are applied.

### Long-Term Follow-Up Phase

In both arms of the study, participants enter a long-term follow-up phase upon detection of rPFS event on PSMA-PET by BICR. Participants are followed up for overall survival, subsequent therapies for prostate cancer and other endpoints.

### Study Population

The study population for this study consists of adult participants diagnosed with OMPC based on PSMA-PET by BICR, who are non-castrate and who have rising PSA after a definitive local therapy (radical prostatectomy, radiotherapy, or both). To select this population, the following key inclusion and exclusion criteria have been selected
Participants must have at least one M1a or M1b lesion on PMSA PET scan as determined by BICR that is amenable to MDR. If there are more than 5 distinct lesions on PSMA-PET, they must be amenable to MDR in no more than 5 treatment isocenters. There must be no definitive evidence of metastatic disease visible by conventional imaging as assessed by BICR, nor any visceral disease on PSMA-PET at time of screening by BICR or prior histological confirmation.
Participants must have rising PSA following local therapy to the prostate.
Participants must have a PSA doubling time (PSADT) of >3 and ≤18 months
Prior treatment with androgen-deprivation therapy (ADT) is allowed, however, the following criteria must be met: participants must have normal serum testosterone (>150 ng/dL) in the absence of testosterone supplementation; the participant must have no evidence of castrate-resistant disease (cannot have progressed while on active ADT in the past or have undergone bilateral orchiectomy); participants must not have immediate need for ADT.

### Endpoints

The primary endpoint is rPFS, measured as time from randomization to time of first evidence of radiographic progression on PSMA-PET imaging by BICR assessment based on RECIP 1.0, histopathologic diagnosis of prostate cancer derived from lesion on CT scan (done in conjunction with PSMA-PET), or death by any cause, whichever occurs first.

Key secondary endpoints include time to initiation of castration therapy (androgen-receptor pathway inhibitor (ARPI), androgen deprivation therapy (ADT), and/or bilateral orchiectomy), Event Free Survival, Disease-Free Rate at 36 months.

Other secondary endpoints include overall survival (OS) (for safety assessment only) and progression-free survival 2 (PFS2).

### Example 19: A Phase 3 Randomized, Double-Blinded, Placebo-Controlled Study of KLK2xCD3 Bispecific Antibody+Best Supportive Care Versus Best Supportive Care in Participants with Metastatic Castration-Resistant Prostate Cancer

### Overall Design

This is a randomized, double-blind, placebo-controlled, multinational, Phase 3 study in adult participants with mCRPC in the late-line setting. The study aims to enroll a participant population that is geographically reflective of the overall incidence/prevalence of this disease. A target of approximately 663 participants is randomly assigned in this study in a 2:1 ratio to receive KLK2xCD3 bispecific antibody (pasritamig) IV+Best standard of care (BSC) or placebo IV+BSC, respectively. All participants must be receiving background Androgen deprivation therapy (ADT) or had prior orchiectomy. All participants may receive BSC (defined as palliative external beam radiation, low dose steroids, pain medication, bone sparing agents, and needed palliative procedures). Randomization is stratified by prior PSMA-targeted radioligand therapy, prior taxane, and ECOG performance status.

The study includes a screening phase, a treatment phase, an end of treatment visit, a post-treatment follow-up phase, and if applicable, an extension phase (either long term extension or Drug Access Long-term Extension).

### Treatment Groups and Duration

In Cycle 1, participants receive on Day 1 step-up dose 1 (SU1) of 3.5 mg KLK2xCD3 bispecific antibody (or placebo), on Day 8 step-up dose 2 (SU2) of 18 mg KLK2xCD3 bispecific antibody (or placebo), and on Day 15 target dose of 300 mg KLK2xCD3 bispecific antibody (or placebo). Cycle 1 ends 6 weeks following the first full target dose on C1D15 (Cycle 1 Day 15). From Cycle 2 onwards, participants receive 300 mg KLK2xCD3 bispecific antibody (or placebo) Q6W on Day 1. All pre-medications must be administered within 2 hours prior to study treatment

The step-up and target doses of KLK2xCD3 bispecific antibody for this study represent the RP2D and were selected after review of the available nonclinical pharmacology, safety, efficacy, PK, and pharmacodynamics data from the monotherapy study of example 2-15.

As of October 2024, at the dose level of 300 mg IV (Q3W or Q6W), with step-up doses of 3.5 mg and 18 mg, there were no dose-limiting toxicities, nor any related TEAEs leading to treatment discontinuation. The rate of cytokine release syndrome (CRS) observed was 8.9% (4/45 participants), all of which were Grade 1. The safety profile at 300 mg IV dose was similar to the lower dose/exposure.

Among the participants treated at the RP2D, PSA50 response rate was 42.4%, confirmed PSA50 response was 36.4%, and the median rPFS was 6.77 months, with 39.4% participants still on treatment at time of data cutoff. The efficacy (based on confirmed PSA50 response) was close to plateau at RP2D with no further increase of efficacy at higher dose/exposure. Pharmacodynamics data also support this regimen with induction of cytokines and T-cell activation following KLK2xCD3 bispecific antibody dosing, indicative of the mechanism of action.

### Objectives

Objectives and endpoints are described in Table 20.

TABLE 20

| | |
|---|---|
| Objectives | Endpoints |

Primary

| | |
|---|---|
| To determine if KLK2 × CD3 | Overall Survival (OS) |
| bispecific antibody + BSC | |
| compared to placebo + BSC is | |
| superior in overall survival | |
| (OS). | |

Key Secondary

| | |
|---|---|
| To compare the clinical benefit | Radiographic progression-free survival |
| of KLK2 × CD3 bispecific | (rPFS) per RECIST v1.1 and PCWG3 |
| antibody + BSC to placebo + | Time to symptomatic progression |
| BSC. | Time to skeletal-related event |
| | Progression-free survival (PFS) |

Other Secondary

| | |
|---|---|
| To compare the clinical benefit | Time to PSA progression |
| of KLK2 × CD3 bispecific | Time to pain progression (TTPP), as |
| antibody + BSC to placebo + | assessed by the BPI-SF item 3 “worst |
| BSC. | pain in 24 hours” |
| | Time to deterioration in fatigue, as |
| | assessed by the EORTC QLQ-C30 FA |
| | scale |
| To compare the safety profile of | Incidence and severity of adverse |
| KLK2 × CD3 bispecific | events (AEs) |
| antibody + BSC to placebo + | Clinical laboratory test results |
| BSC. | |

### Study Population

The inclusion and exclusion criteria for enrolling participants in this study are described below.

### Inclusion Criteria

(1) must be ≥18 years of age
(2) have histologically confirmed adenocarcinoma of the prostate. Primary (or pathologic evidence of conversion to) small cell carcinoma, carcinoid tumor, mixed NE carcinoma, large cell NE carcinoma, or sarcoma of the prostate is disallowed.
(3) have mCRPC: Disease that is metastatic either to bone, any lymph node, or both without clear evidence of metastasis to visceral organs at the time of screening. Local-regional invasion (rectum, bladder) can be included.
(4) have PSA≥2 ng/mL at screening.
(5) Participants must have progressed on at least 1 ARPI and unlikely to benefit from retreatment with another ARPI.
(6) Participants should have received at least 2 previous taxane-based regimens. If a patient has received only 1 taxane regimen, the patient is eligible if Cabazitaxel is not available or the patient's physician has deemed the participant unsuitable to receive a second taxane regimen due to toxicity risk of prior intolerance
(7) Participants should have been previously treated with at least 1 dose of PSMA-targeted lutetium radioligand therapy (eg, lutetium Lu-177 vipivotide tetraxetan), unless PSMA-targeted lutetium radioligand therapy is unavailable, not accessible, or not clinically indicated.
(8) Participants should have been previously treated with PARPi, if the participant has a known germline or somatic BRCA mutation and treatment is available.
(9) The use of any other anticancer therapy or investigational agent must be discontinued for at least 2 weeks before the first dose of study treatment.
(10) Prior orchiectomy or medical castration (receiving ongoing ADT with a GnRH analog [agonist or antagonist]) prior to the first dose of study treatment and must continue this therapy throughout the treatment phase.
(11) Must be sufficiently recovered from any recent surgery or trauma.
(12) Have an ECOG performance status of 0 to 2.
(13) Have an eGFR≥30 mL/min, calculated with the CKD-epi formula (at https://www.kidney.org/professionals/gfr_calculator), before randomization. Participants with obstructive uropathy should have treatment prior to randomization (eg, foley catheter, nephrostomy tubes, etc).
(14) Participants are eligible if they have the following hepatic values:
   (a) ALT and AST≤5 xULN.
   (b) Serum total bilirubin ≤3×ULN.
(15) Participants should have the following hematologic values:
   (a) ANC≥1.0×109/L.
   (b) Hemoglobin ≥8.0 g/dL.
   (c) Platelet count ≥75×109/L

Any potential participant who meets any of the following criteria may be excluded from participating in the study:
(1) Solid organ or bone marrow transplantation.
(2) Venous thromboembolic events (eg, pulmonary embolism) within 1 month prior to the first dose of study treatment; uncomplicated (Grade ≤2) deep vein thrombosis is not exclusionary.
(3) Active autoimmune disease within the 12 months prior to signing consent that requires systemic immunosuppressive medications (eg, chronic corticosteroid, methotrexate, or tacrolimus).
(4) Active infection or condition that requires treatment with systemic antibiotics within 7 days prior to the first dose of study treatment. Prophylactic anti-infective agents are allowed.
(5) Clinically significant pulmonary compromise, particularly a requirement for supplemental oxygen use (>2 L/min by nasal cannula) to maintain adequate oxygenation.
(6) Suspected or known allergies, hypersensitivity, or intolerance to excipients of KLK2xCD3 bispecific antibody.
(7) Participant has a prior or concurrent second malignancy (other than the disease under study) for which natural history or treatment could likely interfere with any study endpoints of safety or the efficacy of the study treatment.
(8) Any of the following cardiovascular dysfunction within 6 months prior to first dose of study treatment:
   (a) Myocardial infarction
   (b) Severe or unstable angina
   (c) Clinically significant ventricular arrhythmias
   (d) Congestive heart failure (New York Heart Association class II to IV)
   (e) Transient ischemic attack
   (f) Cerebrovascular accident
(9) Has known history of either brain or leptomeningeal prostate cancer metastases.

### HIV Status

(10) Participants who are HIV-positive and meet any of the following: detectable viral load (ie, >50 copies/mL) at screening, CD4+ count<300 cells/mm3 at screening, AIDS-defining opportunistic infection within 6 months of screening, Receive treatment other than continued HAART. A change in HAART due to resistance/progression must occur at least 3 months prior to screening. A change in HAART due to toxicity is allowed up to 4 weeks prior to screening.
(11) Active or chronic HBV or HCV infection:
   (a) Seropositive for hepatitis B: defined by a positive test for HBsAg. Participants with resolved infection (ie, participants who are HBsAg negative with positive antibodies to total anti HBc) must be screened using RT PCR measurement of HBV DNA levels. Those who are RT-PCR positive are excluded.
   (b) Known hepatitis C infection or positive serologic testing for hepatitis C virus (anti-HCV) antibody. Participants with positive hepatitis C antibody due to prior resolved disease can be enrolled only if a confirmatory negative hepatitis C RNA test is obtained at screening or within 3 months prior to first dose of study treatment.
   (c) Other clinically active liver disease of infectious origin.
(12) Prior treatment with KLK2-targeted therapy.
(13) Prior treatment with any CD3-directed therapy.
(14) Received immunosuppressive doses of systemic medications, such as glucocorticoids (doses >10 mg/day prednisone or equivalent) within 3 days prior to the first dose of study treatment. A single course of glucocorticoids is permitted as prophylaxis for imaging contrast (ie, for participants with allergies to contrast). If glucocorticoids were used to treat immune-related adverse events associated with prior therapy, ≥7 days must have elapsed since the last dose of corticosteroid.
(15) Received or plans to receive any live, attenuated vaccine within 4 weeks before the first dose of study treatment. Live, attenuated influenza vaccines are permitted as late as 7 days before the study treatment.
(16) Any serious underlying medical conditions or other issue that would impair the ability of the participant to receive or tolerate the planned treatment at the study site, to understand the informed consent, or any condition for which, in the opinion of the investigator, participation would not be in the best interest of the participant (eg, compromise the well-being) or that could prevent, limit, or confound the protocol specified assessments.

### Efficacy Assessment

Efficacy evaluations include radiographic disease assessments (CT scan or MRI [of chest, abdomen, and pelvis] and whole-body bone scans [^{99m}Tc].

### Example 20: Clinical Results for the First-In-Human Phase 1 Study of KLK2xCD3 Bispecific Antibody in Participants with Advanced Prostate Cancer

### Human Pharmacokinetics

As of 19 Nov. 2024, preliminary PK results were available from participants with mCRPC in the ongoing monotherapy study described in Example 2-15. After the first IV administration of KLK2xCD3 bispecific antibody at target dose, median Tₘₐₓ occurred at the end of infusion. Mean values for Cₘₐₓ and AUC_{0-21d} (Q3W) or AUC_{0-42d} (Q6W) of KLK2xCD3 bispecific antibody increased approximately proportionally with increasing doses for the IV target dose range that has been evaluated in this study (150 mg and 300 mg Q3W, 300 mg and 900 mg Q6W [3.5 mg and 18 mg or 10 mg and 300 mg step-up doses]). Mean t_{1/2} was approximately 15.7 days.

Following multiple IV administrations of KLK2xCD3 bispecific antibody, steady state was achieved following the fifth IV administration with Q3W dosing and achieved following the third IV administration with Q6W dosing. The mean accumulation ratio (based on C_{trough}) was 1.77 for Q3W dosing and was 1.27 for Q6W dosing.

### Efficacy

As of 7 Oct. 2024, 174 participants with mCRPC have been treated (102 via SC administration and 72 via IV administration) in the monotherapy study 78278343PCR1001. An RP2D of 300 mg Q6W dose by IV infusion, with 2 step doses of 3.5 mg on Day 1 and 18 mg on Day 8, was selected and expanded. In total, 33 participants treated with target dose of 300 IV Q6W were grouped together as the RP2D-efficacy population.

Radiographic Progression-free Survival: The median rPFS was 5.88 months for participants treated across IV cohorts, and the median rPFS was 6.77 months for participants treated with IV administration at the RP2D-efficacy dose level.

Objective Response: Eighty-five of 174 (48.9%) participants across the SC and IV routes of administration had measurable disease. An objective response was observed in 7 (8.2%) participants, including 1 (1.2%) complete response and 6 (7.1%) partial response.

PSA Response: PSA decrease of 50% or more were observed at doses as low as 25 mg Q3W SC. In the RP2D-efficacy population, 42.4% achieved a PSA50 response, 36.4% of which had a confirmed PSA50 three weeks or later.

### Safety

As of 7 Oct. 2024, safety has been assessed from 174 treated participants in the ongoing monotherapy study described in Example 2-15. Forty-five participants received KLK2xCD3 bispecific antibody at target dose of 300 mg IV Q3W or Q6W and were grouped together in the RP2D-safety population.

Only 9% of participants experienced a related Grade 3 or higher AE, and no participants experienced any AEs leading to death. Across all cohorts, only 1 of 174 participants experienced either a protocol-defined DLT or a related AE leading to discontinuation of study treatment; both of these events occurred in the same participant (transient Grade 3 AST/ALT elevation) treated with SC KLK2xCD3 bispecific antibody. Notably, IV administration was much better tolerated than SC administration, with fewer related SAEs and related AEs of any grade, no dose-limiting toxicities, and no participants with a related AE leading to discontinuation of study treatment. This difference in AE profile between SC and IV administration was largely driven by a higher incidence of injection site reactions, CRS, and fatigue associated with SC administration.

At the RP2D-safety dose level (which is an IV regimen), no participants discontinued study treatment due to AEs and only 4 (8.9%) participants reported CRS, all of which were Grade 1. These CRS events did not require intervention, except for acetaminophen in 2 participants, and no recurrence of CRS was observed. No tocilizumab was administered, nor were any ICANS events observed at this dose level. The most common treatment-related AEs at the RP2D dose level were fatigue (15.6%) and infusion-related reaction (22.2%), all of which were Grade 2 or lower.

### Example 21: Translational Analyses of T-Cell Phenotypes and their Association with Clinical Efficacy in the First-In-Human (FIH) Trial of Pasritamig in Metastatic Castration-Resistant Prostate Cancer (mCRPC)

### Background

The anti-hK2/anti-CD3 bispecific antibody of the application, i.e., pasritamig, is a first-in-class bispecific T cell engager that binds CD3 and kallikrein 2 (KLK2), a protein highly and specifically expressed in prostate cancer. Pasritamig showed low rates of cytokine release syndrome and promising efficacy in a FIH trial in mCRPC (cf. examples above). Translational analyses were performed to confirm the mechanism of action and support recommended phase 2 dose (RP2D) selection.

### Methods:

Patients (pts) received pasritamig (N=178) at varying step up doses and escalating target doses of 0.5-2000 mg subcutaneously (SC) weekly (QW) or every 3 wks (Q3W) or 150-900 mg intravenously (IV) Q3W or every 6 wks (Q6W). Archival/fresh and 6-8 wk post treatment biopsies (total N=45) were analyzed for KLK2 expression, immune infiltrate and functionality. Peripheral blood mononuclear cells (PBMCs) from 34 pts were collected 24-72 h after first target dose, frequently during the first 4 cycles, then every 3 mo up to 1 y on treatment; samples were analyzed from pts with/without PSA50 response who were dosed at 800 mg SC vs 300 mg IV Q3W or Q6W. Immunophenotypes associated with activation-induced cell death (AICD) or T-cell exhaustion were analyzed by flow cytometry and machine learning.

### Results:

Most archival (30/31), fresh baseline (3/4), and post treatment (8/10) tumor biopsies showed high KLK2 expression with evidence of CD8 T cell infiltration in 5 of 7 post treatment samples. Compared with QW or Q3W cohorts, pasritamig dosed at Q6W IV maintained a reprogrammable progenitor CD8 T cell population with lower expression of the apoptotic markers activated caspase-3 and γH2AX in PBMCs (n=186), suggestive of less AICD and less terminal T cell exhaustion and correlating with better clinical efficacy. Among subsets identified by unsupervised analyses, reprogrammable progenitor T cells were positively associated with PSA50 response in the 34 pts tested, regardless of dose.

### CONCLUSIONS

Biomarker analyses of peripheral and tumor-infiltrating T cells support the proposed mechanism of action of pasritamig and recommended RP2D. Pts with a higher percentage of reprogrammable progenitor T cells were more likely to have a PSA50 response.

SEQUENCE LIST

| SEQ ID | | |
|---|---|---|
| NO | Sequence | Description |
| 1 | SYYWS | HCDR1 of hK2 binding |
| | | domain of anti-hK2/anti- |
| | | CD3 bispecific antibody |
| 2 | YIYYSGSTNYNPSLKS | HCDR2 of hK2 binding |
| | | domain of anti-hK2/anti- |
| | | CD3 bispecific antibody |
| 3 | TTIFGVVTPNFYYGMDV | HCDR3 of hK2 binding |
| | | domain of anti-hK2/anti- |
| | | CD3 bispecific antibody |
| 4 | RASQGISSYLA | LCDR1 of hK2 binding |
| | | domain of anti-hK2/anti- |
| | | CD3 bispecific antibody |
| 5 | AASTLQS | LCDR2 of hK2 binding |
| | | domain of anti-hK2/anti- |
| | | CD3 bispecific antibody |
| 6 | QQLNSYPLT | LCDR3 of hK2 binding |
| | | domain of anti-hK2/anti- |
| | | CD3 bispecific antibody |
| 7 | QVQLQESGPGLVKPSETLSLTCTVSGGSIS | VH of hK2 binding domain |
| | SYYWSWIRQPPGKGLEWIGYIYYSGSTNY | in anti-hK2/anti-CD3 |
| | NPSLKSRVTISVDTSKNQFSLKLSSVTAAD | bispecific antibody |
| | TAVYYCAGTTIFGVVTPNFYYGMDVWGQ | |
| | GTTVTVSS | |
| 8 | DIQMTQSPSFLSASVGDRVTITCRASQGISS | VL of hK2 binding domain |
| | YLAWYQQKPGKAPKFLIYAASTLQSGVPS | of anti-hK2/anti-CD3 |
| | RFSGSGSGTEFTLTISSLQPEDFATYYCQQ | bispecific antibody |
| | LNSYPLTFGGGTKVEIK | |
| 9 | QVQLQESGPGLVKPSETLSLTCTVSGGSIS | HC1 of anti-hK2/anti-CD3 |
| | SYYWSWIRQPPGKGLEWIGYIYYSGSTNY | bispecific antibody (with C- |
| | NPSLKSRVTISVDTSKNQFSLKLSSVTAAD | terminal lysine) |
| | TAVYYCAGTTIFGVVTPNFYYGMDVWGQ | |
| | GTTVTVSSASTKGPSVFPLAPSSKSTSGGT | |
| | AALGCLVKDYFPEPVTVSWNSGALTSGV | |
| | HTFPAVLQSSGLYSLSSVVTVPSSSLGTQT | |
| | YICNVNHKPSNTKVDKKVEPKSCDKTHTC | |
| | PPCPAPEAAGGPSVFLFPPKPKDTLMISRT | |
| | PEVTCVVVSVSHEDPEVKFNWYVDGVEV | |
| | HNAKTKPREEQYNSTYRVVSVLTVLHQD | |
| | WLNGKEYKCKVSNKALPAPIEKTISKAKG | |
| | QPREPQVYVLPPSREEMTKNQVSLLCLVK | |
| | GFYPSDIAVEWESNGQPENNYLTWPPVLD | |
| | SDGSFFLYSKLTVDKSRWQQGNVFSCSV | |
| | MHEALHNHYTQKSLSLSPGK | |
| 10 | DIQMTQSPSFLSASVGDRVTITCRASQGISS | LC of anti-hK2/anti-CD3 |
| | YLAWYQQKPGKAPKFLIYAASTLQSGVPS | bispecific antibody |
| | RFSGSGSGTEFTLTISSLQPEDFATYYCQQ | |
| | LNSYPLTFGGGTKVEIKRTVAAPSVFIFPPS | |
| | DEQLKSGTASVVCLLNNFYPREAKVQWK | |
| | VDNALQSGNSQESVTEQDSKDSTYSLSST | |
| | LTLSKADYEKHKVYACEVTHQGLSSPVTK | |
| | SFNRGEC | |
| 11 | RYNMN | HCDR1 of CD3 binding |
| | | domain of anti-hK2/anti- |
| | | CD3 bispecific antibody |
| 12 | SISTSSNYIYYADSVKG | HCDR2 of CD3 binding |
| | | domain of anti-hK2/anti- |
| | | CD3 bispecific antibody |
| 13 | GWGPFDY | HCDR3 of CD3 binding |
| | | domain of anti-hK2/anti- |
| | | CD3 bispecific antibody |
| 14 | RARQSIGTAIH | LCDR1 of CD3 binding |
| | | domain of anti-hK2/anti- |
| | | CD3 bispecific antibody |
| 15 | YASESIS | LCDR2 of CD3 binding |
| | | domain of anti-hK2/anti- |
| | | CD3 bispecific antibody |
| 16 | QQSGSWPYT | LCDR3 of CD3 binding |
| | | domain of anti-hK2/anti- |
| | | CD3 bispecific antibody |
| 17 | EVQLVESGGGLVKPGGSLRLSCAASGFTF | VH of CD3 binding domain |
| | SRYNMNWVRQAPGKGLEWVSSISTSSNYI | of anti-hK2/anti-CD3 |
| | YYADSVKGRFTFSRDNAKNSLDLQMSGL | bispecific antibody |
| | RAEDTAIYYCTRGWGPFDYWGQGTLVTV | |
| | SS | |
| 18 | DIQMTQSPSSLSASVGDRVTITCRARQSIG | VL of CD3 binding domain |
| | TAIHWYQQKPGKAPKLLIKYASESISGVPS | of anti-hK2/anti-CD3 |
| | RFSGSGSGTDFTLTISSLQPEDFATYYCQQ | bispecific antibody |
| | SGSWPYTFGQGTKLEIK | |
| 19 | DIQMTQSPSSLSASVGDRVTITCRARQSIG | HC2 of anti-hK2/anti-CD3 |
| | TAIHWYQQKPGKAPKLLIKYASESISGVPS | bispecific antibody (with C- |
| | RFSGSGSGTDFTLTISSLQPEDFATYYCQQ | terminal lysine) |
| | SGSWPYTFGQGTKLEIKGGSEGKSSGSGSE | |
| | SKSTGGSEVQLVESGGGLVKPGGSLRLSC | |
| | AASGFTFSRYNMNWVRQAPGKGLEWVSS | |
| | ISTSSNYIYYADSVKGRFTFSRDNAKNSLD | |
| | LQMSGLRAEDTAIYYCTRGWGPFDYWGQ | |
| | GTLVTVSSEPKSSDKTHTCPPCPAPEAAGG | |
| | PSVFLFPPKPKDTLMISRTPEVTCVVVSVS | |
| | HEDPEVKFNWYVDGVEVHNAKTKPREEQ | |
| | YNSTYRVVSVLTVLHQDWLNGKEYKCKV | |
| | SNKALPAPIEKTISKAKGQPREPQVYVYPP | |
| | SREEMTKNQVSLTCLVKGFYPSDIAVEWE | |
| | SNGQPENNYKTTPPVLDSDGSFALVSKLT | |
| | VDKSRWQQGNVFSCSVMHEALHNHYTQ | |
| | KSLSLSPGK | |
| 20 | GGSEGKSSGSGSESKSTGGS | Linker 1 |
| 21 | GGGSGGGS | Linker 2 |
| 22 | GGGSGGGSGGGS | Linker 3 |
| 23 | GGGSGGGSGGGSGGGS | Linker 4 |
| 24 | GGGSGGGSGGGSGGGSGGGS | Linker 5 |
| 25 | GGGGSGGGGSGGGGS | Linker 6 |
| 26 | GGGGSGGGGSGGGGSGGGGS | Linker 7 |
| 27 | GGGGSGGGGSGGGGSGGGGSGGGGS | Linker 8 |
| 28 | GSTSGSGKPGSGEGSTKG | Linker 9 |
| 29 | IRPRAIGGSKPRVA | Linker 10 |
| 30 | GKGGSGKGGSGKGGS | Linker 11 |
| 31 | GGKGSGGKGSGGKGS | Linker 12 |
| 32 | GGGKSGGGKSGGGKS | Linker 13 |
| 33 | GKGKSGKGKSGKGKS | Linker 14 |
| 34 | GGGKSGGKGSGKGGS | Linker 15 |
| 35 | GKPGSGKPGSGKPGS | Linker 16 |
| 36 | GKPGSGKPGSGKPGSGKPGS | Linker 17 |
| 37 | GKGKSGKGKSGKGKSGKGKS | Linker 18 |
| 38 | STAGDTHLGGEDFD | Linker 19 |
| 39 | GEGGSGEGGSGEGGS | Linker 20 |
| 40 | GGEGSGGEGSGGEGS | Linker 21 |
| 41 | GEGESGEGESGEGES | Linker 22 |
| 42 | GGGESGGEGSGEGGS | Linker 23 |
| 43 | GEGESGEGESGEGESGEGES | Linker 24 |
| 44 | GSTSGSGKPGSGEGSTKG | Linker 25 |
| 45 | PRGASKSGSASQTGSAPGS | Linker 26 |
| 46 | GTAAAGAGAAGGAAAGAAG | Linker 27 |
| 47 | GTSGSSGSGSGGSGSGGGG | Linker 28 |
| 48 | GKPGSGKPGSGKPGSGKPGS | Linker 29 |
| 49 | GSGS | Linker 30 |
| 50 | APAPAPAPAP | Linker 31 |
| 51 | APAPAPAPAPAPAPAPAPAP | Linker 32 |
| 52 | AEAAAKEAAAKEAAAAKEAAAAKEAAA | Linker 33 |
| | AKAAA | |
| 53 | GTEGKSSGSGSESKST | Linker 34 |
| 54 | ASTKGPSVFPLAPSSKSTSGGTAALGCLVK | First Ig heavy chain constant |
| | DYFPEPVTVSWNSGALTSGVHTFPAVLQS | region of anti-hK2/anti-CD3 |
| | SGLYSLSSVVTVPSSSLGTQTYICNVNHKP | bispecific antibody |
| | SNTKVDKKVEPKSCDKTHTCPPCPAPEAA | |
| | GGPSVFLFPPKPKDTLMISRTPEVTCVVVS | |
| | VSHEDPEVKFNWYVDGVEVHNAKTKPRE | |
| | EQYNSTYRVVSVLTVLHQDWLNGKEYKC | |
| | KVSNKALPAPIEKTISKAKGQPREPQVYVL | |
| | PPSREEMTKNQVSLLCLVKGFYPSDIAVE | |
| | WESNGQPENNYLTWPPVLDSDGSFFLYSK | |
| | LTVDKSRWQQGNVFSCSVMHEALHNHYT | |
| | QKSLSLSPG | |
| 55 | EPKSSDKTHTCPPCPAPEAAGGPSVFLFPP | Second Ig heavy chain |
| | KPKDTLMISRTPEVTCVVVSVSHEDPEVK | constant region of anti- |
| | FNWYVDGVEVHNAKTKPREEQYNSTYRV | hK2/anti-CD3 bispecific |
| | VSVLTVLHQDWLNGKEYKCKVSNKALPA | antibody |
| | PIEKTISKAKGQPREPQVYVYPPSREEMTK | |
| | NQVSLTCLVKGFYPSDIAVEWESNGQPEN | |
| | NYKTTPPVLDSDGSFALVSKLTVDKSRWQ | |
| | QGNVFSCSVMHEALHNHYTQKSLSLSPG | |
| 56 | RTVAAPSVFIFPPSDEQLKSGTASVVCLLN | Ig light chain constant region |
| | NFYPREAKVQWKVDNALQSGNSQESVTE | of anti-hK2/anti-CD3 |
| | QDSKDSTYSLSSTLTLSKADYEKHKVYAC | bispecific antibody |
| | EVTHQGLSSPVTKSFNRGEC | |
| 57 | QVQLQESGPGLVKPSETLSLTCTVSGGSIS | HC1 of anti-hK2/anti-CD3 |
| | SYYWSWIRQPPGKGLEWIGYIYYSGSTNY | bispecific antibody (without |
| | NPSLKSRVTISVDTSKNQFSLKLSSVTAAD | C-terminal lysine) |
| | TAVYYCAGTTIFGVVTPNFYYGMDVWGQ | |
| | GTTVTVSSASTKGPSVFPLAPSSKSTSGGT | |
| | AALGCLVKDYFPEPVTVSWNSGALTSGV | |
| | HTFPAVLQSSGLYSLSSVVTVPSSSLGTQT | |
| | YICNVNHKPSNTKVDKKVEPKSCDKTHTC | |
| | PPCPAPEAAGGPSVFLFPPKPKDTLMISRT | |
| | PEVTCVVVSVSHEDPEVKFNWYVDGVEV | |
| | HNAKTKPREEQYNSTYRVVSVLTVLHQD | |
| | WLNGKEYKCKVSNKALPAPIEKTISKAKG | |
| | QPREPQVYVLPPSREEMTKNQVSLLCLVK | |
| | GFYPSDIAVEWESNGQPENNYLTWPPVLD | |
| | SDGSFFLYSKLTVDKSRWQQGNVFSCSV | |
| | MHEALHNHYTQKSLSLSPG | |
| 58 | DIQMTQSPSSLSASVGDRVTITCRARQSIG | HC2 of anti-hK2/anti-CD3 |
| | TAIHWYQQKPGKAPKLLIKYASESISGVPS | bispecific antibody (without |
| | RFSGSGSGTDFTLTISSLQPEDFATYYCQQ | C-terminal lysine) |
| | SGSWPYTFGQGTKLEIKGGSEGKSSGSGSE | |
| | SKSTGGSEVQLVESGGGLVKPGGSLRLSC | |
| | AASGFTFSRYNMNWVRQAPGKGLEWVSS | |
| | ISTSSNYIYYADSVKGRFTFSRDNAKNSLD | |
| | LQMSGLRAEDTAIYYCTRGWGPFDYWGQ | |
| | GTLVTVSSEPKSSDKTHTCPPCPAPEAAGG | |
| | PSVFLFPPKPKDTLMISRTPEVTCVVVSVS | |
| | HEDPEVKFNWYVDGVEVHNAKTKPREEQ | |
| | YNSTYRVVSVLTVLHQDWLNGKEYKCKV | |
| | SNKALPAPIEKTISKAKGQPREPQVYVYPP | |
| | SREEMTKNQVSLTCLVKGFYPSDIAVEWE | |
| | SNGQPENNYKTTPPVLDSDGSFALVSKLT | |
| | VDKSRWQQGNVFSCSVMHEALHNHYTQ | |
| | KSLSLSPG | |
| 59 | DIQMTQSPSSLSASVGDRVTITCRARQSIG | scFv of CD3 binding domain |
| | TAIHWYQQKPGKAPKLLIKYASESISGVPS | of anti-hK2/anti-CD3 |
| | RFSGSGSGTDFTLTISSLQPEDFATYYCQQ | bispecific antibody |
| | SGSWPYTFGQGTKLEIKGGSEGKSSGSGSE | |
| | SKSTGGSEVQLVESGGGLVKPGGSLRLSC | |
| | AASGFTFSRYNMNWVRQAPGKGLEWVSS | |
| | ISTSSNYIYYADSVKGRFTFSRDNAKNSLD | |
| | LQMSGLRAEDTAIYYCTRGWGPFDYWGQ | |
| | GTLVTVSS | |
| 60 | MQSGTHWRVLGLCLLSVGVWGQDGNEE | Human CD3 epsilon |
| | MGGITQTPYKVSISGTTVILTCPQYPGSEIL | |
| | WQHNDKNIGGDEDDKNIGSDEDHLSLKEF | |
| | SELEQSGYYVCYPRGSKPEDANFYLYLRA | |
| | RVCENCMEMDVMSVATIVIVDICITGGLL | |
| | LLVYYWSKNRKAKAKPVTRGAGAGGRQ | |
| | RGQNKERPPPVPNPDYEPIRKGQRDLYSG | |
| | LNQRRI | |
| 61 | DGNEEMGGITQTPYKVSISGTTVILTCPQY | Human CD3 epsilon |
| | PGSEILWQHNDKNIGGDEDDKNIGSDEDH | extracellular domain |
| | LSLKEFSELEQSGYYVCYPRGSKPEDANF | |
| | YLYLRARVCENCMEMD | |
| 62 | MWDLVLSIALSVGCTGAVPLIQSRIVGGW | Full length hK2 |
| | ECEKHSQPWQVAVYSHGWAHCGGVLVH | |
| | PQWVLTAAHCLKKNSQVWLGRHNLFEPE | |
| | DTGQRVPVSHSFPHPLYNMSLLKHQSLRP | |
| | DEDSSHDLMLLRLSEPAKITDVVKVLGLP | |
| | TQEPALGTTCYASGWGSIEPEEFLRPRSLQ | |
| | CVSLHLLSNDMCARAYSEKVTEFMLCAG | |
| | LWTGGKDTCGGDSGGPLVCNGVLQGITS | |
| | WGPEPCALPEKPAVYTKVVHYRKWIKDTI | |
| | AANP | |
| 63 | QVQLVESGGGVVQPGRSLRLSCAASGFTF | VH of PSMA binding |
| | SSYNMNWVRQAPGKGLEWVAIIYYDESN | domain of anti-PSMA/anti- |
| | KYYADSVKGRFTISRDISKNTLYLQMNSL | CD28 bispecific antibody |
| | RAEDTAVYYCARERGRDYYGMDVWGQG | |
| | TTVTVSS | |
| 64 | SYELMQPPSVSVSPGQTARITCSGDALPKQ | VL of PSMA binding |
| | YAYWYQQKPGQAPVLVIYKDSERPSGIPV | domain of anti-PSMA/anti- |
| | RFSGSSSGTTVTLTITGVQAEDEADYYCQS | CD28 bispecific antibody |
| | ADSSGTYVFGTGTKVTVL | |

1. A method of treating prostate cancer in a human subject in need thereof, wherein the method comprises administering a therapeutically effective amount of an isolated anti-hK2/anti-CD3 bispecific antibody or a bispecific fragment thereof to the subject at a treatment dose by intravenous (IV) infusion, wherein the isolated anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18,
wherein the treatment dose is between about 150 mg and about 900 mg per administration, and the treatment dose is administered once every 1 to 6 weeks.

2. The method of claim 1, wherein the treatment dose is about 300 mg, about 150 mg, about 200 mg, about 250 mg, about 400 mg, about 500 mg, about 600 mg, about 700 mg, about 800 mg, or about 900 mg, per administration.

3. The method of claim 1, wherein the treatment dose is about 300 mg per administration, and the treatment dose is administered once every six weeks or once every three weeks.

4-7. (canceled)

8. The method of claim 1, wherein the method comprises administering to the human subject one or more step-up doses of the anti-hK2/anti-CD3 bispecific antibody or a bispecific fragment thereof, wherein the one or more step-up doses are administered prior to the treatment dose and wherein the one or more step-up doses are not higher than the treatment dose.

9-11. (canceled)

12. The method of claim 8, wherein the method comprises administering to the subject a first step-up dose and a second step-up dose of the isolated anti-hK2/anti-CD3 bispecific antibody or a bispecific fragment thereof, wherein the first step-up dose is administered prior to the second step-up dose, and the second step-up dose is administered prior to the treatment dose, and the second step-up dose is greater than the first step-up dose.

13-15. (canceled)

16. A method of treating prostate cancer in a human subject in need thereof, wherein the method comprises administering a therapeutically effective amount of an anti-hK2/anti-CD3 bispecific antibody or a bispecific fragment thereof to the human subject at a treatment dose by intravenous (IV) infusion, wherein the anti-hK2/anti-CD3 bispecific antibody or bispecific fragment thereof comprises a first antigen binding domain that binds specifically to hK2, and a second antigen binding domain that binds specifically to CD3, wherein:
a. the first domain that binds hK2 comprises a first heavy chain variable region (VH1) of SEQ ID NO: 7 and a first light chain variable region (VL1) of SEQ ID NO: 8, and
b. the second domain that binds CD3 comprises a second heavy chain variable region (VH2) of SEQ ID NO: 17 and a second light chain variable region (VL2) of SEQ ID NO: 18,
wherein the method comprises administering to the subject a first step-up dose and a second step-up dose of the anti-hK2/anti-CD3 bispecific antibody or a bispecific fragment thereof, wherein the first step-up dose is administered prior to the second step-up dose, and wherein the second step-up dose is administered prior to the treatment dose, wherein the first step-up dose and the second step-up dose are administered via intravenous infusion, and wherein:
(a) the first step-up dose is about 3.5 mg;
(b) the second step-up dose is about 18 mg; and
(c) the treatment dose is about 300 mg.

17. The method of claim 16, wherein the treatment dose is administered once every six weeks.

18. (canceled)

19. The method of claim 1, wherein the first domain that binds hK2 comprises a Fab and the second domain that binds CD3 comprises a scFv.

20-21. (canceled)

22. The method of claim 19, wherein the first domain that binds hK2 is conjugated to a first immunoglobulin (Ig) heavy chain constant region, or a fragment of the first Ig heavy chain constant region, and the second domain that binds CD3ε is conjugated to a second immunoglobulin (Ig) heavy chain constant region, or a fragment of the second Ig heavy chain constant region, and the antibody or the bispecific fragment thereof comprises the following amino acid mutations:
(a) L234A_L235A_D265S_T350V_T366L_K392L_T394W in the first Ig heavy chain constant region, or the fragment of the first Ig heavy chain constant region, and
(b) L234A_L235A_D265S_T350V_L351Y_F405A_Y407V in the second Ig heavy chain constant region, or the fragment of the second Ig heavy chain constant region,
wherein numbering of the amino acid mutations is of the EU index.

23-27. (canceled)

28. The method of claim 22, wherein the anti-hK2/anti-CD3 antibody or a bispecific fragment thereof, comprises (1) a first heavy chain (HC1) which is at least 95% identical to the amino acid sequence of SEQ ID NO: 57, a first light chain (LC1) that is at least 95% identical to the amino acid sequence of SEQ ID NO: 10 and a second heavy chain (HC2) which is at least 95% identical to the HC2 of SEQ ID NO: 58; or (2) a HC1 which at least 95% identical to the HC1 of SEQ ID NO: 9, a LC1 that is at least 95% identical to the LC1 of SEQ ID NO: 10 and a HC2 which is at least 95% identical to the HC2 of SEQ ID NO: 19.

29. A method of treating prostate cancer in a human subject in need thereof, wherein the method comprises intravenously administering to the human subject a treatment dose of 300 mg per administration of an anti-hK2/anti-CD3 bispecific antibody, once every three weeks or once every six weeks, wherein the anti-hK2/anti-CD3 antibody comprises (1) a first heavy chain (HC1) of SEQ ID NO: 57, a first light chain (LC1) of SEQ ID NO: 10 and a second heavy chain (HC2) of SEQ ID NO: 58; or (2) the HC1 of SEQ ID NO: 9, the LC1 of SEQ ID NO: 10 and the HC2 of SEQ ID NO: 19.

30-31. (canceled)

32. The method of claim 1, wherein
(1) the prostate cancer is prostate adenocarcinoma that has small cell or neuroendocrine (NE) features, optionally the prostate adenocarcinoma that does not comprise small cell carcinoma, carcinoid tumor, mixed NE carcinoma, or large cell NE carcinoma;
(2) the prostate cancer is a metastatic castration resistant prostate cancer (mCRPC), optionally the mCRPC that is metastatic to bone and/or lymph node, without evidence of metastasis to visceral organs, optionally the subject has no known prior history of CNS involvement in prostate cancer, optionally the subject has a known germline or somatic BRCA mutation;
(3) the prostate cancer is a metastatic prostate cancer, or the prostate cancer is metastatic to visceral tissue, optionally, the subject has received prior treatment with at least one androgen receptor (AR)-targeted therapy, optionally the subject has received no more than two prior taxane therapies;
(4) the subject has an oligometastatic metastatic castration-sensitive prostate cancer (mCSPC), optionally the subject has recurrent disease after definitive treatment to prostate, wherein the prostate cancer is metastatic to bone and/or lymph node, without evidence of metastasis to visceral organs, optionally the subject has no more than five total lesions on imaging;
(5) the subject has non-castrate levels of testosterone of greater than 150 ng/mL: or
(6) the prostate cancer is a metastatic hormone sensitive prostate cancer (mHSPC), optionally the mHSPC is a mHSPC with metastasis in no more than five locations in the subject's body, a mHSPC with metastasis in no more than five locations in the subject's body but with no evidence of metastasis to visceral organs, or a mHSPC with non-castrate levels of testosterone, and optionally, the subject has not undergone orchiectomy.

33. (canceled)

34. The method of claim 32, wherein
(1) the subject has received prior treatment for metastatic castration resistant prostate cancer (mCRPC) with at least one androgen receptor (AR)-targeted therapy, or at least one chemotherapy;
(2) the subject has received prior treatment for mCRPC with at least one androgen receptor (AR)-targeted therapy, wherein the prostate cancer is mCRPC that is metastatic to bone and/or lymph node, without evidence of metastasis to visceral organs, optionally the subject has no known prior history of CNS involvement in prostate cancer;
(3) the subject has received no more than two prior taxane therapies, optionally, the subject has also received prior treatment for mCRPC with at least one androgen receptor (AR)-targeted therapy, wherein the prostate cancer is mCRPC that is metastatic to bone and/or lymph node, without evidence of metastasis to visceral organs, optionally the subject has no known prior history of CNS involvement in prostate cancer;
(4) the subject has received prior treatment for mCRPC with at least one taxane regimen, optionally, the subject has also received prior treatment for mCRPC with at least one androgen receptor (AR)-targeted therapy, wherein the prostate cancer is mCRPC that is metastatic to bone and/or lymph node, without evidence of metastasis to visceral organs, optionally the subject has no known prior history of CNS involvement in prostate cancer;
(5) the subject has received prior treatment for mCRPC with two taxane regimens, optionally, the subject has also received prior treatment for mCRPC with at least one androgen receptor (AR)-targeted therapy, wherein the prostate cancer is mCRPC that is metastatic to bone and/or lymph node, without evidence of metastasis to visceral organs, optionally the subject has no known prior history of CNS involvement in prostate cancer;
(6) the subject has received prior treatment for mCRPC with at least one dose of PSMA-targeted lutetium radioligand therapy, optionally, the subject has also received prior treatment for mCRPC with at least one androgen receptor (AR)-targeted therapy, wherein the prostate cancer is mCRPC that is metastatic to bone and/or lymph node, without evidence of metastasis to visceral organs, optionally the subject has no known prior history of CNS involvement in prostate cancer;
(7) the subject has received prior treatment for mCRPC with a polyadenosine diphosphate-ribose polymerase inhibitors (PARPi), optionally, the subject has also received prior treatment for mCRPC with at least one androgen receptor (AR)-targeted therapy, wherein the prostate cancer is mCRPC that is metastatic to bone and/or lymph node, without evidence of metastasis to visceral organs, optionally the subject has no known prior history of CNS involvement in prostate cancer;
(8) the subject has received (a) prior treatment for mCRPC with at least one androgen receptor (AR)-targeted therapy, and (b) prior treatment for mCRPC with at least one taxane regimen, wherein the prostate cancer is mCRPC that is metastatic to bone and/or lymph node, without evidence of metastasis to visceral organs;
(9) the subject has received (a) prior treatment for mCRPC with at least one androgen receptor (AR)-targeted therapy, (b) prior treatment for mCRPC with at least one taxane regimen, and (c) prior treatment for mCRPC with at least one PARPi, wherein the prostate cancer is mCRPC that is metastatic to bone and/or lymph node, without evidence of metastasis to visceral organs;
optionally, the subject in (7)-(9) has a known germline or somatic BRCA mutation;
optionally, the androgen receptor (AR)-targeted therapy is selected from the group consisting of abiraterone acetate, apalutamide, enzalutamide, and darolutamide; and the PSMA-targeted lutetium radioligand therapy is lutetium Lu-177 vipivotide tetraxetan.

35-45. (canceled)

46. The method of claim 34, wherein the serum Prostate-Specific Antigen (PSA) level of the subject measured once the prior treatment with the androgen receptor (AR)-targeted therapy, the taxane therapy, the PSMA-targeted lutetium radioligand therapy or the PARPi has been completed, is not decreased compared to the PSA level measured before the administration of the androgen receptor (AR)-targeted therapy, the taxane therapy, the PSMA-targeted lutetium radioligand therapy or the PARPi, respectively.

47-53. (canceled)

54. The method of claim 1,
wherein the method provides one or more of:
(1) a decrease in the serum Prostate-Specific Antigen (PSA) level of the subject, wherein the decrease is relative to the PSA level of the subject prior to the administration of the anti-hK2/anti-CD3 bispecific antibody or a bispecific fragment thereof, optionally, the PSA level decreases by 50% or more compared to the PSA level of the subject prior to the administration of the anti-hK2/anti-CD3 bispecific antibody or a bispecific fragment thereof;
(2) no disease progression in the subject, wherein disease progression is assessed according to the Prostate Cancer Working Group 3 (PCWG3) Criteria (Scher et al. 2016 Journal of Clinical Oncology 34(12): 1402-1418); and
(3) a Partial Response or better according to RECIST version 1.1 response criteria (Eisenhauer et al. 2009 European Journal of Cancer 45: 228-247) without evidence of bone progression according to PCWG3 in the subject; and/or
wherein:
(1) no Adverse Event of Grade 3 or higher (according to the National Cancer Institute Common Terminology Criteria for Adverse Events (NCI-CTCAE) version 5.0) is observed in the subject,
(2) Adverse Events of Grade 3 or higher (according to the National Cancer Institute Common Terminology Criteria for Adverse Events (NCI-CTCAE) version 5.0) are observed in 20% or fewer of subjects in a cohort of at least 20 subjects;
(3) no event of Injection Site Reaction (ISR) of Grade 3 or higher (according to the National Cancer Institute Common Terminology Criteria for Adverse Events (NCI-CTCAE) version 5.0) is observed in the subject;
(4) events of Injection Site Reactions (ISR) (according to the National Cancer Institute Common Terminology Criteria for Adverse Events (NCI-CTCAE) version 5.0) are observed in 10% or fewer subjects in a cohort of at least 20 subjects,
(5) no event of Cytokine Release Syndrome (CRS) of Grade 3 or higher (according to the American Society for Transplantation and Cellular Therapy (ASTCT) guidelines) is observed in the subject;
(6) events of Cytokine Release Syndrome (CRS) of Grade 3 or higher (according to the American Society for Transplantation and Cellular Therapy (ASTCT) guidelines) are observed in 15% or fewer of subjects in a cohort of at least 20 subjects;
(7) events of Cytokine Release Syndrome (CRS) of Grade 2 or higher (according to the American Society for Transplantation and Cellular Therapy (ASTCT) guidelines) are observed in 15% or fewer of subjects in a cohort of at least 20 subjects; and/or
(8) the method does not induce a Dose Limiting Toxicity (DLT) in the subject.

55-65. (canceled)

66. The method of claim 1, wherein the anti-hK2/anti-CD3 bispecific antibody or a bispecific fragment thereof, is administered as a monotherapy.

67. The method of claim 1, wherein the method comprises administering to the subject one or more additional therapeutic agent, optionally the one or more additional therapeutic agent is one or more additional anti-cancer therapies, or one or more immune modulating agents.

68-69. (canceled)

70. The method of claim 1, wherein the anti-hK2/anti-CD3 bispecific antibody or a bispecific fragment thereof, is not administered to the subject in combination with a combination dose of a PSMAxCD28 bispecific antibody, wherein the anti-PSMA/anti-CD28 bispecific antibody comprises a binding domain that binds specifically to PSMA, and a binding domain that binds specifically to CD28, wherein the PSMA binding domain comprises a HCDR1, a HCDR2 and a HCDR3 of a heavy chain variable region of SEQ ID NO: 63, and a LCDR1, a LCDR2, and a LCDR3 of a light chain variable region of SEQ ID NO: 64, and the combination dose of the anti-PSMA/anti-CD28 bispecific antibody is 0.5-2000 mg per administration.

71-77. (canceled)

78. The method of claim 32, wherein the prostate cancer is mHSPC, and the method further comprises administering metastasis-directed radiotherapy (MDR) to the subject.

79. The method of claim 1, wherein the anti-hK2/anti-CD3 bispecific antibody is pasritamig.

80. (canceled)

81. The method of claim 29, wherein the subject has a metastatic castration-resistant prostate cancer (mCRPC), and wherein the subject has received (a) prior treatment with at least one androgen receptor (AR) targeted therapy, and (b) prior treatment with at least one taxane regimen.

82. The method of claim 29, wherein the subject has a metastatic castration-resistant prostate cancer (mCRPC) and a germline or somatic BRCA mutation, and wherein the subject has received (a) prior treatment with at least one androgen receptor (AR) targeted therapy, (b) prior treatment with at least one taxane regimen, and (c) prior treatment with at least one PARPi.
